SEQUENCE LISTING (419 sequences)

SEQ ID NO: 1 - DNA, 351 bp, Homo sapiens
caggtgcagctgcaggagtcgggcccaggactggtgaagccttcacagaccctgtccctcacctgcactgtctctggtggctccatcagccgtggtaattactactggagctggatccgccagcacccagggaagggcctggagtggattgggtacatcttttacagtgggagcacctactacaacccgtccctcaagagtcgagttaccatatcagtagacacgtctaagaaccagttctccctgaaactgagctctgtgactgccgcggacacggccgtatattactgtgcgagagatggctacggtatggacgtctggggccaagggaccacggtcaccgtctcctca

SEQ ID NO: 2 - protein, 117 aa, Homo sapiens
QVQLQESGPGLVKPSQTLSLTCTVSGGSISRGNYYWSWIRQHPGKGLEWIGYIFYSGSTYYNPSLKSRVTISVDTSKNQFSLKLSSVTAADTAVYYCARDGYGMDVWGQGTTVTVSS

SEQ ID NO: 3 - DNA, 324 bp, Homo sapiens
gaaattgtgttgacgcagtctccaggcaccctgtctttgtctccaggggaaagagccaccctctcctgcagggccagtcagagtgttagcagcagctacttagtttggtaccagcagaaacctggccaggctcccaggctcctcctctatgatgcatccagcagggccactggcatcccagacaggttcagtggcagtgggtctgggacagacttcactctcaccatcagcagactggagcctgaagattttgcagtgtattactgtcagcagtatggtaactcaccgctcactttcggcggagggaccaaggtagagatcaaa

SEQ ID NO: 4 - protein, 108 aa, Homo sapiens
EIVLTQSPGTLSLSPGERATLSCRASQSVSSSYLVWYQQKPGQAPRLLLYDASSRATGIPDRFSGSGSGTDFTLTISRLEPEDFAVYYCQQYGNSPLTFGGGTKVEIK

SEQ ID NO: 5 - DNA, 348 bp, Homo sapiens
gaggtgcagctggtggagtctgggggaggcctggtcaagcctggggggtccctgagactctcctgtgcagcctctggattcaccctcagtagctatagcatgaactgggtccgccaggctccagggaaggggctggagtgggtctcatccattagtagtagtagtagttacatacactatgcagactcagtgaagggccgattcaccatctccagagacaacgccaagaactcactgtatctgcaaatgaacagcctgagagccgaggacacggctgtgtattactgtgcgagagctccggtggtttttgactactggggccagggaaccctggtcaccgtctcctca

SEQ ID NO: 6 - protein, 116 aa, Homo sapiens
EVQLVESGGGLVKPGGSLRLSCAASGFTLSSYSMNWVRQAPGKGLEWVSSISSSSSYIHYADSVKGRFTISRDNAKNSLYLQMNSLRAEDTAVYYCARAPVVFDYWGQGTLVTVSS

SEQ ID NO: 7 - DNA, 327 bp, Homo sapiens
gaaattgtgttgacgcagtctccaggcaccctgtctttgtctccaggggaaagagccaccctctcctgcagggccagtcagagtgttagcagcagctacttagcctggtaccagcagaagcctggccaggctcccaggctcctcatctctggtgcatccagcagggccactggcatcccagacaggttcagtggcagtgggtctgggacagacttcactctcaccatcagcagactggagcctgaagattttgcagtgtattactgtcagcagtatggtggctcactcacgtggacgttcggccaagggaccaaggtggaaatcaaa

SEQ ID NO: 8 - protein, 109 aa, Homo sapiens
EIVLTQSPGTLSLSPGERATLSCRASQSVSSSYLAWYQQKPGQAPRLLISGASSRATGIPDRFSGSGSGTDFTLTISRLEPEDFAVYYCQQYGGSLTWTFGQGTKVEIK

SEQ ID NO: 9 - DNA, 351 bp, Homo sapiens
caggtgcagctgcaggagtcgggcccaggactggtgaagccttcacagaccctgtccctcacctgcactgtctctggtggctccatcaacagtggtggttactactggagctggatccgccagcacccagggaagggcctggagtggattgggtacatcttttacagtgggagcacctactacaacccgtccctcaagagtcgagttaacatatcagtagacacgtctaagaaccagttctccctgaagatgagctctgtgactgccgcggacacggccgtatattactgtgcgagagatcaggatggatttgactactggggccagggaaccctggtcaccgtctcctca

SEQ ID NO: 10 - protein, 117 aa, Homo sapiens
QVQLQESGPGLVKPSQTLSLTCTVSGGSINSGGYYWSWIRQHPGKGLEWIGYIFYSGSTYYNPSLKSRVNISVDTSKNQFSLKMSSVTAADTAVYYCARDQDGFDYWGQGTLVTVSS

SEQ ID NO: 11 - DNA, 339 bp, Homo sapiens
gatattgtgatgacccagactccactctccctgcccgtcacccctggagagccggcctccatctcctgcaggtctagtcagagcctcttggatagtgatgatggaaacacctatttggactggtacctgcagaagccagggcagtctccacagctcctgatctatacgctttcctatcgggcctctggagtcccagacaggttcagtggcagtgggtcaggcactgatttcacactgaaaatcagcagggtggaggctgaggatgttggagtttattactgcatgcaacgtatagagtttccttggacgttcggccaagggaccaaggtggaaatcaaa

SEQ ID NO: 12 - protein, 113 aa, Homo sapiens
DIVMTQTPLSLPVTPGEPASISCRSSQSLLDSDDGNTYLDWYLQKPGQSPQLLIYTLSYRASGVPDRFSGSGSGTDFTLKISRVEAEDVGVYYCMQRIEFPWTFGQGTKVEIK

SEQ ID NO: 13 - DNA, 348 bp, Homo sapiens
caggtgcagctggtgcagtctggggctgaggtgaagaagcctggggcctcagtgaaggtctcctgtaaggcttctggatacaccttcaccagttatgatatcaactgggtgcgacaggccactggacaagggcttgagtggatgggatggatgaaccctaacagtggtaacacaggctatgcacagaagttccagggcagaatcaccatgaccaggaacacctccataagcacagcctacatggagctgagcagcctgagatctgaggacacggccgtgttttactgtgcgagaggcgactggtacgttgactactggggccagggaaccctggtcaccgtctcctca

SEQ ID NO: 14 - protein, 116 aa, Homo sapiens
QVQLVQSGAEVKKPGASVKVSCKASGYTFTSYDINWVRQATGQGLEWMGWMNPNSGNTGYAQKFQGRITMTRNTSISTAYMELSSLRSEDTAVFYCARGDWYVDYWGQGTLVTVSS

SEQ ID NO: 15 - DNA, 339 bp, Homo sapiens
gacatcgtgatgacccagtctccagactccctggctgtgtctctgggcgagagggccaccatcagctgcaagtccagccagagtgttttatacagctccaacaataagaactacttagcttggtaccagcagaaaccaggacagcctcctaacctgctcatttactgggcatctacccgggaatccggggtccctgaccgattcagtggcagcgggtctgggacagatttcactctcaccatcagcagcctgcaggctgaagatgtggcagtttattactgtcagcaatattatagtattcctcgcagttttggccaggggaccaaactggagatcaaa

SEQ ID NO: 16 - protein, 113 aa, Homo sapiens
DIVMTQSPDSLAVSLGERATISCKSSQSVLYSSNNKNYLAWYQQKPGQPPNLLIYWASTRESGVPDRFSGSGSGTDFTLTISSLQAEDVAVYYCQQYYSIPRSFGQGTKLEIK

SEQ ID NO: 17 - DNA, 360 bp, Homo sapiens
caggtgcaggtgcaggagtcgggcccaggactggtgaagccttcggagaccctgtccctcacctgcactgtctctggtggctccatcagtagtcattactggagctggatccggcagcccgccgggaagggactggagtggattgggcgtatctataccagtgggagcaccaactacaacccctccctcaagagtcgagtcaccatgtcagtagacacgtccaagaagcagttctccctgaagctgagctctgtgaccgccgcggacacggccgtgtattactgtgcgagagaaggggattactactaccactacggtatggacgtctggggccaagggaccacggtcaccgtctcctca

SEQ ID NO: 18 - protein, 120 aa, Homo sapiens
QVQVQESGPGLVKPSETLSLTCTVSGGSISSHYWSWIRQPAGKGLEWIGRIYTSGSTNYNPSLKSRVTMSVDTSKKQFSLKLSSVTAADTAVYYCAREGDYYYHYGMDVWGQGTTVTVSS

SEQ ID NO: 19 - DNA, 336 bp, Homo sapiens
gatattgtgatgactcagtctccactctccctgcccgtcacccctggagagccggcctccatctcctgcaggtctagtcagagcctcctgcatagtattggatacaactatttggattggttcctgcagaagccggggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactccgatcaccttcggccaagggacacgactggagattaaa

SEQ ID NO: 20 - protein, 112 aa, Homo sapiens
DIVMTQSPLSLPVTPGEPASISCRSSQSLLHSIGYNYLDWFLQKPGQSPQLLIYLGSNRASGVPDRFSGSGSGTDFTLKISRVEAEDVGVYYCMQALQTPITFGQGTRLEIK

SEQ ID NO: 21 - DNA, 374 bp, Homo sapiens
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgaagactccgtgaagggccgattcaccatctccagagacaattccaagaacacgctgtatctgcaaatgaacagcctgagagccgaggacacggctgtgtattactgtgcgagaagggcgtataactggaactacgaggactactactacagtatggacgtctggggccaagggaccacggtcaccgtctcctc

SEQ ID NO: 22 - protein, 124 aa, Homo sapiens
QVQLVESGGGVVQPGRSLRLSCAASGFTFSSYGMHWVRQAPGKGLEWVAVIWYDGSNKYYEDSVKGRFTISRDNSKNTLYLQMNSLRAEDTAVYYCARRAYNWNYEDYYYSMDVWGQGTTVTVS

SEQ ID NO: 23 - DNA, 321 bp, Homo sapiens
gacatccagatgacccagtctccatcctccctgtctgcatctgtaggaaacagagtcaccatcacttgccgggcaagtcagagcattagcaactatttaaattggtatcagcagaaaccagggaaagcccctaagctcctgatctatgctacatccaatttgcaaagtggggtcccatcaaggttcagtggccgtggatctgggacagatttcactctcaccatcagcagtctgcaacctgaagattttgcaacttactactgtcaacagagttacagtaccccattcactttcggccctgggaccaaagtggatatcaaa

SEQ ID NO: 24 - protein, 107 aa, Homo sapiens
DIQMTQSPSSLSASVGNRVTITCRASQSISNYLNWYQQKPGKAPKLLIYATSNLQSGVPSRFSGRGSGTDFTLTISSLQPEDFATYYCQQSYSTPFTFGPGTKVDIK

SEQ ID NO: 25 - DNA, 363 bp, Homo sapiens
caggtgcagctgcaggagtcgggcccaggactggtgaagccttcacagaccctgtccctcacctgcactgtctctggtggctccatcagcagtggtggttactactggagctggatccgccagcacccagggaagggcctggagtggattgggtacatctataacagtgggagcacctattacaacccgtccctcaagagtcgaggcaccatatcagcagacacgtctaagaaccagttctccctgaagctgagctctgtgactgccgcggacacggccgtgtattactgtgcgagagggagcagctcgtactactggtacttcgatctctggggccgtggcaccctggtcactgtctcctca

SEQ ID NO: 26 - protein, 121 aa, Homo sapiens
QVQLQESGPGLVKPSQTLSLTCTVSGGSISSGGYYWSWIRQHPGKGLEWIGYIYNSGSTYYNPSLKSRGTISADTSKNQFSLKLSSVTAADTAVYYCARGSSSYYWYFDLWGRGTLVTVSS

SEQ ID NO: 27 - DNA, 321 bp, Homo sapiens
gacatccagatgacccagtctccatcctccatgtctgcatctgtaggagacagagtcaccatcacttgccgggcaagtcagagcattagcagtaatttaaattggtatcagcagaaaccagggaaagcccctaagctcctgatctatggtgcatccagtttgcaaagtggggtcccatcaaggttcagtggcagtggatctgggacagatttcactctcaccatcagcagtctgcaacctgaagattttgcaacttactactgtcaacagagttacagtaccccatatactttcggccctgggaccaaagtggatatcaaa

SEQ ID NO: 28 - protein, 107 aa, Homo sapiens
DIQMTQSPSSMSASVGDRVTITCRASQSISSNLNWYQQKPGKAPKLLIYGASSLQSGVPSRFSGSGSGTDFTLTISSLQPEDFATYYCQQSYSTPYTFGPGTKVDIK

SEQ ID NO: 29 - DNA, 351 bp, Homo sapiens
caggtgcaactgcaggagtcgggcccaggactggtgaagccttcggagaccctgtccctcacctgcactgtctctggtgactccatcagtagttactactggagctggatccggcagcccccagggaagggactggagtggattgggtatgtctattacagtgggagcaccaactacaacccctccctcaagagtcgagtcaccatgtcagtagacacgtccaagaaccagttctccctgaagctgagctctgtgaccgctgcggacacggccgtgtattactgtgcgagagatctggaactctatgcttttgatatctggggccaagggacaatggtcaccgtctcttca

SEQ ID NO: 30 - protein, 117 aa, Homo sapiens
QVQLQESGPGLVKPSETLSLTCTVSGDSISSYYWSWIRQPPGKGLEWIGYVYYSGSTNYNPSLKSRVTMSVDTSKNQFSLKLSSVTAADTAVYYCARDLELYAFDIWGQGTMVTVSS

SEQ ID NO: 31 - DNA, 324 bp, Homo sapiens
gaaattgtgttgacgcagtttccaggcaccctgtctttgtctccaggggaaagagtcaccctctcctgcagggccagtcagagtgttagcagtacctacgtagtctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagcatggaagctcaccgtggacgttcggccaagggaccaaggtggaaatcaaa

SEQ ID NO: 32 - protein, 108 aa, Homo sapiens
EIVLTQFPGTLSLSPGERVTLSCRASQSVSSTYVVWYQQKPGQAPRLLIYGASSRATGIPDRFSGSGSGTDFTLTISRLEPEDFAVYYCQQHGSSPWTFGQGTKVEIK

SEQ ID NO: 33 - DNA, 363 bp, Homo sapiens
caggtgcagctgcaggagtcgggcccaggactggtgaagccttcacagaccctgtccctcacctgcactgtctctggtggctccatcagcagtggtggttactactggagctggatccgccagcacccagggaagggcctggagtggattgggtacatctataacagtgggagcacctattacaacccgtccctcaagagtcgaggcaccatatcagcagacacgtctaagaaccagttctccctgaagctgagctctgtgactgccgcggacacggccgtgtattactgtgcgagagggagcagttcgtactactggtacttcgatctctggggccgtggcaccctggtcactgtctcctca

SEQ ID NO: 34 - protein, 121 aa, Homo sapiens; identical to SEQ ID NO: 26

SEQ ID NO: 35 - DNA, 321 bp, Homo sapiens
gacatccagatgacccagtctccatcctccatgtctgcatctgtaggagacagagtcaccatcacttgccgggcaagtcagcgcattagcagtaatttaaattggtatcagcagaaaccagggaaagcccctaagttcctgatctatggtgcatccagtttgcaaagtggggtcccatcaaggttcagtggcagtggatctgggacagatttcactctcaccatcagcagtctgcgacctgaagattttgcaacttactactgtcaacagagttacagtaccccatatactttcggccctgggaccaaagtggatatcaaa

SEQ ID NO: 36 - protein, 107 aa, Homo sapiens
DIQMTQSPSSMSASVGDRVTITCRASQRISSNLNWYQQKPGKAPKFLIYGASSLQSGVPSRFSGSGSGTDFTLTISSLRPEDFATYYCQQSYSTPYTFGPGTKVDIK

SEQ ID NO: 37 - DNA, 375 bp, Homo sapiens
caggtacagctgcagcagtcgggtccaggactggtgaagccctcgcagaccctctcactcacctgtgccatctccggggacagtgtctctagcaacagtgctgcttggaactggatcaggcagtccccatcgagaggccttgagtggctgggaaggacatactacatgtccaagtggtataatgattatgcagtatctgtgaaaagtcgaataaccatcaacccagacacatccaagaaccagttctccctgcagctgaactctgtgactcccgaggacacggctgtgtattactgtgcaagagcgcgatattgtggtggtgactgctatgactttgactactggggccagggaaccctggtcaccgtctcctca

SEQ ID NO: 38 - protein, 125 aa, Homo sapiens
QVQLQQSGPGLVKPSQTLSLTCAISGDSVSSNSAAWNWIRQSPSRGLEWLGRTYYMSKWYNDYAVSVKSRITINPDTSKNQFSLQLNSVTPEDTAVYYCARARYCGGDCYDFDYWGQGTLVTVSS

SEQ ID NO: 39 - DNA, 321 bp, Homo sapiens
gacatccagatgacccattctccatcctccctgtcggcatctgtaggagacagagtcaccatcacttgccgggcaagtcagagcattagcatctctttaaactggtatcagcagaagccagggaacgcccctaagctcctgatctatgctgcatccagtttgcaaagtgggatcccatcaaggttcagtggcagtgggtctgggacagatttcactctcaccatcagcagtctgcaacctgaagattttgcaacttactactgtcaacagagtttcagtaccccgtggacgttcggccaagggaccaaggtggaaatcaaa

SEQ ID NO: 40 - protein, 107 aa, Homo sapiens
DIQMTHSPSSLSASVGDRVTITCRASQSISISLNWYQQKPGNAPKLLIYAASSLQSGIPSRFSGSGSGTDFTLTISSLQPEDFATYYCQQSFSTPWTFGQGTKVEIK

SEQ ID NO: 41 - DNA, 352 bp, Homo sapiens
caggtgcagctgcaggagtcgggcccaggactggtgaagccttcggagaccctgtccctcacctgcactgtctctggtggctccatcagtagtttctactggagctggatccggcagcccccagggaagggactggaatggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcgagagatctggaactctatgcttttgatgtctggggccaggggacaatggtcaccgtctcttcag

SEQ ID NO: 42 - protein, 117 aa, Homo sapiens
QVQLQESGPGLVKPSETLSLTCTVSGGSISSFYWSWIRQPPGKGLEWIGYIYYSGSTNYNPSLKSRVTISVDTSKNQFSLKLSSVTAADTAVYYCARDLELYAFDVWGQGTMVTVSS

SEQ ID NO: 43 - DNA, 324 bp, Homo sapiens
gaaattgtgttgacgcagtctccaggcaccctgtctttgtctccaggggaaagagccaccctctcctgcagggccagtcagagtgttaccagcacctacttagtctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagcatggtagctcaccgtggacgtttggccaggggaccaaggtggaaatcaaa

SEQ ID NO: 44 - protein, 108 aa, Homo sapiens
EIVLTQSPGTLSLSPGERATLSCRASQSVTSTYLVWYQQKPGQAPRLLIYGASSRATGIPDRFSGSGSGTDFTLTISRLEPEDFAVYYCQQHGSSPWTFGQGTKVEIK

SEQ ID NO: 45 - DNA, 351 bp, Homo sapiens
caggtgcagctgcaggagtcgggcccaggactggtgaagccttcacagaccctgtccctcacctgcactgtctctggtggctccatcagcaatggtggttactactggagctggatccgccagcacccagggaagggcctggagtggattgggtacatcttttacagtgggagcacctactacaacccgtccctcaagagtcgacttaccatatcagtagacacgtctaagaaccagttctccctgaagctgagctctgtgactgccgcggacacggccgtgtattactgtgcgagggacagtgatggctttgactactggggccagggaaccctggtcaccgtctcctca

SEQ ID NO: 46 - protein, 117 aa, Homo sapiens
QVQLQESGPGLVKPSQTLSLTCTVSGGSISNGGYYWSWIRQHPGKGLEWIGYIFYSGSTYYNPSLKSRLTISVDTSKNQFSLKLSSVTAADTAVYYCARDSDGFDYWGQGTLVTVSS

SEQ ID NO: 47 - DNA, 324 bp, Homo sapiens
gaaattgtgttgacgcagtctccaggcaccctgtctttgtctccaggggaaagaaccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtttggtagctcaccgctcactttcggcggagggaccaaggtggagatcaaa

SEQ ID NO: 48 - protein, 108 aa, Homo sapiens
EIVLTQSPGTLSLSPGERTTLSCRASQSVSSSYLAWYQQKPGQAPRLLIYGASSRATGIPDRFSGSGSGTDFTLTISRLEPEDFAVYYCQQFGSSPLTFGGGTKVEIK

SEQ ID NO: 49 - DNA, 352 bp, Homo sapiens
caggtgcagctggtggagtctgggggaggcgtggtccagcctgggaggtccctgagactctcctgtgcagcgtctggattcaccttcagtaactatggcatgcactgggtccgccaggctccaggcaaggggctggagtgggtggcagttatatggcatgatggaagtaataaagactatgcagattccgtgaagggccgattcaccatcgccagagacaattccaagaacacgctgtatctgcaaatgaacagcctgagagccgaggacacggctgtgtattactgtgcgagagagcagtggctggtaattgactactggggccagggaaccctggtcaccgtctcctcag

SEQ ID NO: 50 - protein, 117 aa, Homo sapiens
QVQLVESGGGVVQPGRSLRLSCAASGFTFSNYGMHWVRQAPGKGLEWVAVIWHDGSNKDYADSVKGRFTIARDNSKNTLYLQMNSLRAEDTAVYYCAREQWLVIDYWGQGTLVTVSS

SEQ ID NO: 51 - DNA, 321 bp, Homo sapiens
gacatccagatgacccagtctccatcctccctgtccgcatctgtaggagacagagtcaccatcacttgccgggcgagtcagggcattaggaattatttagcctggtatcagcagaaaccagggaaagttcctaagctcctgatctatgttgcatccactttacagtcaggggtcccatctcggttcagtggcagtggatctgggacagttttcactctcaccatcagcagcctgcagcctgaagatattgcaacttattactgtcaaaagtataacagtgccccgtggacgttcggccaagggaccaaggtggaaatcaaa

SEQ ID NO: 52 - protein, 107 aa, Homo sapiens
DIQMTQSPSSLSASVGDRVTITCRASQGIRNYLAWYQQKPGKVPKLLIYVASTLQSGVPSRFSGSGSGTVFTLTISSLQPEDIATYYCQKYNSAPWTFGQGTKVEIK

SEQ ID NO: 53 - DNA, 360 bp, Homo sapiens
gaggtgcagctggtggagtctgggggaggcttggtaaagccgggggggtcccttagactctcctgtgcagcctctggattcactttcagtaacgcctggatgagctgggtccgccaggctccagggaaggggctggagtgggttggccgtattaaaaggaaaactgatggtgagacaacagactacgctgcacccgtgaaaggcagattcaccatctcaagagatgattcaagaaacacgctgtatctgcaaatgaacagcctgaaaaccgaggacacagccgtatattactgtaccatctacacctactactacggaatggacgtctggggccaagggaccacggtcaccgtctcctcg

SEQ ID NO: 54 - protein, 120 aa, Homo sapiens
EVQLVESGGGLVKPGGSLRLSCAASGFTFSNAWMSWVRQAPGKGLEWVGRIKRKTDGETTDYAAPVKGRFTISRDDSRNTLYLQMNSLKTEDTAVYYCTIYTYYYGMDVWGQGTTVTVSS

SEQ ID NO: 55 - DNA, 321 bp, Homo sapiens
gacatccagatgacccagtctccatcctccctgtctgcatctgtaggagacagagtcaccatcacttgccgggcaagtcagagcattaacaactatttaaattggtatcagcagaaaccagggaaagcccctaagctcctgatctatgctgcctccagtttggaaagtggggtcccatcaaggttcagtggcagtggatctgggacagatttcactctcaccatcagcagtctgcaacctgaagattttgcaacttactactgtcaacagagttacagtacccctccgacgttcggccaagggaccaaggtggaaatcaaa

SEQ ID NO: 56 - protein, 107 aa, Homo sapiens
DIQMTQSPSSLSASVGDRVTITCRASQSINNYLNWYQQKPGKAPKLLIYAASSLESGVPSRFSGSGSGTDFTLTISSLQPEDFATYYCQQSYSTPPTFGQGTKVEIK

SEQ ID NO: 57 - DNA, 354 bp, Homo sapiens
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatgtggtatgatgaaaataataactactatgcagactccgtgaagggccgattcaccatctccagagacaattccaagaacacgctgtatctgcaaatgaacagcctgagagccgaggacacggctgtgtattactgtgcgagagaaagcagtggctggtcctttgactactggggccagggaaacctggtcaccgtctcctca

SEQ ID NO: 58 - protein, 118 aa, Homo sapiens
QVQLVESGGGVVQPGRSLRLSCAASGFTFSSYGMHWVRQAPGKGLEWVAVMWYDENNNYYADSVKGRFTISRDNSKNTLYLQMNSLRAEDTAVYYCARESSGWSFDYWGQGNLVTVSS

SEQ ID NO: 59 - DNA, 330 bp, Homo sapiens
cagtctgccctgactcagcctgcctccgtgtctgggtctcctggacagtcgatcaccatctcctgcactggaaccaccagtgacgttggtggttataactatgtctcctggtaccaacagcacccaggcaaagcccccaaactcatgatttatgaggtcagtaatcggccctcaggggtttctaatcgcttctctggctccaagtctggcaacacggcctccctgaccatctctgggctccaggctgaggacgaggctgattattactgcagctcatatacaagcagcagcactctggtattcggcggagggaccaagctgaccgtccta

SEQ ID NO: 60 - protein, 110 aa, Homo sapiens
QSALTQPASVSGSPGQSITISCTGTTSDVGGYNYVSWYQQHPGKAPKLMIYEVSNRPSGVSNRFSGSKSGNTASLTISGLQAEDEADYYCSSYTSSSTLVFGGGTKLTVL

SEQ ID NO: 61 - DNA, 354 bp, Homo sapiens
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtgatatatactgtgcagactccgtgaagggccgattcaccatctccagagacaattccaagaacacgctgtatctgcaaatgaacagcctgcgagccgaggacacggctgtgtattactgtgcgagagagagcagtggctggacttttgactactggggccagggaaccctggtcaccgtctcctca

SEQ ID NO: 62 - protein, 118 aa, Homo sapiens
QVQLVESGGGVVQPGRSLRLSCAASGFTFSSYGMHWVRQAPGKGLEWVAVIWYDGSDIYCADSVKGRFTISRDNSKNTLYLQMNSLRAEDTAVYYCARESSGWTFDYWGQGTLVTVSS

SEQ ID NO: 63 - DNA, 330 bp, Homo sapiens
cagtctgccctgactcagcctgcctccgtgtctgggtctcctggacagtcgatcaccatctcctgcactggaaccagcagtgacgttggtggttataactatgtctcctggtaccaacagcacccaggcaaagcccccaaactcatgatttatgaggtcagtactcggccctcaggggtttctaatcgcttctctggctccaagtctggcaacacggcctccctgaccatctctgggctccaggctgaggacgaggctgattattactgcacctcatatacaagcagcagcactctggtattcggcggagggaccaagctgaccgtccta

SEQ ID NO: 64 - protein, 110 aa, Homo sapiens
QSALTQPASVSGSPGQSITISCTGTSSDVGGYNYVSWYQQHPGKAPKLMIYEVSTRPSGVSNRFSGSKSGNTASLTISGLQAEDEADYYCTSYTSSSTLVFGGGTKLTVL

SEQ ID NO: 65 - DNA, 375 bp, Homo sapiens
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggcagggtcaccatgaccagggacacgtccatcagcacagcctacatggagctgagcaggctgagatctgacgacacggccgtgtattactgtgcgagaggggggccatattgtggtggtgactgctactctgatgcttttgctatctggggccaagggacactggtcaccgtctcctca

SEQ ID NO: 66 - protein, 125 aa, Homo sapiens
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARGGPYCGGDCYSDAFAIWGQGTLVTVSS

SEQ ID NO: 67 - DNA, 333 bp, Homo sapiens
cagtctgtgctgacgcagccgccctcagtgtctggggccccagggctgagggtcaccatctcctgcactgggagcagctccaacatcggggcaggttatgatgtacactggtaccagcagcttccaggaacagcccccaaactcctcatctttgataacagcaatcggccctcaggggtccctgaccgattctctggctccaagtctggcacctcagcctccctggccatcactgggctccaggctgaggatgaggctgattattactgccagtcctatgacagcagcctgcgtggttcggtgttcggcggagggaccaagctgaccgtccta

SEQ ID NO: 68 - protein, 111 aa, Homo sapiens
QSVLTQPPSVSGAPGLRVTISCTGSSSNIGAGYDVHWYQQLPGTAPKLLIFDNSNRPSGVPDRFSGSKSGTSASLAITGLQAEDEADYYCQSYDSSLRGSVFGGGTKLTVL

SEQ ID NO: 69 - DNA, 369 bp, Homo sapiens
caggtgcagctgcaggagtcgggcccaggactggtgaagccttcacagaccctgtccctcacctgcactgtctctggtggctccatcagcagtggtggctactactggagctggatccgccagcacccagggaagggcctggagtggattgggtacatctattacattgggagcacctactacaacccgtccctcaagagtcgagttatcatatcaggagacacgtctaagaaccagttctccctgaagctgagctctgtgactgccgcggacacggccgtgtattactgtgcgagagccgccatctacggtgactaccttgatgcttttgatatctggggccaagggacaatggtcaccgtctcttca

SEQ ID NO: 70 - protein, 123 aa, Homo sapiens
QVQLQESGPGLVKPSQTLSLTCTVSGGSISSGGYYWSWIRQHPGKGLEWIGYIYYIGSTYYNPSLKSRVIISGDTSKNQFSLKLSSVTAADTAVYYCARAAIYGDYLDAFDIWGQGTMVTVSS

SEQ ID NO: 71 - DNA, 324 bp, Homo sapiens
tcttctgagctgactcaggaccctgctgtgtctgtggccttgggacagacagtcaggatcacatgccaaggagacagcctcagaagctattatgtaagctggtgccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgttactcccgggacagcagtggtaaccatcttgtcttcggaactgggaccaaggtcaccgtccta

SEQ ID NO: 72 - protein, 108 aa, Homo sapiens
SSELTQDPAVSVALGQTVRITCQGDSLRSYYVSWCQQKPGQAPVLVIYGKNNRPSGIPDRFSGSSSGNTASLTITGAQAEDEADYYCYSRDSSGNHLVFGTGTKVTVL

SEQ ID NO: 73 - DNA, 371 bp, Homo sapiens
caggtgcagctgcaggagtcgggcccaggactggtgaagccttcggagaccctgtccctcacctgcactgtctctggtggctccatcagtagttactactggagctggatccggcagcccccagagaagggactggagtggattgggtatatctattacagtgggagcaccaaatataatccctccctcaagagtcgagtcaccatatcagtagacacgtccaagaaccagttctccctgaagctgagctctgtgaccgctgcggacacggccgtgtattactgtgcgagagctggagcatattgtggtggtgactgctatcggatgcttttgaaatctggggccaagggacaatggtcaccgtctcttca

SEQ ID NO: 74 - protein, 124 aa, Homo sapiens
QVQLQESGPGLVKPSETLSLTCTVSGGSISSYYWSWIRQPPEKGLEWIGYIYYSGSTKYNPSLKSRVTISVDTSKNQFSLKLSSVTAADTAVYYCARAGAYCGGDCYSDAFEIWGQGTMVTVSS

SEQ ID NO: 75 - DNA, 338 bp, Homo sapiens
cagtctgtgttgacgcagccgccctcagtgtctgcggccccaggacagaaggtcaccatctcctgctctggaagcagctccaacattgggaataattatgtatcctggtaccagcagctcccaggaacagcccccaaactcctcatttatgacaataataagcgaccctcagggattcctgaccgattctctggctccaagtctggcacgtcagccaccctgggcatcaccggattccagactggggacgaggccgattatcactgcggaacatgggaaagaagcctgagtgctggttatgtcttcggaactgggaccaaggtcaccgtcctaggtca

SEQ ID NO: 76 - protein, 112 aa, Homo sapiens
QSVLTQPPSVSAAPGQKVTISCSGSSSNIGNNYVSWYQQLPGTAPKLLIYDNNKRPSGIPDRFSGSKSGTSATLGITGFQTGDEADYHCGTWERSLSAGYVFGTGTKVTVLG

SEQ ID NO: 77 - DNA, 375 bp, Homo sapiens
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggcagggtcaccatgaccagggacacgtccatcagcacagcctacatggagctgagcaggctgagatctgacgacacggccgtgtattactgtgcgagaggggggccatattgtggtggtgactgctactctgatgcttttgctatctggggccaagggacactggtcaccgtctcttca

SEQ ID NO: 78 - protein, 125 aa, Homo sapiens; identical to SEQ ID NO: 66

SEQ ID NO: 79 - DNA, 333 bp, Homo sapiens; identical to SEQ ID NO: 67

SEQ ID NO: 80 - protein, 111 aa, Homo sapiens; identical to SEQ ID NO: 68

SEQ ID NO: 81 - DNA, 351 bp, Homo sapiens
gaggtgcagctggtggagtctgggggaggcttggtccagcctggggggtccctgagactctcctgtgcagcctctggattcacctttagtagcttttggatgaactgggtccgccaggctccagggaaggggctggagtgggtggccaacataaagcaagatggaagtgagaaatactatgtggactctgtgaagggccgattcaccatctccagagacaacgccaagaactcactgtatctgcaaatgaacagcctgagagtcgaggacacgtctgtgtattactgtgcgagagatagggaggtaacattggactactggggccagggaaccctggtcaccgtctcctca

SEQ ID NO: 82 - protein, 117 aa, Homo sapiens
EVQLVESGGGLVQPGGSLRLSCAASGFTFSSFWMNWVRQAPGKGLEWVANIKQDGSEKYYVDSVKGRFTISRDNAKNSLYLQMNSLRVEDTSVYYCARDREVTLDYWGQGTLVTVSS

SEQ ID NO: 83 - DNA, 330 bp, Homo sapiens
cagtctgccctgactcagcctgcctccgtgtctgggtctcctggacagtcgatcaccatctcctgcactggaaccagcagtgatgttgggagttataaccttgtctcctggtaccaacaccacccaggcaaagcccccaaactcatgatttatgacgtcagtaagcggccctcaggggtttctaatcgcttctctggctccaagtctggcaacacggcctccctgacaatctctgggctccaggctgaggacgaggctgattattactgctgctcatatgcaggtagtaacacttgggtgttcggcggagggaccaagctgaccgtccta

SEQ ID NO: 84 - protein, 110 aa, Homo sapiens
QSALTQPASVSGSPGQSITISCTGTSSDVGSYNLVSWYQHHPGKAPKLMIYDVSKRPSGVSNRFSGSKSGNTASLTISGLQAEDEADYYCCSYAGSNTWVFGGGTKLTVL

SEQ ID NO: 85 - DNA, 354 bp, Homo sapiens
caggtgcagctggtggagtctgggggaggcgtggtccagcctgggaggtccctgagactctcctgtgcagcgtctggattcgccttcagtagctatggcatgcactgggtccgccaggctccaggcaaggggctggagtgggtggcagttctatggtatgatggaagtaatatatactgtgcagactccgtgaagggccgattcaccatctccagagacaattccaagaacacgctgtatctgcaaatgaacagcctgagagccgaagacacggctgtgtattactgtgcgagagagagcagtggctggtcctttgactactggggccagggaaccctggtcaccgtctcctca

SEQ ID NO: 86 - protein, 118 aa, Homo sapiens
QVQLVESGGGVVQPGRSLRLSCAASGFAFSSYGMHWVRQAPGKGLEWVAVLWYDGSNIYCADSVKGRFTISRDNSKNTLYLQMNSLRAEDTAVYYCARESSGWSFDYWGQGTLVTVSS

SEQ ID NO: 87 - DNA, 330 bp, Homo sapiens
cagtctgccctgactcagcctgcctccgtgtctgggtctcctggacagtcgatcaccatctcctgcactggaaccagcagtgacgttggtggttataactatgtctcctggtaccaacagcacccaggcaaagcccccaaactcatgatttatgaggtcagtaatcggccctcaggggtttctaatcgcttctctggctccaagtctggcaacacggcctccctgaccatctctgggctccaggctgaggacgaggctgattattactgcggctcatatacaatcagcagcactctggtattcggcggagggaccaagctgaccgtccta

SEQ ID NO: 88 - protein, 110 aa, Homo sapiens
QSALTQPASVSGSPGQSITISCTGTSSDVGGYNYVSWYQQHPGKAPKLMIYEVSNRPSGVSNRFSGSKSGNTASLTISGLQAEDEADYYCGSYTISSTLVFGGGTKLTVL

SEQ ID NO: 89 - DNA, 381 bp, Homo sapiens
caggtgcagctggtggagtctgggggaggcgtggtccagcctgggaggtccctgagactctcctgtgcagcgtctggattcaccttcagtagttatggcatgcactgggtccgccaggctccaggcaaggggctggagtgggtggcagttatatggtatgatggaagtaataaatattatgcagactccgtgaagggccgattcaccatctccagagacaattccaagaacacgctgtatctgcaaatgaacagcctgagagccgaggacacggctgtgtattactgtgcgagagagcatactatggttcggggagttattaactcttactactacggtatggacgtctggggccaagggaccacggtcaccgtctcctca

SEQ ID NO: 90 - protein, 127 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAREHTMVRGVINSYYYGMDVWGQGTTVTVSS

SEQ ID NO: 91 - DNA, 318 bp, Homo sapiens
tcctatgagctgactcagccaccctcagtgtccgtgtccccaggacagacagccagcatcacctgctctggagataaattggggaataaatatgcttgctggtatcagcagaagccaggccagtcccctgtactggtcatctatcaagatagcaagcggccctcagggatccctgagcgattctctggctccaactctgggaacacagccactctgaccatcagcgggacccaggctatggatgaggctgactattactgtcaggcgtgggacagcagcactgtggtattcggcggagggaccaagctgaccgtccta

SEQ ID NO: 92 - protein, 106 aa, Homo sapiens
SYELTQPPSVSVSPGQTASITCSGDKLGNKYACWYQQKPGQSPVLVIYQDSKRPSGIPERFSGSNSGNTATLTISGTQAMDEADYYCQAWDSSTVVFGGGTKLTVL

SEQ ID NO: 93 - DNA, 380 bp, Homo sapiens
caggtgcagctggtgcagtctggggctgaggtgaagaagcctggggcctcagtgaaggtctcctgcaaggcttctggatacaccttcaccagttatgatatcaactgggtgcgacaggccactggacaagggcttgagtggatgggatggatgaaccctaacagtggtaacacaggctatgcacagaagttccagggcagagtcaccatgaccaggaacacctccataagcacagcctacatggagctgagcagcctgagatctgaggacacggccgtgtattactgtgcgagagggtatagcagcagctggtacagggggtactactactactacggtatggacgtctggggccaagggaccacggtcaccgtctcctc

SEQ ID NO: 94 - protein, 126 aa, Homo sapiens
QVQLVQSGAEVKKPGASVKVSCKASGYTFTSYDINWVRQATGQGLEWMGWMNPNSGNTGYAQKFQGRVTMTRNTSISTAYMELSSLRSEDTAVYYCARGYSSSWYRGYYYYYGMDVWGQGTTVTVS

SEQ ID NO: 95 - DNA, 333 bp, Homo sapiens
  misc_feature 85, 86, 87: n = A,T,C or G
gatattgtgatgacccagactccactctctctgtccgtcacccctggacagccggcctccatctcctgcaagtctagtcagagcnnnctgcatagtgctggaaagacctatttgtattggtacctgcagaagccaggccagcctccacagctcctgatctatgaagtttccaaccggttctctggagtgccagataggttcagtggcagcgggtcagggacagatttcacactgaaaatcagccgggtggaggctgaggatgttggggtttattactgcatgcaaaatatacagcttatgtgcagttttggccaggggaccaagctggagatc

SEQ ID NO: 96 - protein, 111 aa, Homo sapiens
  VARIANT 29: Xaa = Any Amino Acid
DIVMTQTPLSLSVTPGQPASISCKSSQSXLHSAGKTYLYWYLQKPGQPPQLLIYEVSNRFSGVPDRFSGSGSGTDFTLKISRVEAEDVGVYYCMQNIQLMCSFGQGTKLEI

SEQ ID NO: 97 - DNA, 354 bp, Homo sapiens
caggtgcagttggtggagtctgggggaggcgtggtccagcctgggaggtccctgagactctcctgtgcagcgtctggattcaccttcagtagctatggcatacactgggtccgccaggctccaggcaaggggctggagtgggtggcagttatctggtatgatggaagtgatatatactatgcagactccgtgaagggccgattcaccatctccagagacaattccaagaacacgctgtatctgcaaatgaacagcctgagagccgaggacacggctctgtattactgtgcgagagagagcagtggctggacctttgactactggggccagggaaccctggtcaccgtctcctca

SEQ ID NO: 98 - protein, 118 aa, Homo sapiens
QVQLVESGGGVVQPGRSLRLSCAASGFTFSSYGIHWVRQAPGKGLEWVAVIWYDGSDIYYADSVKGRFTISRDNSKNTLYLQMNSLRAEDTALYYCARESSGWTFDYWGQGTLVTVSS

SEQ ID NO: 99 - DNA, 330 bp, Homo sapiens
cagtctgccctgactcagcctgcctccgtgtctgggtctcctggacagtcgatcaccatctcctgcactggaaccagcagtgacgttggtggttataactatgtctcctggtaccaacagcacccaggcaaagcccccaaactcatgatttatgaggtcagtaatcggccctcagggatttctaatcgcttctctggctccaagtctggcaacacggcctccctgaccatctctgggctccaggctgaggacgaggctgattattactgcagctcatatacaagcagcagcactataatattcggcggagggaccaagctgaccgtccta

SEQ ID NO: 100 - protein, 110 aa, Homo sapiens
QSALTQPASVSGSPGQSITISCTGTSSDVGGYNYVSWYQQHPGKAPKLMIYEVSNRPSGISNRFSGSKSGNTASLTISGLQAEDEADYYCSSYTSSSTIIFGGGTKLTVL

SEQ ID NO: 101 - DNA, 369 bp, Homo sapiens
caggtgcagctgcaggagtcgggcccaggactggtgaagccttcggagaccctgtccctcacctgcactgtctctggtggctccatcagtagttcctactggagctggatccggcagtccgccgggaagggactggagtggattgggcgtatctatagcagtgggagcaccaactacaatccctccctcaagagtcgagtcaccatgtcagcagacacgtccaagaaccagttctccctgaagctgagctctgtgaccgccgcggacacggccgtgtattactgtgcgagagaaggcagctcgtcgaggtaccactactactacggtatggacgtctggggcccagggaccacggtcaccgtctcctca

SEQ ID NO: 102 - protein, 123 aa, Homo sapiens
QVQLQESGPGLVKPSETLSLTCTVSGGSISSSYWSWIRQSAGKGLEWIGRIYSSGSTNYNPSLKSRVTMSADTSKNQFSLKLSSVTAADTAVYYCAREGSSSRYHYYYGMDVWGPGTTVTVSS

SEQ ID NO: 103 - DNA, 324 bp, Homo sapiens
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcactattcactttcggccctgggaccaaactggatatcaaa

SEQ ID NO: 104 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSLFTFGPGTKLDIK

SEQ ID NO: 105 - DNA, 351 bp, Homo sapiens
caggtgcacctgcaggagtcgggcccaggactggtgaagccttcacagaccctgtccctcacctgcactgtctctggtggctccatcagcagtggtaattactactggagctggatccgccagcacccagggaagggcctggagtggattgggtacatcttttacagtggggacacctactacaatccgtccctcaagagtcgagtaaccatagcagtagacacgtctaagaaccagttctccctgaagctgagttctgtgactgccgcggacacggccgtgtattactgtgcgagagatggctacggtatggacgtctggggccaagggaccacggtcaccgtctcctca

SEQ ID NO: 106 - protein, 117 aa, Homo sapiens
QVHLQESGPGLVKPSQTLSLTCTVSGGSISSGNYYWSWIRQHPGKGLEWIGYIFYSGDTYYNPSLKSRVTIAVDTSKNQFSLKLSSVTAADTAVYYCARDGYGMDVWGQGTTVTVSS

SEQ ID NO: 107 - DNA, 324 bp, Homo sapiens
gacatccagatgacccagtctccatcctccctgtctgcatctgtaggagacagagtcaccatcccttgccgggcaagtcaggacattagaaatgatttaggctggtatcaacagaaaccagggaaagcccctaagcgcctgatctatgctgcatccagtttgcaaagtggggtcccatcacggttcagcggcagtggatctgggacagaattcactctcacaatcagcagcctgcagcctgaagattttgcaacttattactgtctacagcataatagttaccctactctcactttcggcggagggaccaaggtggagatcaaa

SEQ ID NO: 108 - protein, 108 aa, Homo sapiens
DIQMTQSPSSLSASVGDRVTIPCRASQDIRNDLGWYQQKPGKAPKRLIYAASSLQSGVPSRFSGSGSGTEFTLTISSLQPEDFATYYCLQHNSYPTLTFGGGTKVEIK

SEQ ID NO: 109 - DNA, 372 bp, Homo sapiens
caggtgcagctgcaggagtcgggcccaggactggtgaagccttcggagaccctgtccctcacctgcactgtctctggtgtctccatcagtagttactactggagctggatccggcagcccgccgggaagggactggagtggattgggcgtatctattccagtgggagcaccaactacaacccctccctcaagagtcgagtcaccatgtcagtagacacgtccaagaaccagttgtccctgaagctgagctctgtgaccgccgcggacacggccgtgtattactgtgcgagagaggggtatagcagcccgagatacgactactactacggtatggacgtctggggccaagggaccacggtcaccgtctcctca

SEQ ID NO: 110 - protein, 124 aa, Homo sapiens
QVQLQESGPGLVKPSETLSLTCTVSGVSISSYYWSWIRQPAGKGLEWIGRIYSSGSTNYNPSLKSRVTMSVDTSKNQLSLKLSSVTAADTAVYYCAREGYSSPRYDYYYGMDVWGQGTTVTVSS

SEQ ID NO: 111 - DNA, 324 bp, Homo sapiens
gaaattgtgttgacgcagtctccaggcaccctgtctttgtctccaggggaaagagccaccctctcctgcagggccagtcagagtgttaccagcagctacttagcctggtaccagcagaaacctggccaggctcccaggctcctcatctatggtgcatccagcagggccactggcatcccagacaggttcagtggcagtgggtctgggacagacttcactctcaccatcagcggactggagcctgaagattttgcagtgtattactgtcagcagtatggtagctcaatattcactttcggccctgggaccaaagtggttatcaaa

SEQ ID NO: 112 - protein, 108 aa, Homo sapiens
EIVLTQSPGTLSLSPGERATLSCRASQSVTSSYLAWYQQKPGQAPRLLIYGASSRATGIPDRFSGSGSGTDFTLTISGLEPEDFAVYYCQQYGSSIFTFGPGTKVVIK

SEQ ID NO: 113 - DNA, 377 bp, Homo sapiens
caggtgcagctgcaggagtcgggcccaggactggtgaagccttcggagaccctgtccctcacctgcactgtctctggtgtctccatcagtagttactactggagctggatccggcagtccgccgggaagggactggagtggattgggcgtatctataccagtgggagcaccaactacaacccctccctcaagagtcgagtcaccatgtcagtagacacgtccaagaaccagctctccctgaagctgagctctgtgaccgccgcggacacggccgtatattactgtgggagagaagggtactatgaaagtagtggtcattacaactactactacggtatggacgtctggggccacgggaccacggtcaccgtctcctc

SEQ ID NO: 114 - protein, 125 aa, Homo sapiens
QVQLQESGPGLVKPSETLSLTCTVSGVSISSYYWSWIRQSAGKGLEWIGRIYTSGSTNYNPSLKSRVTMSVDTSKNQLSLKLSSVTAADTAVYYCGREGYYESSGHYNYYYGMDVWGHGTTVTVS

SEQ ID NO: 115 - DNA, 324 bp, Homo sapiens
gaaattgtgttgacgcagtctccaggcaccctgtctttgtctccaggggaaaaagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagtttactattcactttcggccctgggaccaaagtggatatcaaa

SEQ ID NO: 116 - protein, 108 aa, Homo sapiens
EIVLTQSPGTLSLSPGEKATLSCRASQSVSSSYLAWYQQKPGQAPRLLIYGASSRATGIPDRFSGSGSGTDFTLTISRLEPEDFAVYYCQQYGSLLFTFGPGTKVDIK

SEQ ID NO: 117 - DNA, 351 bp, Homo sapiens
caggttcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgttcacaatggtaacacaaactatgcacagaaggtccagggcagagtcaccatgaccacagacacatccacgagcacagcctacatggagctgaggagcctgagatctgacgacacggccgtgtattactgtgcgagagacactttctactactttggatactgggtccagggaaccctggtcaccgtctcctca

SEQ ID NO: 118 - protein, 117 aa, Homo sapiens
QVQLVQSGAEVKKPGASVKVSCKASGYTFTSYGISWVRQAPGQGLEWMGWISVHNGNTNYAQKVQGRVTMTTDTSTSTAYMELRSLRSDDTAVYYCARDTFYYFGYWVQGTLVTVSS

SEQ ID NO: 119 - DNA, 319 bp, Homo sapiens
gaaattgtgttgacgcagtctccaggcaccctgtctttgtctccaggggaaagagccaccctctcctgcagggccagtcagagttttagcagcacctacttagcctggtaccagcagaaacctggccaggctcccaggctcctcatctacggtgcatccagcagggccactggcatcccagacaggttcagtggcagtgggtctgggacagacttcactctcaccatcagcagactggagcctgaagattttgcagtgtattactgtcaggagtatggtagctcacctttcggcggagggaccaaggtggagatcaaac

SEQ ID NO: 120 - protein, 106 aa, Homo sapiens
EIVLTQSPGTLSLSPGERATLSCRASQSFSSTYLAWYQQKPGQAPRLLIYGASSRATGIPDRFSGSGSGTDFTLTISRLEPEDFAVYYCQEYGSSPFGGGTKVEIK

SEQ ID NO: 121 - DNA, 366 bp, Homo sapiens
gaggtgcagctggtggagtctgggggaggcttggtaaagccagggcggtccctgagactctcctgtacagcttctggaatcacctttggtgattatgctttgagctggttccgccaggctccagggaaggggctggagtgggtaggtttcattagaagcgaagcttatggtgggacaacagaatacgccgcgtctgtgaaaggcagattcaccatctcaagagatgattccaaaagcatcgcctatctgcaaatgaacagcctgaaaaccgaggacacagccgtgtattactgtactagaggtggatacagctatggttacgactttgactactggggccagggaaccctggtcaccgtctcctca

SEQ ID NO: 122 - protein, 122 aa, Homo sapiens
EVQLVESGGGLVKPGRSLRLSCTASGITFGDYALSWFRQAPGKGLEWVGFIRSEAYGGTTEYAASVKGRFTISRDDSKSIAYLQMNSLKTEDTAVYYCTRGGYSYGYDFDYWGQGTLVTVSS

SEQ ID NO: 123 - DNA, 336 bp, Homo sapiens
gatattttgatgacccagactccactctctctgtccgtcacccctggacagccggcctccatctcctgcaagtctagtcagcgcctcctgcatagtgatggaaagacctatttgtcttggtacctccagaagccaggccagcctccacagctcctgatctatgaagtttccaaccggttctctggagtgccagataggttcagtggcagcgggtcagggacagatttcacactgaaaatcagccgggtggaggctgaggatgttggggtttattactgcatgcaaagtatacaggttcctctcactttcggcggagggaccaaggtggagatcaaa

SEQ ID NO: 124 - protein, 112 aa, Homo sapiens
DILMTQTPLSLSVTPGQPASISCKSSQRLLHSDGKTYLSWYLQKPGQPPQLLIYEVSNRFSGVPDRFSGSGSGTDFTLKISRVEAEDVGVYYCMQSIQVPLTFGGGTKVEIK

SEQ ID NO: 125 - DNA, 375 bp, Homo sapiens
caggtgcagctgcaggagtcgggcccaggactggtgaagccttcggagaccctgtccctcacctgcactgtctctggtggctccatcagtagtaactactggagctggatccggcagtccgccgggaagggactggagtggattgggcgtatctataccagtgggagcaccaactacaacccctccctcaagagtcgagtcaccatgtcagtagacacgtccaagaaccagttctccctgaagttgaactctgtgaccgccgcggacacggccgtgtattactgtgcgagagaggggtatagcagctcgtcggggtactactactactacggtttggacgtctggggccaagggaccacggtcaccgtctcctca

SEQ ID NO: 126 - protein, 125 aa, Homo sapiens
QVQLQESGPGLVKPSETLSLTCTVSGGSISSNYWSWIRQSAGKGLEWIGRIYTSGSTNYNPSLKSRVTMSVDTSKNQFSLKLNSVTAADTAVYYCAREGYSSSSGYYYYYGLDVWGQGTTVTVSS

SEQ ID NO: 127 - DNA, 328 bp, Homo sapiens
cagtctgtgctgactcagccaccctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtggactccagtctgaggatgaggctgattattactgtgcagcatgggatgacagcctgaatggtggtattcggcggagggaccaagctgaccgtccta

SEQ ID NO: 128 - protein, 109 aa, Homo sapiens
QSVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGGIRRRDQADRP

SEQ ID NO: 129 - DNA, 381 bp, Homo sapiens
caggtgcagctggtggagtctgggggaggcgtggtccagcctgggaggtccctgagactctcctgtgcagcgtctggattcaccttcagtacctatggcatgcactgggtccgccaggctccaggcaaggggctggagtgggtggcagttatatggtatgatggaagtaagaaatactatgcagactccgtgaagggccgattcgccatctccagagacaattccaagaacacgctgtatctgcaaatgaacagcctgagagccgaggacacggctgtgtattactgtgcgagagatcatactatggttcggggagttattaactcctactactacggtatggccgtctggggccaagggaccacggtcaccgtctcctca

SEQ ID NO: 130 - protein, 127 aa, Homo sapiens
QVQLVESGGGVVQPGRSLRLSCAASGFTFSTYGMHWVRQAPGKGLEWVAVIWYDGSKKYYADSVKGRFAISRDNSKNTLYLQMNSLRAEDTAVYYCARDHTMVRGVINSYYYGMAVWGQGTTVTVSS

SEQ ID NO: 131 - DNA, 318 bp, Homo sapiens
tcctatgagctgactcagccaccctcagtgtccgtgtccccaggacagacagccagcatcacctgctctggagatagattggggaataaatatgcttgctggtatcagcagaagacaggccagtcccctgtgttggtcatctatcaagatagcaagcggccctcagggatccctgagcgattctctggctccaactctgggaacacagccactctgaccatcagcgggacccaggctatggatgaggctgactattactgtcaggcgtgggacagcagcactgtggtattcggcggagggaccaagctgaccgtccta

SEQ ID NO: 132 - protein, 106 aa, Homo sapiens
SYELTQPPSVSVSPGQTASITCSGDRLGNKYACWYQQKTGQSPVLVIYQDSKRPSGIPERFSGSNSGNTATLTISGTQAMDEADYYCQAWDSSTVVFGGGTKLTVL

SEQ ID NO: 133 - DNA, 380 bp, Homo sapiens
caggtgcagctgcaggagtcgggcccaggactggtgaagccttcggagaccctgtccctcacctgcactgtctctggtggctccgtcagcagtggtggttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcgagaggggggaatgactacagtaactacgaagactactactacggtatggacgtctggggccaagggaccacggtcaccgtctcctc

SEQ ID NO: 134 - protein, 126 aa, Homo sapiens
QVQLQESGPGLVKPSETLSLTCTVSGGSVSSGGYYWSWIRQPPGKGLEWIGYIYYSGSTNYNPSLKSRVTISVDTSKNQFSLKLSSVTAADTAVYYCARGGNDYSNYEDYYYGMDVWGQGTTVTVS

SEQ ID NO: 135 - DNA, 330 bp, Homo sapiens
cagtctgccctgactcagcctgcctccgtgtctgggtctcctggacagtcgatcaccatctcctgcactggaaccagcagtgatgttgggagttataaccttgtctcctggtaccaacagcacccaggcaaagcccccaaactcatgatttatgaggtcagtaagcggccctcaggggtttctaatcgcttctctggctccaagtctggcaacacggcctccctgacaatctctgggctccaggctgaggacgaggctgaatattactgctgctcatatgcaggtagaagcactttggtattcggcggagggaccaaactgaccgtccta

SEQ ID NO: 136 - protein, 110 aa, Homo sapiens
QSALTQPASVSGSPGQSITISCTGTSSDVGSYNLVSWYQQHPGKAPKLMIYEVSKRPSGVSNRFSGSKSGNTASLTISGLQAEDEAEYYCCSYAGRSTLVFGGGTKLTVL

SEQ ID NO: 137 - DNA, 389 bp, Homo sapiens
caggtgcagctgcaggagtcgggcccaggactggtgaagccttcacagaccctgtccctcacctgcactgtctctggtggctccatcagcagtggtgattactactggagctggatccgccagcacccagggaagggcctggagtggattgggtacatctattacagtgggagcacctactacaacccgtccctcaagagtcgacttaccatatcagtagacacgtctaagaaccagttctccctgaagctgagctctgtgactgccgcggacacggccgtgttttactgtgcgagagggggacgtattactatggttcggggagttattccccactactactacggtatggacgtctggggccaagggaccacggtcaccgtctcctc

SEQ ID NO: 138 - protein, 129 aa, Homo sapiens
QVQLQESGPGLVKPSQTLSLTCTVSGGSISSGDYYWSWIRQHPGKGLEWIGYIYYSGSTYYNPSLKSRLTISVDTSKNQFSLKLSSVTAADTAVFYCARGGRITMVRGVIPHYYYGMDVWGQGTTVTVS

SEQ ID NO: 139 - DNA, 318 bp, Homo sapiens
tcctatgagctgactcagccaccctcagtgtccgtgtccccaggacagacagccagcatcacctgctctggagataaattgggggttaaatatgcttgctggtatcagcagaagccaggccagtcccctgtgctggtcatctatcaagatagcaagcggccctcagggatccctgagcgattctctggctccaacgctgggaacacagccactctgaccatcagcgggactcaggctatggatgaggctgactattactgtcagacgggggacagcagcactgtggtattcggcggagggaccaagctgaccgtccta

SEQ ID NO: 140 - protein, 106 aa, Homo sapiens
SYELTQPPSVSVSPGQTASITCSGDKLGVKYACWYQQKPGQSPVLVIYQDSKRPSGIPERFSGSNAGNTATLTISGTQAMDEADYYCQTGDSSTVVFGGGTKLTVL

SEQ ID NO: 141 - DNA, 384 bp, Homo sapiens
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggctgtgtattactgtgcgagagaccggagtactatggttcggggagttattatcccctactactacggtatggacgtctggggccaagggaccacggtcaccgtctcctca

SEQ ID NO: 142 - protein, 128 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDRSTMVRGVIIPYYYGMDVWGQGTTVTVSS

SEQ ID NO: 143 - DNA, 318 bp, Homo sapiens
tcctatgagctgactcagccaccctcagtgtccgtgtccccaggacagacagccagcatcacctgctctggagataaattggggaataaatatgcttgctggtatcagcagaagccaggccagtcccctgtgcaggtcatctatcaagataccaagcggccctcagggatccctgagcgattctctggctccaactctgggaacacagccactctgaccatcagcgggacccaggctatggatgaggctgactattactgtcaggcgtgggacagcagcaatgtggtattcggcggagggaccaagctgatcgtccta

SEQ ID NO: 144 - protein, 106 aa, Homo sapiens
SYELTQPPSVSVSPGQTASITCSGDKLGNKYACWYQQKPGQSPVQVIYQDTKRPSGIPERFSGSNSGNTATLTISGTQAMDEADYYCQAWDSSNVVFGGGTKLIVL

SEQ ID NO: 145 - DNA, 354 bp, Homo sapiens
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagatacatggtatgatggaagtgataaatactatgcagattccgtgaagggccgattcaccatctccagagacaattccaagaacacgctgtatctgcaaatgaacagcctgagagccgaggacacggctgtgtattactgtgcgagagaaggccgtggctggtcctttgactactggggccagggaaccctggtcaccgtctcctca

SEQ ID NO: 146 - protein, 118 aa, Homo sapiens
QVQLVESGGGVVQPGRSLRLSCAASGFTFSSYGMHWVRQAPGKGLEWVADTWYDGSDKYYADSVKGRFTISRDNSKNTLYLQMNSLRAEDTAVYYCAREGRGWSFDYWGQGTLVTVSS

SEQ ID NO: 147 - DNA, 330 bp, Homo sapiens
cagtctgccctgactcagcctgcctccgtgtctgggtctcctggacagtcgatcaccatctcctgcactggaaccagcagtgacgttggtggttataactatgtctcctggtaccaacagtacccaggcaaagcccccaaactcatgatttatgaggtcagtaatcggccctcaggggtttctaatcgcttctctggctccaagtctggcaacacggcctccctgaccatctctgggctccaggctgaggacgaggctgattattactgcagctcatatacaagcagcagcactctggtattcggcggagggaccaagctgaccgtccta

SEQ ID NO: 148 - protein, 110 aa, Homo sapiens
QSALTQPASVSGSPGQSITISCTGTSSDVGGYNYVSWYQQYPGKAPKLMIYEVSNRPSGVSNRFSGSKSGNTASLTISGLQAEDEADYYCSSYTSSSTLVFGGGTKLTVL

SEQ ID NO: 149 - DNA, 384 bp, Homo sapiens
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggctgtgtattactgtgcgagagataatggggcgatggttcggggagttattataagctactactacggtatggacgtctggggccaagggaccacggtcaccgtctcctca

SEQ ID NO: 150 - protein, 128 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDNGAMVRGVIISYYYGMDVWGQGTTVTVSS

SEQ ID NO: 151 - DNA, 318 bp, Homo sapiens
tcctatgacctgactcagccatcctcagtgtccgtgtccccaggacagacagccagcatcgcctgctctggagatagattggggaataaatatgcttgctggtatcagcagaagccaggccagtcccctgtgctggtcatctataaagatagcaagcggccctcagggatccctgagcgattctctggctccaattctgggaacacagccactctgaccatcagcgggacccaggctatggatgaggctgactattactgtcaggcgtgggacagcagcactgtggtattcggcggagggaccaagctgaccgtcctg

SEQ ID NO: 152 - protein, 106 aa, Homo sapiens
SYDLTQPSSVSVSPGQTASIACSGDRLGNKYACWYQQKPGQSPVLVIYKDSKRPSGIPERFSGSNSGNTATLTISGTQAMDEADYYCQAWDSSTVVFGGGTKLTVL

SEQ ID NO: 153 - DNA, 372 bp, Homo sapiens
caggtgcagctggtgcagtctggggctgaggtgaagaagcctggggcctcagtgaaggtttcctgcaaggcatctggatacaccttcaccagctactatatgaactgggtgcgacaggcccctggacaagggcttgagtggatgggaataatcagccctagtggtggtagcacaacctacgctcagaagttccagggcagagtcaccatgaccagggacacgtccacgagcacagtctacatggagctgagcagcctgagatctgaggacacggccgtgtattactgtgcgaggactggatatagtggctacgagggatactactacggtttggacgtctggggccaagggaccacggtcaccgtctcctca

SEQ ID NO: 154 - protein, 124 aa, Homo sapiens
QVQLVQSGAEVKKPGASVKVSCKASGYTFTSYYMNWVRQAPGQGLEWMGIISPSGGSTTYAQKFQGRVTMTRDTSTSTVYMELSSLRSEDTAVYYCARTGYSGYEGYYYGLDVWGQGTTVTVSS

SEQ ID NO: 155 - DNA, 336 bp, Homo sapiens
gatgttgtgatgactcagtctccactctcccagcccgtcacccttggacagccggcctccatctcctgcaggtctagtcaaagcctcgtatacagtgatgggaacacctacttgcattggtttcagcagaggccaggccaatctccaaggcgcctaatttataaggtttctaactgggactctggggtcccagacagattcagcggcagtgggtcaggcactgatttcacactgaaaatcagcagggtggaggctgaggatgttggggtttatgactgcatgcaaggtacacactggccctctactttcggcggagggaccaaggtggagatcaaa

SEQ ID NO: 156 - protein, 112 aa, Homo sapiens
DVVMTQSPLSQPVTLGQPASISCRSSQSLVYSDGNTYLHWFQQRPGQSPRRLIYKVSNWDSGVPDRFSGSGSGTDFTLKISRVEAEDVGVYDCMQGTHWPSTFGGGTKVEIK

SEQ ID NO: 157 - DNA, 381 bp, Homo sapiens
caggtgcagctggtggagtctgggggaggcgtggtccagcctgggaggtccctgagactctcctgtgcagcgtctggattcaccttcagaagctatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggctgtgtattactgtgcgagagatagtactatggttcggggaattattaactactactactacggtatggacgtctggggccaagggaccacggtcaccgtctcctca

SEQ ID NO: 158 - protein, 127 aa, Homo sapiens
QVQLVESGGGVVQPGRSLRLSCAASGFTFRSYGMHWVRQAPGKGLEWVAVIWYDGSNKYYADSVKGRFTISRDNSKNTLYLQMNSLRAEDTAVYYCARDSTMVRGIINYYYYGMDVWGQGTTVTVSS

SEQ ID NO: 159 - DNA, 318 bp, Homo sapiens
tcctatgagctgactcagccaccctcagtgtccgtctccccaggacagacagccagcatcacctgctctggagataaattggggaataaatatgctcactggtatcagcagaagccaggccagtcccctgtgctggtcatctatcaagataacaagcggccctcagggatccctgagcgattctctggttccaactctgggaacacagccactctgaccatcagcgggacccaggctatggatgaggctgacttttactgtcaggcgtgtgacagcagctctgtggtattcggcggagggaccaagctgaccgtccta

SEQ ID NO: 160 - protein, 106 aa, Homo sapiens
SYELTQPPSVSVSPGQTASITCSGDKLGNKYAHWYQQKPGQSPVLVIYQDNKRPSGIPERFSGSNSGNTATLTISGTQAMDEADFYCQACDSSSVVFGGGTKLTVL

SEQ ID NO: 161 - DNA, 366 bp, Homo sapiens
caggtgcagctgcaggagtcgggcccaggactggtgaagccttcggagaccctgtccctcacctgcactgtctctggtggctccatcagtagtcactactggagctggatccggcagcccgccgggaagggactggagtggattgggcgtatctctgccagtgggagcacccactacaacccctccctcaagagtcgagtcaccatgtcagtagacacgtccaagaaccagttctccctgaagctgagctctgtgaccgccgcggacacggccgtgtattactgtgcgagagagggtggaaccacctactactactattacggtatggacgtctggggccaagggaccacggtcaccgtctcctca

SEQ ID NO: 162 - protein, 122 aa, Homo sapiens
QVQLQESGPGLVKPSETLSLTCTVSGGSISSHYWSWIRQPAGKGLEWIGRISASGSTHYNPSLKSRVTMSVDTSKNQFSLKLSSVTAADTAVYYCAREGGTTYYYYYGMDVWGQGTTVTVSS

SEQ ID NO: 163 - DNA, 321 bp, Homo sapiens
gaaattgtgttgacgcagtctccaggcaccctgtctttgtctccaggggaaagagccaccctctcctgcagggccagtcagagtgttcgcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattcctgtcagcagtatattaactcattcactttcggccctgggaccaaagtggatatcaat

SEQ ID NO: 164 - protein, 107 aa, Homo sapiens
EIVLTQSPGTLSLSPGERATLSCRASQSVRSSYLAWYQQKPGQAPRLLIYGASSRATGIPDRFSGSGSGTDFTLTISRLEPEDFAVYSCQQYINSFTFGPGTKVDIN

SEQ ID NO: 165 - DNA, 360 bp, Homo sapiens
caggtgcagctggtgcagtctggggctgaggtgaagaagcctggggcctcagtgaaggtctcctgcaaggcttctggatacaccttcaccggctactttatacactgggtgcgacaggcccctggacaagggcttgagtggatgggatggatcaaccctaacagtggtggcacaaactatgcacagaagtttcagggcagggtcaccatgaccagggacacgtccatcaacacagcctacatggagctgagcaggctgagatctgacgacacggccgtgtattactgtgcgagaaacgtattacgatattttgactggttattgcactggggccagggaaccctggtcaccgtctcctca

SEQ ID NO: 166 - protein, 120 aa, Homo sapiens
QVQLVQSGAEVKKPGASVKVSCKASGYTFTGYFIHWVRQAPGQGLEWMGWINPNSGGTNYAQKFQGRVTMTRDTSINTAYMELSRLRSDDTAVYYCARNVLRYFDWLLHWGQGTLVTVSS

SEQ ID NO: 167 - DNA, 339 bp, Homo sapiens
gacatcgtgatgacccagtctccagactccctggctgtgtctctgggcgagagggccaccatcaactgcaagtccagccagagttttttatacagctccaacaataagaactccttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactccgatcaccttcggccaagggacacgactggagattaaa

SEQ ID NO: 168 - protein, 113 aa, Homo sapiens
DIVMTQSPDSLAVSLGERATINCKSSQSFLYSSNNKNSLAWYQQKPGQPPKLLIYWASTRESGVPDRFSGSGSGTDFTLTISSLQAEDVAVYYCQQYYSTPITFGQGTRLEIK

SEQ ID NO: 169 - DNA, 366 bp, Homo sapiens
caggtgcatctgcaggagtcgggcccaggactggtgaagccttcacagaccctgtccctcacctgcactgtctctggtggctccatcagcagtggtggttactactggagctggatccgccagcacccagggaagggcctggagtggattgggtacatctattacagtggaaacacctactacaacccgtccctcaagagtcgagttaccatatcaatagacacgtctaagaaccagttctccctgaagctgagctctgtgactgccgcggacacggccgtgtattactgtgtgagagatcggagcttcggtgactacgggtcgtttgactactggggccagggaaccctggtcaccgtctcctca

SEQ ID NO: 170 - protein, 122 aa, Homo sapiens
QVHLQESGPGLVKPSQTLSLTCTVSGGSISSGGYYWSWIRQHPGKGLEWIGYIYYSGNTYYNPSLKSRVTISIDTSKNQFSLKLSSVTAADTAVYYCVRDRSFGDYGSFDYWGQGTLVTVSS

SEQ ID NO: 171 - DNA, 321 bp, Homo sapiens
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgttgcatcccgtttgcaaggtggggtcccatcaaggttcagtggcagtggatctgggacagatttcactctcaccatcagcagtctgcaacctgaagattttgcaacttactactgtcaacagggttacatttccccattcactttcggccctgggaccaaagtggatatcaaa

SEQ ID NO: 172 - protein, 107 aa, Homo sapiens
DIQMTQSPSSLSASVGDRVTITCRASQSISSYLNWYQQKPGKAPKLLIYVASRLQGGVPSRFSGSGSGTDFTLTISSLQPEDFATYYCQQGYISPFTFGPGTKVDIK

SEQ ID NO: 173 - DNA, 351 bp, Homo sapiens
caggtgcagctgcaggagtcgggcccaggactggtgaagccttcacagaccctgtccctcacctgcactgtctctggtggctccatcagcagtggtaattactactggagctggatccgccagcacccagggaagggcctggagtggattgggtacatctattacagtgggagcacctactacaacccgtccctcaagagtcgaattaccatatcagtagacacgtctaagaaccagttctccctgaagctgagctctgtgactgccgcggacacggccgtgtattactgtgcgagagatggctacggtatggacgtctggggccaagggaccacggtcaccgtctcctca

SEQ ID NO: 174 - protein, 117 aa, Homo sapiens
QVQLQESGPGLVKPSQTLSLTCTVSGGSISSGNYYWSWIRQHPGKGLEWIGYIYYSGSTYYNPSLKSRITISVDTSKNQFSLKLSSVTAADTAVYYCARDGYGMDVWGQGTTVTVSS

SEQ ID NO: 175 - DNA, 324 bp, Homo sapiens
gaaattgtgttgacgcagtctccaggcaccctgtctttgtctccaggggaaagagccaccctctcctgcagggccagtcagagtgttagcagcagctacttagtgtggtaccagcagaaacctggccaggctcccaggctcctcatctatggagcatccagcagggccactggcatcccagacaggttcagtggcagtgggtctgggacagacttcactctcaccatcagcagactggagcctgaagattttgcagtgtattactgtcagcagtatggtagctcaccgctcactttcggcggagggaccaaggtggagatcaaa

SEQ ID NO: 176 - protein, 108 aa, Homo sapiens
EIVLTQSPGTLSLSPGERATLSCRASQSVSSSYLVWYQQKPGQAPRLLIYGASSRATGIPDRFSGSGSGTDFTLTISRLEPEDFAVYYCQQYGSSPLTFGGGTKVEIK

SEQ ID NO: 177 - DNA, 363 bp, Homo sapiens
caggtgcagctgcaggagtcgggcccaggactggtgaagccttcacagaccctgtccctcacctgcactgtctctggtggctccatcagcagtggtggttactactggagctggatccgccagcacccagggaagggcctggagtggattgggtacatctattacagtgggagcacctactacaacccgtccctcaagagtcgagttaccatatcagtagacacgtctaagaaccagttctccctgaagctgagctctgtgactgccgcggacacggccgtgtattactgtgcgagatggatgatagtagactactacggtatggacgtctggggccaagggaccacggtcaccgtctcctca

SEQ ID NO: 178 - protein, 121 aa, Homo sapiens
QVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARWMIVDYYGMDVWGQGTTVTVSS

SEQ ID NO: 179 - DNA, 324 bp, Homo sapiens
gaaattgtgttgacgcagtctccaggcaccctgtctttgtctccaggggaaagagccaccctctcctgcagggccagtcagagtattagcagcagctacttagcctggtaccagcagaaacctggccaggctcccaggctcctcatctatggtgcatccagtagggccactggcatcccagacaggttcagtggcagtgggtctgggacagatttcactctcaccatcagcagactggagcctgaagattttgcagtgtattactgtcagcagtatggtagcgcaatgtgcagttttggccaggggaccaagttggagatcaaa

SEQ ID NO: 180 - protein, 108 aa, Homo sapiens
EIVLTQSPGTLSLSPGERATLSCRASQSISSSYLAWYQQKPGQAPRLLIYGASSRATGIPDRFSGSGSGTDFTLTISRLEPEDFAVYYCQQYGSAMCSFGQGTKLEIK

SEQ ID NO: 181 - DNA, 365 bp, Homo sapiens
caggtgcagctggtggagtctgggggaggcgtggtccagcctgggaggtccctgagactctcctgtgcagcgtctggattcaccttcagaagctatggcatgcactgggtccgccaggctccaggcaaggggctggaatgggtggcaattatatggtatgatggaagtaataaatactatgcagactccgtgaagggccgattcaccatctccagagacaattccaagaacacgcagtatctgcaaatgaacagcctgagagccgaggacacggctgtgtattactgtgcgagaggccagtttagccacgggcactactacggtatggacgtctggggccaagggaccacggtcaccgtctcctc

SEQ ID NO: 182 - protein, 121 aa, Homo sapiens
QVQLVESGGGVVQPGRSLRLSCAASGFTFRSYGMHWVRQAPGKGLEWVAIIWYDGSNKYYADSVKGRFTISRDNSKNTQYLQMNSLRAEDTAVYYCARGQFSHGHYYGMDVWGQGTTVTVS

SEQ ID NO: 183 - DNA, 324 bp, Homo sapiens
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatccccgacaggttcagtggcagtgggtctgggacagacttcactctcaccctcagcagactggagcctgaagattttgcagtgtattactgtcagcagtatggtggctcaccattcactttcggccctgggaccaaagtggatatcaaa

SEQ ID NO: 184 - protein, 108 aa, Homo sapiens
EIVLTQSPGTLSLSPGERATLSCRASQSVSSSYLAWYQQKPGQAPRLLIYGASSRATGIPDRFSGSGSGTDFTLTLSRLEPEDFAVYYCQQYGGSPFTFGPGTKVDIK

SEQ ID NO: 185 - DNA, 374 bp, Homo sapiens
gaggtgcagctggtgcagtctggagaagaggtgaaaaagcccggggagtctctgaggatctcctgtaagggttctggatacagcgttaccagctactggatcggctgggtgcgccagatgcccgggaaaggcctggagtggatgggaatcatctatcccggtgactctgataccagatacagcccgtccttccaaggccaggtcaccatctcagccgacaagtccatcaggaccgcctacctgcagtggagcagtgtgaaggcctcggacaccgccatatatttttgtgcgagattccaaggagattgtagtggtggtagttgctacggacactttgactactggggccagggaaccctggtcatcgtctcctc

SEQ ID NO: 186 - protein, 124 aa, Homo sapiens
EVQLVQSGEEVKKPGESLRISCKGSGYSVTSYWIGWVRQMPGKGLEWMGIIYPGDSDTRYSPSFQGQVTISADKSIRTAYLQWSSVKASDTAIYFCARFQGDCSGGSCYGHFDYWGQGTLVIVS

SEQ ID NO: 187 - DNA, 324 bp, Homo sapiens
gaaattgtgttgacgcagtctccaggcaccctgtctttgtctccaggggaaagagccaccctctcctgcagggccagtcagagtgttagcagcagctacttagcctggtaccagcagaaacctgcccaggctcccaggctcctcatctatgttgcatccaggagggccactggcatccccgacaggttcagtggcagtgggtctgggacagacttcactctcaccatcagcagactggagcctgaagattttgcagtgtattactgtcagcagtatggtagttcatcgtggacgttcggccaagggaccacggtggaaatcaaa

SEQ ID NO: 188 - protein, 108 aa, Homo sapiens
EIVLTQSPGTLSLSPGERATLSCRASQSVSSSYLAWYQQKPAQAPRLLIYVASRRATGIPDRFSGSGSGTDFTLTISRLEPEDFAVYYCQQYGSSSWTFGQGTTVEIK

SEQ ID NO: 189 - DNA, 374 bp, Homo sapiens
gaggtgcagctggtgcagtctggagcagaggtgaaaaagcccggggagtctctgaggatctcctgtaagggttctggatacagcgtttccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgccttcctgcagtggatcagtctgaaggcctcggacaccgccatgtattactgtgcgagactccaaggagattgtagtggtggtagctgctacgggcactttgactactggggccagggaaccctggtcaccgtctcctc

SEQ ID NO: 190 - protein, 124 aa, Homo sapiens
EVQLVQSGAEVKKPGESLRISCKGSGYSVSSYWIGWVRQMPGKGLEWMGIIYPGDSDTRYSPSFQGQVTISADKSISTAFLQWISLKASDTAMYYCARLQGDCSGGSCYGHFDYWGQGTLVTVS

SEQ ID NO: 191 - DNA, 324 bp, Homo sapiens
gaaattgtgttgacgcagtctccaggcaccctgtctttgtctccaggggaaagagccaccctctcctgcagggccagtcagactattagcagcggctacttagcctggtaccaacagaaacctgcccaggctcccaggctcctcatctatgttgcatccaggagggccactggcatccccgacaggttcagtggcagtgggtctgggacagacttcactctcaccatcagcagactggagcctgaagattttgcagtgtattactgtcagcagtatggtagttcatcgtggacgttcggccaagggaccacggtggaaatcaaa

SEQ ID NO: 192 - protein, 108 aa, Homo sapiens
EIVLTQSPGTLSLSPGERATLSCRASQTISSGYLAWYQQKPAQAPRLLIYVASRRATGIPDRFSGSGSGTDFTLTISRLEPEDFAVYYCQQYGSSSWTFGQGTTVEIK

SEQ ID NO: 193 - DNA, 374 bp, Homo sapiens
gaggtgcagctggtgcagtctggagcagaggtgaaaaagcccggggagtctctgaggatctcctgtaagggttctggatacagcgttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagtctgaaggcctcggacaccgccatgtattactgtgcgagactccaaggagattgtagtggtggtacttgctacgggcactttgactactggggccagggaaccctggtcaccgtctcctc

SEQ ID NO: 194 - protein, 124 aa, Homo sapiens
EVQLVQSGAEVKKPGESLRISCKGSGYSVTSYWIGWVRQMPGKGLEWMGIIYPGDSDTRYSPSFQGQVTISADKSISTAYLQWSSLKASDTAMYYCARLQGDCSGGTCYGHFDYWGQGTLVTVS

SEQ ID NO: 195 - DNA, 324 bp, Homo sapiens
gaaattgtgttgacgcagtctccaggcaccctgtctttgtctccaggggaaagagccaccctctcctgcagggccagtcagagtgttagcagcagctacttagcctggtaccagcagaaacctgcccaggctcccaggctcctcatctatgttgcatccaggagggccactggcatccccgacaggttcagtggcagtgggtctgggacagacttcactctcaccatcagcagactggagcctgaagattttgcagtgtattactgtcagcagtatggtacctcatcgtggacgttcggccaagggaccacggtggaaatcaaa

SEQ ID NO: 196 - protein, 108 aa, Homo sapiens
EIVLTQSPGTLSLSPGERATLSCRASQSVSSSYLAWYQQKPAQAPRLLIYVASRRATGIPDRFSGSGSGTDFTLTISRLEPEDFAVYYCQQYGTSSWTFGQGTTVEIK

SEQ ID NO: 197 - DNA, 354 bp, Homo sapiens
caggttcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtcacacaaactatgcacagaagctccagggcagagtcaccatgaccacagacacatccacgagcacagcctacatggaactgaggagcctgagatctgacgacacggccgtgtattactgtgcgagagatagcagcccgtactactttgactactggggccagggaaccctggtcaccgtctcctca

SEQ ID NO: 198 - protein, 118 aa, Homo sapiens
QVQLVQSGAEVKKPGASVKVSCKASGYTFTSYGISWVRQAPGQGLEWMGWISAYNGHTNYAQKLQGRVTMTTDTSTSTAYMELRSLRSDDTAVYYCARDSSPYYFDYWGQGTLVTVSS

SEQ ID NO: 199 - DNA, 315 bp, Homo sapiens
gaaattgtgttgacgcagtctccaggcaccctgtctttgtctccaggggaaagagccaccctctcctgcagggccagtcagagtgttcgcagcacctacttagcctggtaccagcagaaacctggccaggctcccaggctcctcatctatggagcatccagcagggccattggcatcccagacaggttcagtggcagtgggtctgggacagacttcactctcaccatcagaagactggagcctgaagattttgctgtgttttactgtcagcagtatggtagcagttttggccaggggaccaaactggagatcaaa

SEQ ID NO: 200 - protein, 105 aa, Homo sapiens
EIVLTQSPGTLSLSPGERATLSCRASQSVRSTYLAWYQQKPGQAPRLLIYGASSRAIGIPDRFSGSGSGTDFTLTIRRLEPEDFAVFYCQQYGSSFGQGTKLEIK

SEQ ID NO: 201 - DNA, 360 bp, Homo sapiens
caggtgcttctgcaggagtcgggcccaggactggtgaagccttcacagaccctgtccctcacctgcactgtctctggtggctccatcagcagtggtggttattattggagctggatccgccagcacccagggaagggcctggagtggatagggtacatcaattacagtgggagcgcctactacaacccgtccctcaagagtcgagttaccatatcagtagacacgtctaagaaccagttctccctgaagctgagttctgtgacggccgcggacacggccgtgtattactgtgcgagagatcactggttattaagggggtttgactactggggccagggaaccctggtcaccgtctcctca

SEQ ID NO: 202 - protein, 120 aa, Homo sapiens
QVLLQESGPGLVKPSQTLSLTCTVSGGSISSGGYYWSWIRQHPGKGLEWIGYINYSGSAYYNPSLKSRVTISVDTSKNQFSLKLSSVTAADTAVYYCARDHWLLRGFDYWGQGTLVTVSS

SEQ ID NO: 203 - DNA, 336 bp, Homo sapiens
gatattgtaatgactcagactccactctctctgtccgtcacccctggacagccggcctccatctcctgcaagtctagtcagagcctcctgcatagtgatggaaagacctatttgtattggtacctgcagaagccaggccagcctccacagctcctgatctatgaagtttccaaccggttctctggagtgccagataggttcagtggcagcgggtcagggacagatttcacactgaaaatcagccgggtggaggctgaggatgttggggtttattactgcatgcaaagtatacaacttcctcggacgttcggccaagggaccaaggtggaaatcaaa

SEQ ID NO: 204 - protein, 112 aa, Homo sapiens
DIVMTQTPLSLSVTPGQPASISCKSSQSLLHSDGKTYLYWYLQKPGQPPQLLIYEVSNRFSGVPDRFSGSGSGTDFTLKISRVEAEDVGVYYCMQSIQLPRTFGQGTKVEIK

SEQ ID NO: 205 - DNA, 360 bp, Homo sapiens
caggtgcagctacaggagtcgggcccaggactggtgaagccttcacagaccctgtccctcatctgtactgtctctggaggctccatcagccgtggtgactcctactggagctggatccgccagcacccagggaagggcctggagtggattgggtacatctattacagtgggagcaccttctacaacccgtccctcaagagtcgagttaccatatcagtagacacgtctaagaaccagttctccctgaagctgagctcagtgactgccgcggacacggccgtgtattactgtgcgagagaggataggatttttggagtggttgactactggggccagggaaccctggtcaccgtctcctca

SEQ ID NO: 206 - protein, 120 aa, Homo sapiens
QVQLQESGPGLVKPSQTLSLICTVSGGSISRGDSYWSWIRQHPGKGLEWIGYIYYSGSTFYNPSLKSRVTISVDTSKNQFSLKLSSVTAADTAVYYCAREDRIFGVVDYWGQGTLVTVSS

SEQ ID NO: 207 - DNA, 339 bp, Homo sapiens
gacatcgtgatgacccagtctccacactccctggctatgtctctgggcgagagggccaccatcaactgcaagtccagccagagtgttttatacagctccaacaataagaactacttaacttggtaccagcagaaaccaggacagcctcctaaactgctcatttactgggcatctacccgggaatccggggtccctgaccgattcagtggcagcgggtctgggacagatttcactctcaccatcagcagcctgcaggctgaagatgtggcagtttattactgtcagcaatattatagtcctccgtggacgttcggccaagggaccaaggtggaaatcaaa

SEQ ID NO: 208 - protein, 113 aa, Homo sapiens
DIVMTQSPHSLAMSLGERATINCKSSQSVLYSSNNKNYLTWYQQKPGQPPKLLIYWASTRESGVPDRFSGSGSGTDFTLTISSLQAEDVAVYYCQQYYSPPWTFGQGTKVEIK

SEQ ID NO: 209 - DNA, 374 bp, Homo sapiens
gaggtgcagctggtgcagtctggagcagaggtgaaaaagcccggggagtctctgaggatctcctgtaagggttctggatacagcgtttccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgccttcctgcagtggatcagtctgaaggcctcggacaccgccatgtattactgtgcgagactccaaggagattgtagtggtggtagctgctacgggcactttgactgctggggccagggaaccctggtcaccgtctcctc

SEQ ID NO: 210 - protein, 124 aa, Homo sapiens
EVQLVQSGAEVKKPGESLRISCKGSGYSVSSYWIGWVRQMPGKGLEWMGIIYPGDSDTRYSPSFQGQVTISADKSISTAFLQWISLKASDTAMYYCARLQGDCSGGSCYGHFDCWGQGTLVTVS

SEQ ID NO: 211 - DNA, 333 bp, Homo sapiens
gatgttgtgctgactcagtctccactctccctgcccgtcacccttggacagccggcctccatctcctgcaggtctagtcaaagcctcgtaaacagtgatggagacacctacttgagttggtttcagcagtggccaggccaatctccaaggcgcctattttatgaggtttctaactgggactctggggtcccagacagattcagcggcagtgggtcaggcactgatttcacactgaaaatcagcagggtggaggctgaggatgttggggtttattactgcatgcaaggtacacactggctcactttcggcggagggaccaaggtggagatcaaa

SEQ ID NO: 212 - protein, 111 aa, Homo sapiens
DVVLTQSPLSLPVTLGQPASISCRSSQSLVNSDGDTYLSWFQQWPGQSPRRLFYEVSNWDSGVPDRFSGSGSGTDFTLKISRVEAEDVGVYYCMQGTHWLTFGGGTKVEIK

SEQ ID NO: 213 - DNA, 360 bp, Homo sapiens
gaggtgcagctggtggagtctgggggaggcctggtcaagcctggggggtccctgagactctcctgtgcagcctctggattcaccttcagtggctatagcatgaactgggtccgccaggctccagggaaggggctggagtgggtctcatccattagtagtcgtagtagtcacatatactacgtagactcagtgaagggccgattcaccatctccagagacaacgccaagaactcactgtttctgcaaatgaacagcctgagagccgaggacacggctgtttattactgtgcgagaggattacgatattttgactggttattaaactactggggccagggaaccctggtcaccgtctcctca

SEQ ID NO: 214 - protein, 120 aa, Homo sapiens
EVQLVESGGGLVKPGGSLRLSCAASGFTFSGYSMNWVRQAPGKGLEWVSSISSRSSHIYYVDSVKGRFTISRDNAKNSLFLQMNSLRAEDTAVYYCARGLRYFDWLLNYWGQGTLVTVSS

SEQ ID NO: 215 - DNA, 321 bp, Homo sapiens
gacatccagatgacccagtctccatcctccctgtctgcatctgtaggagacagagtcaccatcacttgccaggcgagtcaggacattaacaactatttaaattggtatcagcagaaaccagggaaagcccctaagctcctgatctacgatgcatccagtttggaaacaggggtcccatcaaggttcagtggaagtggatcagggacagattttactttcaccatcagcagcctgcagcctgaagatattgcaacatattactgtcaacagtatgataatctccctcccactttcggcggagggaccaaggtggagatcaaa

SEQ ID NO: 216 - protein, 107 aa, Homo sapiens
DIQMTQSPSSLSASVGDRVTITCQASQDINNYLNWYQQKPGKAPKLLIYDASSLETGVPSRFSGSGSGTDFTFTISSLQPEDIATYYCQQYDNLPPTFGGGTKVEIK

SEQ ID NO: 217 - DNA, 354 bp, Homo sapiens
caggtgcagctggtgcagtctggggctgaggtgaggaagcctggggcctcagtgaaggtttcctgcaaggcatctggattcaccttcaccagctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagagatcccccgtccctgggcttcgatctctggggccgtggcaccctggtcactgtctcctca

SEQ ID NO: 218 - protein, 118 aa, Homo sapiens
QVQLVQSGAEVRKPGASVKVSCKASGFTFTSYYMHWVRQAPGQGLEWMGIINPSGGSTSYAQKFQGRVTMTRDTSTSTVYMELSSLRSEDTAVYYCARDPPSLGFDLWGRGTLVTVSS

SEQ ID NO: 219 - DNA, 321 bp, Homo sapiens
gaaattgtgttgacgcagtctccaggcaccctgtctttgtctccaggggaaagagccaccctctcctgcagggccagtcagagtgttagcagcaactacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtggctcattcactttcggccctgggaccaaagtggatatcaaa

SEQ ID NO: 220 - protein, 107 aa, Homo sapiens
EIVLTQSPGTLSLSPGERATLSCRASQSVSSNYLAWYQQKPGQAPRLLIYGASSRATGIPDRFSGSGSGTDFTLTISRLEPEDFAVYYCQQYGGSFTFGPGTKVDIK

SEQ ID NO: 221 - DNA, 365 bp, Homo sapiens
caggtgcagctgcaggagtcgggcccaggactggtgaagccttcggagaccctgtccctcacctgcactgtctctggtggctccatcagtttttactactggagctggatccggcagcccgccgggaagggactggagtggattgggcgtatctataccagtgggaacaccaactacaacccctccctcaagagtcgagtcaccatgtcactagacacgtccaagaaccagttctccctgaagctgagctctgtgaccgccgcggacacggccgtgtattactgtgcgagagatgatcggggagttagcgactactactacggtatggacgtctggggccaagggaccacggtcaccgtctcctc

SEQ ID NO: 222 - protein, 121 aa, Homo sapiens
QVQLQESGPGLVKPSETLSLTCTVSGGSISFYYWSWIRQPAGKGLEWIGRIYTSGNTNYNPSLKSRVTMSLDTSKNQFSLKLSSVTAADTAVYYCARDDRGVSDYYYGMDVWGQGTTVTVS

SEQ ID NO: 223 - DNA, 321 bp, Homo sapiens
gacatccagatgacccagtctccatcctccctgtctgcatctgtaggagacagagtcaccattacttgccgggcaagtcagagcattagcggctttttaaattggtttcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagttccccattcactttcggccctgggaccaaagtggatatcatt

SEQ ID NO: 224 - protein, 107 aa, Homo sapiens
DIQMTQSPSSLSASVGDRVTITCRASQSISGFLNWFQQKPGKAPKLLIYAASSLQSGVPSRFSGSGSGTDFTLTISSLQPEDFATYYCQQSYSSPFTFGPGTKVDII

SEQ ID NO: 225 - DNA, 377 bp, Homo sapiens
caggtgcagctgcaggagtcgggcccaggactggtgaagcctccggagaccctgtccctcacctgcactgtctctggcggctccatcagtagtcactactggagctggatccggcagcccgccgggaagggactggagtggattgggcgtatctataccagtgggagcaccaactacaacccccccctcaagagtcgagtcaccatgtcagtagacacgtccaagaaccagttctccctgaagctgagctctgtgaccgccgcggacacggccgtgtattactgtgcgagagaagggtacgatttttggagtggttattataactactactacggtatggacgtctggggccaagggaccacggtcaccgtctcctc

SEQ ID NO: 226 - protein, 125 aa, Homo sapiens
QVQLQESGPGLVKPPETLSLTCTVSGGSISSHYWSWIRQPAGKGLEWIGRIYTSGSTNYNPPLKSRVTMSVDTSKNQFSLKLSSVTAADTAVYYCAREGYDFWSGYYNYYYGMDVWGQGTTVTVS

SEQ ID NO: 227 - DNA, 321 bp, Homo sapiens
gaaattgtgttgacgcagtctccaggcaccctgtctttgtctccaggggaaagagccaccctctcctgcagggccagtcagagtgttatcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagcgcgttcactttcggcggagggaccaaggtggagatcaaa

SEQ ID NO: 228 - protein, 107 aa, Homo sapiens
EIVLTQSPGTLSLSPGERATLSCRASQSVISSYLAWYQQKPGQAPRLLIYGASSRATGIPDRFSGSGSGTDFTLTISRLEPEDFAVYYCQQYGSAFTFGGGTKVEIK

SEQ ID NO: 229 - DNA, 351 bp, Homo sapiens
caggtgcagctgcaggagtcgggcccaggactggtgaagccttcggagaccctgtccctcacctgcactgtctctggcggctccatcagtagttactactggagctggatccggcagcccgccgggaagggactggagtggattgggcgtatctataccagtgggagcaccaactacaacccctccctcaagagtcgagtcaccatgtcagtagacacgtccaagaaccagttctccctgaagctgagctctgtgaccgccgcggacacggccgtgtattactgtgcgggagattggggatcgagggcttttgatatctggggccaagggacaatggtcaccgtctcttca

SEQ ID NO: 230 - protein, 117 aa, Homo sapiens
QVQLQESGPGLVKPSETLSLTCTVSGGSISSYYWSWIRQPAGKGLEWIGRIYTSGSTNYNPSLKSRVTMSVDTSKNQFSLKLSSVTAADTAVYYCAGDWGSRAFDIWGQGTMVTVSS

SEQ ID NO: 231 - DNA, 315 bp, Homo sapiens
gacatccagatgacccagtctccatcttccgtgtctgcttctgtaggagacagagtcaccatcacttgtcgggcgagtcagggtattaacaactggttagcctggtatcagcagaaaccagggaaagcccctaagctcctgatctatgctgcatccagtttgcaaagtggggtcccatcaaggttcagcggcagtggatctgggacagatttcactctcgccatcagcagcctgcagcctgaagattttgcaacttactattgtcaacaggctaaaagtctcactttcggcggagggaccaaggtggagatcaaa

SEQ ID NO: 232 - protein, 105 aa, Homo sapiens
DIQMTQSPSSVSASVGDRVTITCRASQGINNWLAWYQQKPGKAPKLLIYAASSLQSGVPSRFSGSGSGTDFTLAISSLQPEDFATYYCQQAKSLTFGGGTKVEIK

SEQ ID NO: 233 - DNA, 377 bp, Homo sapiens
caggtgcagctgcaggaatcgggcccaggactggtgaagccttcggagaccctgtccctcacctgcactgtctctggtggctccatcagtagttactactggagctggatccggcagcccgccgggaagggactggagtggattgggcgtatctataccagtgggagcacccactacagcccctccctcaggagtcgagtcaccatgtcagtagacacgtccaagaaccagttctccctgaacctgagctctgtgaccgccgcggacacggccgtgtattactgtgcgagagaagggtacgatttttggagtggttattataactactactacggtatggacgtctggggccaagggaccacggtcaccgtctcctc

SEQ ID NO: 234 - protein, 125 aa, Homo sapiens
QVQLQESGPGLVKPSETLSLTCTVSGGSISSYYWSWIRQPAGKGLEWIGRIYTSGSTHYSPSLRSRVTMSVDTSKNQFSLNLSSVTAADTAVYYCAREGYDFWSGYYNYYYGMDVWGQGTTVTVS

SEQ ID NO: 235 - DNA, 321 bp, Homo sapiens
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagcgcgttcactttcggcggagggaccaaggtggagatcaaa

SEQ ID NO: 236 - protein, 107 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSAFTFGGGTKVEIK

SEQ ID NO: 237 - DNA, 365 bp, Homo sapiens
caggtgcagctgcaggagtcgggcccaggactggtgaagccttcggagaccctgtccctcacctgcactgtctctggtggctccatcagtttttactactggagctggatccggcaggccgccgggaagggactggagtggattgggcgtatccataccagtgggaacaccaacaacaacccctccctcaagagtcgagtcaccatgtcactagacacgtccaagaaccagttctccctgaagctgagctctgtgaccgccgcggacacggccgtgtattactgtgcgagagatgatcggggagttatcgactactactacggtatggacgtctggggccaagggaccacggtcaccgtctcctc

SEQ ID NO: 238 - protein, 121 aa, Homo sapiens
QVQLQESGPGLVKPSETLSLTCTVSGGSISFYYWSWIRQAAGKGLEWIGRIHTSGNTNNNPSLKSRVTMSLDTSKNQFSLKLSSVTAADTAVYYCARDDRGVIDYYYGMDVWGQGTTVTVS

SEQ ID NO: 239 - DNA, 321 bp, Homo sapiens
gacatccagatgacccagtctccatcctccctgtctgcatctgtaggagacagagtcaccattacttgccgggcaagtcagagcattagcggctttttaaattggtttcagcagaaaccagggaaagcccctaagctcctgatctatgctgcatccagtttgcaaagtggggtcccatcaaggttcagtggcagtggatctgggacagatttcactctcaccatcagcagtctgcgacctgaagattttgcaacttactcctgtcaacagagttacagtaccccattcactttcggccctgggaccaaagtggatgtcatt

SEQ ID NO: 240 - protein, 107 aa, Homo sapiens
DIQMTQSPSSLSASVGDRVTITCRASQSISGFLNWFQQKPGKAPKLLIYAASSLQSGVPSRFSGSGSGTDFTLTISSLRPEDFATYSCQQSYSTPFTFGPGTKVDVI

SEQ ID NO: 241 - DNA, 351 bp, Homo sapiens
caggtgcagctgcaggagtcgggcccaggactggtgaagccttcacagaccctgtccctcacctgcactgtctctggtggctccatcaacagtggtggttactactggagctggatccgccagcacccagggaagggcctggagtggattgggtacatcttttacagtgggagcacctactacaacccgtccctcaagagtcgagttaccatatcagtagacacgtctaagaaccagttctccctgaagctgagctctgtgactgccgcggacacggccgtgtattactgtgcgagagactcctacggtatggacgtctggggccaagggaccacggtcaccgtctcctca

SEQ ID NO: 242 - protein, 117 aa, Homo sapiens
QVQLQESGPGLVKPSQTLSLTCTVSGGSINSGGYYWSWIRQHPGKGLEWIGYIFYSGSTYYNPSLKSRVTISVDTSKNQFSLKLSSVTAADTAVYYCARDSYGMDVWGQGTTVTVSS

SEQ ID NO: 243 - DNA, 324 bp, Homo sapiens
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcttctatggtgcatccagcagggccactggcatcccagacaggttcagtggcagtgggtctgggacagacttcactctcaccatccgcagactggagcctgaagattttgccctgtattactgtcagcaatatggtcgctcacctctcactttcggcggagggaccaaggtggagatcaaa

SEQ ID NO: 244 - protein, 108 aa, Homo sapiens
EIVLTQSPGTLSLSPGERATLSCRASQSVSSSYLAWYQQKPGQAPRLLFYGASSRATGIPDRFSGSGSGTDFTLTIRRLEPEDFALYYCQQYGRSPLTFGGGTKVEIK

SEQ ID NO: 245 - DNA, 368 bp, Homo sapiens
caggtgcagctgcaggagtcgggcccaggactggtgaagccttcacagaccctgtccctcacctgcactgtctctggtggctccatcagcagtggtggttactactggagctggatccgccagcacccagggaagggcctggagtggattgggtacatctattacagtgggagcacctactacaacccgtccctcaagagtcgagttaccatatcagtagacacgtctaagaaccagttctccctgaagctgagctctgtgactgccgcggacacggccgtgtattactgtgcgagaggtctctacggtgactacggggggtactggttcgacccctggggccagggaaccctggtcaccgtctcctc

SEQ ID NO: 246 - protein, 122 aa, Homo sapiens
QVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARGLYGDYGGYWFDPWGQGTLVTVS

SEQ ID NO: 247 - DNA, 324 bp, Homo sapiens
gacatccagatgacccagtctccatcctccctgtctgcatctgtaggagacagagtcaccatcacttgccgggcaagtcagagcattagcaactatttaaattggtatcagcagaaaccagggaaagcccctaagctcctgatctatgttgcatccagtttgcaaagtggggtcccatcaaggttcagtggcagtggatctgggacagatttcactctcaccatcagcagtctgcaacctgaagattttgcaacttactactgtcaacagagttacagtaccctcatattcactttcggccctgggaccaaagtggatatcaaa

SEQ ID NO: 248 - protein, 108 aa, Homo sapiens
DIQMTQSPSSLSASVGDRVTITCRASQSISNYLNWYQQKPGKAPKLLIYVASSLQSGVPSRFSGSGSGTDFTLTISSLQPEDFATYYCQQSYSTLIFTFGPGTKVDIK

SEQ ID NO: 249 - DNA, 360 bp, Homo sapiens
caggtgcagctgcaggagtcgggcccaggactggggaagccttcacagaccctgtccctcacctgcactgtctctggtggctccatcagcagtggtggtcactactggagctggatccgccagcacccagggaagggcctggagtggattgggtacatctattacagtgggagcacctactacaacccgtccctcaagagtcgagttaccatatcagtagacacgtctaagaaccagttctccctgaagctgagctctgtgactgccgcggacacggccgtgtattactgtgcgagagatgacgtgcggtatgggggtatggacgtctggggccaagggaccacggtcaccgtctcctca

SEQ ID NO: 250 - protein, 120 aa, Homo sapiens
QVQLQESGPGLGKPSQTLSLTCTVSGGSISSGGHYWSWIRQHPGKGLEWIGYIYYSGSTYYNPSLKSRVTISVDTSKNQFSLKLSSVTAADTAVYYCARDDVRYGGMDVWGQGTTVTVSS

SEQ ID NO: 251 - DNA, 339 bp, Homo sapiens
gacatcgtgatgacccagtctccagactccctggctgtgtctctgggcgagagggccaccatcaactgcaagtccagccagagtgttgtatacagctccaacaataagaattacttagcttggtaccagcagaaaccaggacagcctcataagctgctcatttactgggcatctattcgggaatccggggtccctgaccgattcagtggcagcgggtctgggacagatttcactctcaccatcagcagcctgcaggctgaagatgtggcagtttatttctgtcagcaacattatagtactccgtggacgttcggccaagggaccaaggtggaaatcaaa

SEQ ID NO: 252 - protein, 113 aa, Homo sapiens
DIVMTQSPDSLAVSLGERATINCKSSQSVVYSSNNKNYLAWYQQKPGQPHKLLIYWASIRESGVPDRFSGSGSGTDFTLTISSLQAEDVAVYFCQQHYSTPWTFGQGTKVEIK

SEQ ID NO: 253 - DNA, 366 bp, Homo sapiens
caggtgcaactggtgcagtctggggctgaggtgaagaagcctggggcctcagtgaaggtctcctgcaaggcttctggatacaccttcaccagttatgatatcaactgggtgcgacaggccactggacaagggcttgagtggatgggatggatgaaccctaacagtggtaacacaggctatgcacagaagttccagggcagagtcaccatgaccaggaacacctccataagcacagcctacatggagctgagcagcctgcgatctgaggacacggccgtgtattactgtgcgaggaactggaacgacttctactacgacttcggtatggacgtctggggccaagggaccacggtcaccgtctcctca

SEQ ID NO: 254 - protein, 122 aa, Homo sapiens
QVQLVQSGAEVKKPGASVKVSCKASGYTFTSYDINWVRQATGQGLEWMGWMNPNSGNTGYAQKFQGRVTMTRNTSISTAYMELSSLRSEDTAVYYCARNWNDFYYDFGMDVWGQGTTVTVSS

SEQ ID NO: 255 - DNA, 339 bp, Homo sapiens
gacatcgtgatgacccagtctccagactccctggctgtgtctctgggcgagagggccaccatcaactgcaagtccagccagagtgttttatacagctccaacaataagaactacttagcttggtaccagcagaaaccaggacagcctcctaagttgctcatttactgggcatctacccgggaatccggggtccctgaccgattcagtggcagcgggtctgggacagatttcactctcaccatcagcagcctgcaggctgaagatgtggcagtttattactgtcagcaatattatagtactcctctcactttcagcggagggaccaaggtggagatcaaa

SEQ ID NO: 256 - protein, 113 aa, Homo sapiens
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPLTFSGGTKVEIK

SEQ ID NO: 257 - DNA, 360 bp, Homo sapiens
gaggtgcagctggtggagtctgggggaggcttggtaaagcctggggggtcccttagactctcctgtgcagcctctggattcactttcagtaacgcctggatgagctgggtccgccaggctccagggaaggggctggagtgggttggccgtattaaaagcaaaactgagggtgggacaacagacaatgttgcacccgtgaaaggcagattcaccatctcaagagatgattcaaaaaacacgctgtatctgcaaatgaacagcctgaaaaccgaggacacagccgtgtattattgtaccattacaacccactactacggtatggacgtctggggccaagggaccacggtcaccgtctcctca

SEQ ID NO: 258 - protein, 120 aa, Homo sapiens
EVQLVESGGGLVKPGGSLRLSCAASGFTFSNAWMSWVRQAPGKGLEWVGRIKSKTEGGTTDNVAPVKGRFTISRDDSKNTLYLQMNSLKTEDTAVYYCTITTHYYGMDVWGQGTTVTVSS

SEQ ID NO: 259 - DNA, 321 bp, Homo sapiens
gacatccagatgacccagtctccatcctccctgtctgcatctgtaggagacagagtcaccatcacttgccgggcaagtcagaccattaacaactatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactcctgtcaacagagtaacagtcccccattcactttcggccctgggaccaaagtggatgtcaaa

SEQ ID NO: 260 - protein, 107 aa, Homo sapiens
DIQMTQSPSSLSASVGDRVTITCRASQTINNYLNWYQQKPGKAPKLLIYAASSLQSGVPSRFSGSGSGTDFTLTISSLQPEDFATYSCQQSNSPPFTFGPGTKVDVK

SEQ ID NO: 261 - DNA, 360 bp, Homo sapiens
caggtgcagctgcaggagtcgggcccaggactggtgaagccttcacagaccctgtccctcacctgcactgtctctggtggctctatcagcagtggtggttactactggagctggatccgccagcacccagggaagggcctggagtggattgggttcatctatttcagtgggggcacctactacaacccgtccctcaagagtcgggttaccatatcagtggacacgtctaagaaccagttctccctgaagctgagctctgtgactgccgcggacacggccgtgtattactgtgcgagagatcgtttcgactttcgagcttttgatatctggggccaagggacaatggtcaccgtctcttca

SEQ ID NO: 262 - protein, 120 aa, Homo sapiens
QVQLQESGPGLVKPSQTLSLTCTVSGGSISSGGYYWSWIRQHPGKGLEWIGFIYFSGGTYYNPSLKSRVTISVDTSKNQFSLKLSSVTAADTAVYYCARDRFDFRAFDIWGQGTMVTVSS

SEQ ID NO: 263 - DNA, 336 bp, Homo sapiens
gatattgtgatgacccagactccactctctctgtccgtcacccctggacagccggcctccatctcctgcaagtctagtcagagcctcctacatagtgatggaaagacctatttgttttggttcctgcagaagccaggccagcctccacagctcctgatctatgaagtttccaaccggttctctggagtgccagataggttcagtggcagcgggtcagggacagatttcacactgaaaatcagccgggtggaggctgaggatgttggggtttattactgcatgcaaagtatacagcttcctcggacgttcggccaagggaccaaggtggaaatcaaa

SEQ ID NO: 264 - protein, 112 aa, Homo sapiens
DIVMTQTPLSLSVTPGQPASISCKSSQSLLHSDGKTYLFWFLQKPGQPPQLLIYEVSNRFSGVPDRFSGSGSGTDFTLKISRVEAEDVGVYYCMQSIQLPRTFGQGTKVEIK

SEQ ID NO: 265 - DNA, 383 bp, Homo sapiens
caggtgcagctggtgcagtctggggctgaggtgaagaagcctggggcctcagtgaaggtttcctgcaaggcatctggatacaccttcaccagtaactatatgcactgggtgcgacaggcccctggacaagggcttgagtggatgggaataatcaaccctagtggtggtagcacaaggtacgcacagaagtttcagggcagagtcaccatgaccagggacacgtccacgagcacagtctacatggagctgagcagcctgagatctgaggacacggccgagtattactgtgcgagagatcgaggtatagtgggagcgattatgcgggacaactactactacggtatggacgtctggggccaagggaccacggtcaccgtctcctc

SEQ ID NO: 266 - protein, 127 aa, Homo sapiens
QVQLVQSGAEVKKPGASVKVSCKASGYTFTSNYMHWVRQAPGQGLEWMGIINPSGGSTRYAQKFQGRVTMTRDTSTSTVYMELSSLRSEDTAEYYCARDRGIVGAIMRDNYYYGMDVWGQGTTVTVS

SEQ ID NO: 267 - DNA, 333 bp, Homo sapiens
gatattgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactatcaccttcggccaagggacacgactggagattaaa

SEQ ID NO: 268 - protein, 111 aa, Homo sapiens
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTITFGQGTRLEIK

SEQ ID NO: 269 - DNA, 360 bp, Homo sapiens
caggtgcacctggtgcagtctggggctgaggtgaagaagcctggggcctcagtgaaggtctcctgcaaggcttctggatacaccttcaccggctactatattcattgggtgcgacaggcccctggacaagggcttgagtggatgggatggatcaaccctaacagtggtggcacaaactatgcacagaagtttcagggcagggtcaccatgaccagggacacgtccatcagcacagcctacatggagctgagcaggctgagatctgacgacacggccgtgtattactgtgcgagggccgtattacgatattttgaccactttgactactggggccagggaaccctggtcaccgtctcctca

SEQ ID NO: 270 - protein, 120 aa, Homo sapiens
QVHLVQSGAEVKKPGASVKVSCKASGYTFTGYYIHWVRQAPGQGLEWMGWINPNSGGTNYAQKFQGRVTMTRDTSISTAYMELSRLRSDDTAVYYCARAVLRYFDHFDYWGQGTLVTVSS

SEQ ID NO: 271 - DNA, 321 bp, Homo sapiens
gacatccagatgacccagtctccatcctccctgtctgcatctgtaggagacagagtcaccatcacttgccgggcaagtcagagcattagcagctatttaaattggtatcagcagaaactagggaaagcccccaagctcctgatctatgatgtatccagtttgcaaagtggggtcccatcaaggttcagtggcagtggagctgggacagatttcactctcaccatcagcagtctgcaacctgaagattttgcaacttactactgtcaacagagtttcagtacccctccgacgttcggccaagggaccaaggtgaaaatcaaa

SEQ ID NO: 272 - protein, 107 aa, Homo sapiens
DIQMTQSPSSLSASVGDRVTITCRASQSISSYLNWYQQKLGKAPKLLIYDVSSLQSGVPSRFSGSGAGTDFTLTISSLQPEDFATYYCQQSFSTPPTFGQGTKVKIK

SEQ ID NO: 273 - DNA, 377 bp, Homo sapiens
caggtgcagctgcaggagtcgggcccaggactggtgaagccttcggagaccctgtccctcacctgcactgtctctggtggctccttcagtagttactactggagctggatccggcagcccgccgggaagggactggagtggattgggcgtatctataccagtgggagcacctactacaacccctccctcaagagtcgagtcaccatgtcagtagacacgtccaagaaccagttctccctgaagctgaactctgtgaccgccgcggacacggccgtgtattactgtgcgagagaagggtacgatttttggaatgattattataactactactacggtatggacgtctggggccaggggaccacggtcaccgtctcctc

SEQ ID NO: 274 - protein, 125 aa, Homo sapiens
QVQLQESGPGLVKPSETLSLTCTVSGGSFSSYYWSWIRQPAGKGLEWIGRIYTSGSTYYNPSLKSRVTMSVDTSKNQFSLKLNSVTAADTAVYYCAREGYDFWNDYYNYYYGMDVWGQGTTVTVS

SEQ ID NO: 275 - DNA, 321 bp, Homo sapiens
gaaattgtgttgacgcagtctccaggcaccctgtctttgtctccaggggaaagagccaccctctcctgcagggccagtcagagtgttagcagcaggtacttagcctggtaccagcagaaacctggccaggctcccaggctcctcatctatggtgcatccagcagggccactggcatcccagacaggttcagtggcagtgggtctgggacagacttcactctcaccatcagtagactggagcctgaagattttgcagtgtattactgtcagcagtatggtagcgcgttcactttcggcggagggaccaaggtggagatcaaa

SEQ ID NO: 276 - protein, 107 aa, Homo sapiens
EIVLTQSPGTLSLSPGERATLSCRASQSVSSRYLAWYQQKPGQAPRLLIYGASSRATGIPDRFSGSGSGTDFTLTISRLEPEDFAVYYCQQYGSAFTFGGGTKVEIK

SEQ ID NO: 277 - DNA, 360 bp, Homo sapiens
caggtgcagctggtggagtctgggggaggcgtggtccagcctgggaggtccctgagactctcctgtgcagcgtctggaatcaccttcagtagctatggcatgcactgggtccgccaggctccaggcaaggggctggagtgggtggcagttatgtggtatgagggaagtaataaatactatgcagactccgtgaagggccgattcaccatctccagagacaattccaagaacacgctgtatctgcaaatgaacagcctgagagccgaggacacggctgtgtattactgtgcgagaggggtattacgatattttgactggttatcctactggggccagggaaccctggtcaccgtctcctca

SEQ ID NO: 278 - protein, 120 aa, Homo sapiens
QVQLVESGGGVVQPGRSLRLSCAASGITFSSYGMHWVRQAPGKGLEWVAVMWYEGSNKYYADSVKGRFTISRDNSKNTLYLQMNSLRAEDTAVYYCARGVLRYFDWLSYWGQGTLVTVSS

SEQ ID NO: 279 - DNA, 321 bp, Homo sapiens
gaaatagtgatgacgcagtctccagccaccctgtctgtgtctccaggggaaagagccaccctctcctgcagggccagtcagagtgttagcagccacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggccgctcactttcggcggagggaccaaggtggagatcaaa

SEQ ID NO: 280 - protein, 107 aa, Homo sapiens
EIVMTQSPATLSVSPGERATLSCRASQSVSSHLAWYQQKPGQAPRLLIYGASTRATGIPARFSGSGSGTEFTLTISSLQSEDFAVYYCQQYNNWPLTFGGGTKVEIK

SEQ ID NO: 281 - DNA, 377 bp, Homo sapiens
caggtgcagctgcaggagtcgggcccaggactggtgaagccttcggagaccctgtccctcacctgcactgtctctggtggctccatcagtagtcactactggagctggatccggcagcccgccgggaagggactggagtggattgggcgtatctataccagtgggagcaccaactacaacccctccctcaagagtcgagtcaccatgtcagtagacacgtccaagaaccagttctccctgaagctgagatctgtgaccgccgcggacacggccgtgtattactgtgcgagagaggggtatagcagctcgtccggtcattacgactactactacggtatggacgtctggggccaagggaccacggtcaccgtctcctc

SEQ ID NO: 282 - protein, 125 aa, Homo sapiens
QVQLQESGPGLVKPSETLSLTCTVSGGSISSHYWSWIRQPAGKGLEWIGRIYTSGSTNYNPSLKSRVTMSVDTSKNQFSLKLRSVTAADTAVYYCAREGYSSSSGHYDYYYGMDVWGQGTTVTVS

SEQ ID NO: 283 - DNA, 321 bp, Homo sapiens
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcattcactttcggccctgggaccaaagtggatatcaaa

SEQ ID NO: 284 - protein, 107 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSFTFGPGTKVDIK

SEQ ID NO: 285 - DNA, 360 bp, Homo sapiens
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgaaggaagtaataaatactatgcagactccgtgaagggccgattcaccatctccagagacaattccaagaacacgctgtatctgcaaatgaacagcctgagagccgaggacacggctgtgtattactgtgcgagaggggtattacgatattttgactggttattatactggggccagggaaccctggtcaccgtctcctca

SEQ ID NO: 286 - protein, 120 aa, Homo sapiens
QVQLVESGGGVVQPGRSLRLSCAASGFTFSSYGMHWVRQAPGKGLEWVAVIWYEGSNKYYADSVKGRFTISRDNSKNTLYLQMNSLRAEDTAVYYCARGVLRYFDWLLYWGQGTLVTVSS

SEQ ID NO: 287 - DNA, 322 bp, Homo sapiens
gatatagtgatgacgcagcctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttatttctgtcagcagtataataactggcccatcaccttcggccaagggacacgactggagattaaac

SEQ ID NO: 288 - protein, 107 aa, Homo sapiens
DIVMTQPPATLSVSPGERATLSCRASQSVSSNLAWYQQKPGQAPRLLIYGASTRATGIPARFSGSGSGTEFTLTISSLQSEDFAVYFCQQYNNWPITFGQGTRLEIK

SEQ ID NO: 289 - DNA, 377 bp, Homo sapiens
caggtgcagctgcaggagtcgggcccaggactggtgaagccttcggagaccctgtccctcacctgcactgtctctggtggctccttcagtagttactactggagctggatccggcagcccgccgggaagggactggagtggattgggcgtatctataccagtgggagtaccaactacaacccctccctcaagagtcgagtcaccatgtcagtagacacgtccaagaaccagttctccctgaagctgagctctgtgaccgccgcggacacggccgtgtattactgtgcgagagaagggtacgatttttggagtggttattataactactactacggtatggacgtctggggccaggggaccacggtcaccgtctcctc

SEQ ID NO: 290 - protein, 125 aa, Homo sapiens
QVQLQESGPGLVKPSETLSLTCTVSGGSFSSYYWSWIRQPAGKGLEWIGRIYTSGSTNYNPSLKSRVTMSVDTSKNQFSLKLSSVTAADTAVYYCAREGYDFWSGYYNYYYGMDVWGQGTTVTVS

SEQ ID NO: 291 - DNA, 321 bp, Homo sapiens
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagtagactggagcctgaagattttgcagtgtattattgtcagcaatatggtagcgcgttcactttcggcggagggaccaaggtggagatcaaa

SEQ ID NO: 292 - protein, 107 aa, Homo sapiens; identical to SEQ ID NO: 236

SEQ ID NO: 293 - DNA, 386 bp, Homo sapiens
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgcagactccgtgaagggccgaatcaccatctccagagacaattccaagaacacgctgtatttgcaaatgaacagcctgagagccgaggacacggctgtgtattactgtgcgagaggggcgtattacgatttttggagtggttattatacggactactactacgatatggacgtctggggccaagggaccacggtcaccgtctcgtc

SEQ ID NO: 294 - protein, 128 aa, Homo sapiens
QVQLVESGGGVVQPGRSLRLSCAASGFTFSSYGMHWVRQAPGKGLEWVAVIWYDGSNKYYADSVKGRITISRDNSKNTLYLQMNSLRAEDTAVYYCARGAYYDFWSGYYTDYYYDMDVWGQGTTVTVS

SEQ ID NO: 295 - DNA, 324 bp, Homo sapiens
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcttctatggtgcatccagcagggccactggcatcccagacaggttcagtggcagtgggtctgggacagacttcactctcaccatcagcagactggagcctgaagattttgcagtgttttactgtcagcagtatggtagctcaccgctcactttcggcggagggaccaaggtggagatcaaa

SEQ ID NO: 296 - protein, 108 aa, Homo sapiens
EIVLTQSPGTLSLSPGERATLSCRASQSVSSSYLAWYQQKPGQAPRLLFYGASSRATGIPDRFSGSGSGTDFTLTISRLEPEDFAVFYCQQYGSSPLTFGGGTKVEIK

SEQ ID NO: 297 - DNA, 363 bp, Homo sapiens
caggtgcagttggtgcagtctggggctgaggtgaagaagcctggggcctcagtcaaggtctcctgtaaggcttctggatacaccttcaccggctactatttgcactgggtgcgacaggcccctggacaagggcttgagtggatgggatggatcaaccctaacagtggtggcacaaactatgcacagaagtttcagggcagggtcaccatgaccagggacacgtccatcagcacagcctacatggaactgagcaggctgagatctgacgacacggccgtgtgttattgtgtgagagcggaggaccaacagctgcttgatgcttttgatatctggggccaagggacaatggtcaccgtctcttca

SEQ ID NO: 298 - protein, 120 aa, Homo sapiens
QVQLVQSGAEVKKPGASVKVSCKASGYTFTGYYLHWVRQAPGQGLEWMGWINPNSGGTNYAQKFQGRVTMTRDTSISTAYMELSRLRSDDTAVCYCVRAEDQQLLDAFDIWGQGTMVTVS

SEQ ID NO: 299 - DNA, 325 bp, Homo sapiens
sgaaattgtgttgacgcagtctccagacaccctgtctttgtctccaggggaaagagccaccctctcctgcagggccagtcagagtgttagcagaagctacttagcctggtaccagcagaaacctggccaggctcccaggctcctcatttatggtgcatccagcagggccactggcatcccagacaggttcagtggcagtgggtctgggacagacttcactctcaccatcagcagactggagcctgaagattttgcagtgtatttctgtcagcagtatggtagctcacctctcactttcggcggagggaccaaggtggagatcaaa

SEQ ID NO: 300 - protein, 108 aa, Homo sapiens
EIVLTQSPDTLSLSPGERATLSCRASQSVSRSYLAWYQQKPGQAPRLLIYGASSRATGIPDRFSGSGSGTDFTLTISRLEPEDFAVYFCQQYGSSPLTFGGGTKVEIK

SEQ ID NO: 301 - DNA, 366 bp, Homo sapiens
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgaaggaagtaataaatactatgcagactccgtgaagggccgattcaccatctccagagacaattccaagaacacgctgtatctgcaaatgaacagcctgagagccgaggacacggctgtgtattactgtgcgagaggccccccggtattacgatattttgactggttcgactactggggccagggaaccctggtcaccgtctcctca

SEQ ID NO: 302 - protein, 122 aa, Homo sapiens
QVQLVESGGGVVQPGRSLRLSCAASGFTFSSYGMHWVRQAPGKGLEWVAVIWYEGSNKYYADSVKGRFTISRDNSKNTLYLQMNSLRAEDTAVYYCARGPPVLRYFDWFDYWGQGTLVTVSS

SEQ ID NO: 303 - DNA, 321 bp, Homo sapiens
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggcctctcactttcggcggagggaccaaggtggagatcaaa

SEQ ID NO: 304 - protein, 107 aa, Homo sapiens
EIVMTQSPATLSVSPGERATLSCRASQSVSSNLAWYQQKPGQAPRLLIYGASTRATGIPARFSGSGSGTEFTLTISSLQSEDFAVYYCQQYNNWPLTFGGGTKVEIK

SEQ ID NO: 305 - DNA, 360 bp, Homo sapiens
caggtgcagctggtgcagtctggggctgaggtgaagaagcctggggcctcagtgagggtctcctgcaaggcttctggatacaccttcaccggctactttatgcactgggtgcgacaggcccctggacaagggcttgagtggatgggatggatcaaccctaacagtgggggcacaaactatgcacagaagtttcagggcagggtcaccatgaccagggacacgtccatcagcacagcctccatggagctgagcaggctgagatctgacgacacggccgtgtattactgtgcgagagcccctctacgatattttgactggttggactactggggccagggaaccctggtcaccgtctcctca

SEQ ID NO: 306 - protein, 120 aa, Homo sapiens
QVQLVQSGAEVKKPGASVRVSCKASGYTFTGYFMHWVRQAPGQGLEWMGWINPNSGGTNYAQKFQGRVTMTRDTSISTASMELSRLRSDDTAVYYCARAPLRYFDWLDYWGQGTLVTVSS

SEQ ID NO: 307 - DNA, 339 bp, Homo sapiens
gacatcgtgatgacccagtctccagactccctggctgtgtctctgggcgagagggccaccatcaactgcaagtccagccagagtgttttatacagctccaacaataagaactgcttagcttggtaccagctgaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactccgctcactttcggcggagggaccaaggtggagatcaaa

SEQ ID NO: 308 - protein, 113 aa, Homo sapiens
DIVMTQSPDSLAVSLGERATINCKSSQSVLYSSNNKNCLAWYQLKPGQPPKLLIYWASTRESGVPDRFSGSGSGTDFTLTISSLQAEDVAVYYCQQYYSTPLTFGGGTKVEIK

SEQ ID NO: 309 - DNA, 360 bp, Homo sapiens
caggtgcagctggtggagtctgggggaggcgtggtccagcctgggaggtccctgagactctcctgtgcagcgtctggattcaccttcagtagctatggcatgtactgggtccgccaggctccaggcaaggggctggagtgggtggcagttatgtggtatgatggaagtaataaatactatgcagactccgtgaagggccgattcaccatctccagagacaattccaagaacacgctgtatctgcaaatgaacagcctgagagccgaggacacggctgtgtattactgtgcgagaggggtattacgatattttgactggttatcctactggggccagggaaccctggtcaccgtctcctca

SEQ ID NO: 310 - protein, 120 aa, Homo sapiens
QVQLVESGGGVVQPGRSLRLSCAASGFTFSSYGMYWVRQAPGKGLEWVAVMWYDGSNKYYADSVKGRFTISRDNSKNTLYLQMNSLRAEDTAVYYCARGVLRYFDWLSYWGQGTLVTVSS

SEQ ID NO: 311 - DNA, 322 bp, Homo sapiens
gaaatagtgatgacgcagtctccagccaccctgtctgtgtctccaggggaaagagccaccctctcctgcagggccagtcagagtgttagcagccacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattgctgtcagcactataataactggccgctcactttcggcggagggaccaaggtggagatcaagc

SEQ ID NO: 312 - protein, 107 aa, Homo sapiens
EIVMTQSPATLSVSPGERATLSCRASQSVSSHLAWYQQKPGQAPRLLIYGASTRATGIPARFSGSGSGTEFTLTISSLQSEDFAVYCCQHYNNWPLTFGGGTKVEIK

SEQ ID NO: 313 - DNA, 366 bp, Homo sapiens
caggtgcagctgcaggagtcgggcccaggactggtgaagccttcggagaccctgtccctcacctgcactgtctctggtggctccgtcagcagtggtggttactactggaactggatccggcagcccccagggaagggactggagtggattgggtacatctattacagtgggagcaccaactacaacccctccctcaagagtcgagtcaccatatcagcagacacgtccaagaaccagctctccctgaagctgagatctgtgaccgctgcggacacggccgtgtatttctgtgcgagagatcaatgggacttccactactacggtatggacgtctggggccaagggaccacggtcaccgtctcctca

SEQ ID NO: 314 - protein, 122 aa, Homo sapiens
QVQLQESGPGLVKPSETLSLTCTVSGGSVSSGGYYWNWIRQPPGKGLEWIGYIYYSGSTNYNPSLKSRVTISADTSKNQLSLKLRSVTAADTAVYFCARDQWDFHYYGMDVWGQGTTVTVSS

SEQ ID NO: 315 - DNA, 324 bp, Homo sapiens
gatattgtgttgacgcagtctccaggcaccctgtctttgtctccaggggaaagagccaccctctcctgcagggccagtcagagtgttagcaacaactacttagcctggtaccagcagaaacctggccaggctcccaggctcctcatctatggtgcgtccagctgggccactggcatcccagacaggttcagtggcagtgggtctgggacagacttcactctcaccatcagcagactggagcctgaagattttgcagtgtattactgtcagcagtataatagctctcctcggacgttcggccaagggaccaaggtggaaatcaaa

SEQ ID NO: 316 - protein, 108 aa, Homo sapiens
DIVLTQSPGTLSLSPGERATLSCRASQSVSNNYLAWYQQKPGQAPRLLIYGASSWATGIPDRFSGSGSGTDFTLTISRLEPEDFAVYYCQQYNSSPRTFGQGTKVEIK

SEQ ID NO: 317 - DNA, 366 bp, Homo sapiens
caggtgcagctgcaggagtcgggcccaggactggtgaagccttcggagaccctgtccctcacctgcactgtctctggtggctccatcagtttttactactggagctggatccggcagcccgccgggaagggactggagtggattgggcgtatctataccagtgggaacaccaactacaacccctccctcaagagtcgagtcaccatgtcactagacacgtccaagaaccagttctccctgaagctgagctctgtgaccgccgcggacacggccgtgtattactgtgcgagagatgatcggggaattagcgactactactacggtatggacgtctggggccaagggaccacggtcaccgtctcctca

SEQ ID NO: 318 - protein, 122 aa, Homo sapiens
QVQLQESGPGLVKPSETLSLTCTVSGGSISFYYWSWIRQPAGKGLEWIGRIYTSGNTNYNPSLKSRVTMSLDTSKNQFSLKLSSVTAADTAVYYCARDDRGISDYYYGMDVWGQGTTVTVSS

SEQ ID NO: 319 - DNA, 321 bp, Homo sapiens; identical to SEQ ID NO: 223

SEQ ID NO: 320 - protein, 107 aa, Homo sapiens; identical to SEQ ID NO: 224

SEQ ID NO: 321 - DNA, 377 bp, Homo sapiens
caggtgcagctgcaggagtcgggcccaggactggtgaagccttcggagaccctgtccctcacctgcactgtctctggtggctccttcagtagttactactggagctggatccggcagcccgccgggaagggactggagtggattgggcgtatctataccagtgggagcaccaactacaacccctccctcaagagtcgagtcaccatgtcagtagacacgtccaagaaccagttctccctgaagctgagctctgtgaccgccgcggacacggccgtgtattactgtgcgagagaagggtacgatttttggagtggttattataactactactacggtatggacgtctggggccaggggaccacggtcaccgtctcctc

SEQ ID NO: 322 - protein, 125 aa, Homo sapiens; identical to SEQ ID NO: 290

SEQ ID NO: 323 - DNA, 321 bp, Homo sapiens
gaagttgtgttgacgcagtctccaggcaccctgtctttgtctccaggggaaagagccaccctctcctgcagggccagtcagagtgttagcagcagctacttagcctggtaccagcagaaacctggccaggctccccggctcctcatctatggtgcatccagcagggccactggcatcccagacaggttcagtggcagtgggtctgggacagacttcactctcaccatcagtagactggagcctgaagattttgcagtgtattactgtcagcagtatggtagcgcgttcactttcggcggagggaccaaggtggagatcaaa

SEQ ID NO: 324 - protein, 107 aa, Homo sapiens
EVVLTQSPGTLSLSPGERATLSCRASQSVSSSYLAWYQQKPGQAPRLLIYGASSRATGIPDRFSGSGSGTDFTLTISRLEPEDFAVYYCQQYGSAFTFGGGTKVEIK

SEQ ID NO: 325 - DNA, 360 bp, Homo sapiens
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggcagggtcaccatgaccagggacacgtccatcagcacagcctacatggagctgagcaggctgagatctgacgacacggccgtgtattactgtgcgagaggggtattacgatattttgactggtttgactactggggccagggaaccctggtcaccgtctcctca

SEQ ID NO: 326 - protein, 120 aa, Homo sapiens
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARGVLRYFDWFDYWGQGTLVTVSS

SEQ ID NO: 327 - DNA, 339 bp, Homo sapiens
gacatcgtgatgacccagtctccagactccctggctgtgtctctgggcgagagggccaccatctactgcaagtccagccagagtgttttcgacagttccaacaataagaactccttagcttggtacctgcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatattactccgtgcagttttggccaggggacccacctggagatcaaa

SEQ ID NO: 328 - protein, 113 aa, Homo sapiens
DIVMTQSPDSLAVSLGERATIYCKSSQSVFDSSNNKNSLAWYLQKPGQPPKLLIYWASTRESGVPDRFSGSGSGTDFTLTISSLQAEDVAVYYCQQYYITPCSFGQGTHLEIK

SEQ ID NO: 329 - DNA, 351 bp, Homo sapiens
caggtgcagctgcaggagtcgggcccaggactggtgaagccttcacagaccctgtccctcacctgcactgtctctggtggctccatcagcagtggtaattactactggagctggatccgccagcacccagggaagggcctggagtggattgggtacatctattatagtgggagcacctactacaacccgtccctcaagagtcgagttatcatatcagtagacacgtctaagaaccagttctccctgaagctgagctctgtgactgccgcggacacggccgtgtattactgtgcgagagacggggacggcatggacgtctggggccaagggaccacggtcaccgtctcctca

SEQ ID NO: 330 - protein, 117 aa, Homo sapiens
QVQLQESGPGLVKPSQTLSLTCTVSGGSISSGNYYWSWIRQHPGKGLEWIGYIYYSGSTYYNPSLKSRVIISVDTSKNQFSLKLSSVTAADTAVYYCARDGDGMDVWGQGTTVTVSS

SEQ ID NO: 331 - DNA, 324 bp, Homo sapiens
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtttggtagctcaccgctcactttcggcggagggaccaaggttgagatcaaa

SEQ ID NO: 332 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQFGSSPLTFGGGTKVEIK

SEQ ID NO: 333 - DNA, 377 bp, Homo sapiens
caggtgcagctgcaggagtcgggcccaggactggtgaagccttcggagaccctgtccctcacctgcactgtctctggtggctccatcagtagtcactactggagctggatccggcagcccgccgggaagggactggagtggattgggcgtgtctattccagtgggagcaccaactccaacccctccctcaagagtcgagtcaccatgtcagtagacacggccaagaaccagttctccctgaagctgagctctgtgaccgccgcggacacggccgtgtattactgtgcgagagaagggtacgatttttggagtgattattataactactactacggtatggacgtctggggccaagggaccacggtcaccgtctcctc

SEQ ID NO: 334 - protein, 125 aa, Homo sapiens
QVQLQESGPGLVKPSETLSLTCTVSGGSISSHYWSWIRQPAGKGLEWIGRVYSSGSTNSNPSLKSRVTMSVDTAKNQFSLKLSSVTAADTAVYYCAREGYDFWSDYYNYYYGMDVWGQGTTVTVS

SEQ ID NO: 335 - DNA, 321 bp, Homo sapiens; identical to SEQ ID NO: 235

SEQ ID NO: 336 - protein, 107 aa, Homo sapiens; identical to SEQ ID NO: 236

SEQ ID NO: 337 - DNA, 360 bp, Homo sapiens
caggtgcagctggtgcagtctggggctgaggtgaagaagcctggggcctcagtgaaggtctcctgcaaggcttctggatacaccttcaccggctactttatgcactgggtgcgacaggcccctggacaagggcttgagtggatgggatggatcaaccctaacagtggtggcacaaactatgcacagaagtttcagggcagggtcaccatgaccagggacacgtccatcagcacagcctacatggagctgagcaggctgagatctgacgacacggccgtgtattactgtgcgagaagcgtattacgatattttgactggttattatggtggggccagggaaccctggtcaccgtctcctca

SEQ ID NO: 338 - protein, 120 aa, Homo sapiens
QVQLVQSGAEVKKPGASVKVSCKASGYTFTGYFMHWVRQAPGQGLEWMGWINPNSGGTNYAQKFQGRVTMTRDTSISTAYMELSRLRSDDTAVYYCARSVLRYFDWLLWWGQGTLVTVSS

SEQ ID NO: 339 - DNA, 339 bp, Homo sapiens
gacatcgtgatgacccagtctccagagtccctggctgtgtctctgggcgagaggaccaccatcaactgcaagtccagccagagtgtcttattcagctccaacaatgagaaccacttagcttggtaccagcagaaaccaggacagcctcctaagttactcatttactgggcatctacccgggaatccggggtccctgaccgattcagtggcagcgggtctgggacagatttcactctcaccatcagcagcctgcaggctgaagatgtggcagtttattactgtcagcaatattatattactccgtgcagttttggccaggggaccaagctggagatcaaa

SEQ ID NO: 340 - protein, 113 aa, Homo sapiens
DIVMTQSPESLAVSLGERTTINCKSSQSVLFSSNNENHLAWYQQKPGQPPKLLIYWASTRESGVPDRFSGSGSGTDFTLTISSLQAEDVAVYYCQQYYITPCSFGQGTKLEIK

SEQ ID NO: 341 - DNA, 389 bp, Homo sapiens
caggtgcagctgcaggagtcgggcccaggactggtgaagccttcggagaccctgtccctcacctgcactgtctctggtggctccatcagtagttactactggagctggctccggcagcccgccgggaagggactggagtggattgggcgtctctataccagtgggagcaccaactacaacccctccctcaagagtcgagtcaccatgtcagtagacacgtccaagaaccagttctccctgaagctgagctctgtaaccgccgcggacacggccgtgtattactgtgcgagagaggggtattactatgatagtagtggttattactacgattactactactactacggtatggacgtctggggccaagggaccacggtcaccgtctcctc

SEQ ID NO: 342 - protein, 129 aa, Homo sapiens
QVQLQESGPGLVKPSETLSLTCTVSGGSISSYYWSWLRQPAGKGLEWIGRLYTSGSTNYNPSLKSRVTMSVDTSKNQFSLKLSSVTAADTAVYYCAREGYYYDSSGYYYDYYYYYGMDVWGQGTTVTVS

SEQ ID NO: 343 - DNA, 324 bp, Homo sapiens
gaaattgtgttgacgcagtctccaggcaccctgtctttgtctccaggggaaagagccaccctctcctgcagggccagtcagagtgttagcagcaggtacttagcctggtaccagcagaaacctggccaggctcccaggctcctcatctatggtgcatccagcagggccactggcatcccagacagattcagtggcagtgggtctgggacagacttcactctcaccatcagcagactggagcctgaagattttgcagtgtattactgtcagcagtatggtagctcaattttcactttcggccctgggaccaaagtggatatcaaa

SEQ ID NO: 344 - protein, 108 aa, Homo sapiens
EIVLTQSPGTLSLSPGERATLSCRASQSVSSRYLAWYQQKPGQAPRLLIYGASSRATGIPDRFSGSGSGTDFTLTISRLEPEDFAVYYCQQYGSSIFTFGPGTKVDIK

SEQ ID NO: 345 - DNA, 371 bp, Homo sapiens
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggctgtgtattactgtctgagagatcgggcgtatagtgggagctactacgagggctggttcgacccctggggccagggaaccctggtcaccgtctcctc

SEQ ID NO: 346 - protein, 123 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCLRDRAYSGSYYEGWFDPWGQGTLVTVS

SEQ ID NO: 347 - DNA, 321 bp, Homo sapiens
gacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactcttacaatcagcagcctgcagcctgaagattttgcaacttattactgtctacagcataataattacccgtggacgttcggccaagggaccaaggtggaaatcaaa

SEQ ID NO: 348 - protein, 107 aa, Homo sapiens
DIQMTQSPSSLSASVGDRVTITCRASQGIRNDLGWYQQKPGKAPKRLIYAASSLQSGVPSRFSGSGSGTEFTLTISSLQPEDFATYYCLQHNNYPWTFGQGTKVEIK

SEQ ID NO: 349 - DNA, 365 bp, Homo sapiens
gaggtgcagctggtggagtctgggggaggcttggtaaagccagggcggtccctgagactctcctgtacagcttctggaatcacctttggtgattatgctttgagctggttccgccaggctccagggaaggggctggagtgggtaggtttcattagaagcgaagcttatggtgggacaacagaatacgccgcgtctgtgaaaggcagattcaccatctcaagagatgattccaaaagcatcgcctatctgcaaatgaacagcctgaaaaccgaggacacagccgtgtattactgtactagaggtggatacagctatggttacgactttgactactggggccagggaaccctggtcaccgtctcctc

SEQ ID NO: 350 - protein, 121 aa, Homo sapiens
EVQLVESGGGLVKPGRSLRLSCTASGITFGDYALSWFRQAPGKGLEWVGFIRSEAYGGTTEYAASVKGRFTISRDDSKSIAYLQMNSLKTEDTAVYYCTRGGYSYGYDFDYWGQGTLVTVS

SEQ ID NO: 351 - DNA, 336 bp, Homo sapiens; identical to SEQ ID NO: 123

SEQ ID NO: 352 - protein, 112 aa, Homo sapiens; identical to SEQ ID NO: 124

SEQ ID NO: 353 - DNA, 360 bp, Homo sapiens
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaagaaatactatgcagactccgtgaagggccgattcaccatctccagagacaattccaagaacacgctgtatctgcaaatgaacagcctgagagccgaggacacggctgtgtattactgtgcgagagattggggacacagctatggccactttgactactggggccagggaaccctggtcaccgtctcctca

SEQ ID NO: 354 - protein, 120 aa, Homo sapiens
QVQLVESGGGVVQPGRSLRLSCAASGFTFSSYGMHWVRQAPGKGLEWVAVIWYDGSKKYYADSVKGRFTISRDNSKNTLYLQMNSLRAEDTAVYYCARDWGHSYGHFDYWGQGTLVTVSS

SEQ ID NO: 355 - DNA, 336 bp, Homo sapiens
gatgttgtgatgactcagtctccactctccctgcccgtcacccttggacagccggcctccatctcctgcaggtctagtcaaagcctcgtatacagtgatggaaacacctacttgaattggtttcagcagaggccaggccaatctccaaggcgcctaatttataaggtttctaactgggactctggggtcccagacagattcagcggcagtgggtcaggcactgatttcacactgaaaatcagcagggtggaggctgaggatgttggggtttattactgcatgcaaggtacacactggcctccgacgttcggccaagggaccaaggtggagatctac

SEQ ID NO: 356 - protein, 112 aa, Homo sapiens
DVVMTQSPLSLPVTLGQPASISCRSSQSLVYSDGNTYLNWFQQRPGQSPRRLIYKVSNWDSGVPDRFSGSGSGTDFTLKISRVEAEDVGVYYCMQGTHWPPTFGQGTKVEIY

SEQ ID NO: 357 - DNA, 374 bp, Homo sapiens
caggtgcagctggtggagtctgggggaggcgtggtccagcctgggaggtccgtgagactctcctgtgcagcgtctggattcaccttcagtagctctggcatgcactgggtccgccaggctccaggcaaggggctggaatgggtggcagttatttggtatgatggaagtaataaatactatgcagactccgtgaagggccgattcaccatctccagagacaattccaagaacacgctgtatctgcaaatgaacagcctgagagccgaggacacggctgtgtattactgtgcgagagatggattctctatagcagtggccttctactactacggtatggacgtctggggccaagggaccacggtcaccgtttcctc

SEQ ID NO: 358 - protein, 124 aa, Homo sapiens
QVQLVESGGGVVQPGRSVRLSCAASGFTFSSSGMHWVRQAPGKGLEWVAVIWYDGSNKYYADSVKGRFTISRDNSKNTLYLQMNSLRAEDTAVYYCARDGFSIAVAFYYYGMDVWGQGTTVTVS

SEQ ID NO: 359 - DNA, 321 bp, Homo sapiens
gacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatacttacccaataattttcggccctgggaccaaagtggatatcaaa

SEQ ID NO: 360 - protein, 107 aa, Homo sapiens
DIQMTQSPSSLSASVGDRVTITCRASQGIRNDLGWYQQKPGKAPKRLIYAASSLQSGVPSRFSGSGSGTEFTLTISSLQPEDFATYYCLQHNTYPIIFGPGTKVDIK

SEQ ID NO: 361 - DNA, 374 bp, Homo sapiens
caagtgcagctggtggagtctgggggaggcgtggtccagcctgggaggtccctgagactctcctgtgcagcgtctggattcaccttcagtacccatggcatgcactgggtccgccaggctccaggcaaggggctggagtgggtggcagttatatggtatgttggtagtaataaataccatgcagactccgtgaagggccgattcaccatctccagagacaattccaagaacacgctgtatctgcaaatgaacagcctgagagccgaggacacggctgtgtattactgtgcgagagatagtttccgaatggtaactggatcccactactacggtatggacgtctggggccaagggaccacggtcaccgtctcctc

SEQ ID NO: 362 - protein, 124 aa, Homo sapiens
QVQLVESGGGVVQPGRSLRLSCAASGFTFSTHGMHWVRQAPGKGLEWVAVIWYVGSNKYHADSVKGRFTISRDNSKNTLYLQMNSLRAEDTAVYYCARDSFRMVTGSHYYGMDVWGQGTTVTVS

SEQ ID NO: 363 - DNA, 321 bp, Homo sapiens
gacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacaacataatatttaccctccgacgttcggccaagggaccaaggtggaaatcaaa

SEQ ID NO: 364 - protein, 107 aa, Homo sapiens
DIQMTQSPSSLSASVGDRVTITCRASQGIRNDLGWYQQKPGKAPKRLIYAASSLQSGVPSRFSGSGSGTEFTLTISSLQPEDFATYYCLQHNIYPPTFGQGTKVEIK

SEQ ID NO: 365 - DNA, 372 bp, Homo sapiens
caggtgcagctgcagcagtcgggcccaggactggtgaagccttcggagaccctgtccctcacctgcactgtctctggtgcctccatcagtagttactactggagttggatccggcagcccgccgggaagggactggagtggattggtcgtatctatgccagtgggagtaccaactacaacccctccctcaagagtcgagtcaccatgtcagtagacacgtccaagagccagttctccctgaacctgagctctgtgaccgccgcggacacggccgtgtattactgtgcgagagaggggtgggagcgactacaggactactactactacggtttggacgtctggggccaagggaccacggtcaccgtctcctca

SEQ ID NO: 366 - protein, 124 aa, Homo sapiens
QVQLQQSGPGLVKPSETLSLTCTVSGASISSYYWSWIRQPAGKGLEWIGRIYASGSTNYNPSLKSRVTMSVDTSKSQFSLNLSSVTAADTAVYYCAREGWERLQDYYYYGLDVWGQGTTVTVSS

SEQ ID NO: 367 - DNA, 324 bp, Homo sapiens
gaaattgtgttgacgcagtctccaggcaccctgtctttgtctccaggggaaagagccaccctctcctgcagggccagtcagagtgttagcaggaactacttagcctggaaccaacagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcaatatggtagctcaccgctcactttcggcggagggaccaaggtggagatcaaa

SEQ ID NO: 368 - protein, 108 aa, Homo sapiens
EIVLTQSPGTLSLSPGERATLSCRASQSVSRNYLAWNQQKPGQAPRLLIYGASSRATGIPDRFSGSGSGTDFTLTISRLEPEDFAVYYCQQYGSSPLTFGGGTKVEIK

SEQ ID NO: 369 - DNA, 351 bp, Homo sapiens
caggtgcagctgcaggagaggggcccaggactggtgaagccttcggagaccctgtccctcacctgcactgtctctggtggctccatcagtaggtactactggagctggatccggcagcccgccgggaagggactggagtggattgggcgtatctataccagtgggagcaccaactacaacccctccctcaagagtcgagtcaccatgtcagtagacacgtccaagaaccagttctccctgaagctgagctctgtgaccgccgcggacacggccgtgtattactgtgcgagaggttggctggtggaatacttccagcactggggccagggcaccctggtcaccgtctcctca

SEQ ID NO: 370 - protein, 117 aa, Homo sapiens
QVQLQERGPGLVKPSETLSLTCTVSGGSISRYYWSWIRQPAGKGLEWIGRIYTSGSTNYNPSLKSRVTMSVDTSKNQFSLKLSSVTAADTAVYYCARGWLVEYFQHWGQGTLVTVSS

SEQ ID NO: 371 - DNA, 321 bp, Homo sapiens
gacatccagatgacccagtctccatcctccctgtctgcatctgtaggagacagagtcaccatcacttgccaggcgagtcaggacattagcatctatttaaattggtatcagcagaaaccagggaaagcccctaagctcctgatcttcgatgtatccaatttggaaacaggggtcccatcaaggttcagtggaagtggatctgggacagattttactttcaccatcagcagcctgcagcctgaagatattgcaacatattactgtcaacagtatgataatctcccgctcactttcggcggagggaccaaggtggagatcaaa

SEQ ID NO: 372 - protein, 107 aa, Homo sapiens
DIQMTQSPSSLSASVGDRVTITCQASQDISIYLNWYQQKPGKAPKLLIFDVSNLETGVPSRFSGSGSGTDFTFTISSLQPEDIATYYCQQYDNLPLTFGGGTKVEIK

SEQ ID NO: 373 - DNA, 371 bp, Homo sapiens
caggtgcagctggtggagtctgggggaggcgtggtccagcctgggaggtccctgagactctcctgtgaagcgtctggattcaccttcagtagctatggcatgcactgggtccgccaggctccaggcaaggggctggagtgggtggcagttatatggtatgctggaagtaataaatactgtgcagactccgtgaagggccgattcaccatctccagagacaattccaagaacacgctgtatctgcaaatgaacagcctgagagccgaggacacggctgtgtattactgtgcgagagaggcctttactatggggaggtaccactactacggtatggacgtctggggccaagggaccacggtcaccgtctcctc

SEQ ID NO: 374 - protein, 123 aa, Homo sapiens
QVQLVESGGGVVQPGRSLRLSCEASGFTFSSYGMHWVRQAPGKGLEWVAVIWYAGSNKYCADSVKGRFTISRDNSKNTLYLQMNSLRAEDTAVYYCAREAFTMGRYHYYGMDVWGQGTTVTVS

SEQ ID NO: 375 - DNA, 321 bp, Homo sapiens
gacatccagatgacccagtctccatcctccctgtctgcatctgtaggagacagagtcaccatcacttgccgggcaagtcagggcattagaaatgatttaggctggtatcagcagaaaccagggaaagcccctaaacgactgatctttgctgcatccagtttgcaaagtggggtcccatcaaggttcagcggcagtggatctgggacagaattcactctcacaatcagcagcctgcagcctgaagattttgcaacttattactgtctacagcataatagttacccgtgcagttttggccaggggaccaaggtggagatcaaa

SEQ ID NO: 376 - protein, 107 aa, Homo sapiens
DIQMTQSPSSLSASVGDRVTITCRASQGIRNDLGWYQQKPGKAPKRLIFAASSLQSGVPSRFSGSGSGTEFTLTISSLQPEDFATYYCLQHNSYPCSFGQGTKVEIK

SEQ ID NO: 377 - DNA, 357 bp, Homo sapiens
caggttcagctggtgcagtctggagctgaggtgaagaagcctggggcctcagtgaaggtctcctgcaaggcttctggtttcacctttaccagttatggtatcagctgggtgcgacaggcccctggacaagggcttgagtggatgggatggatcaacgcttccacaggtaaaacaaactttgcacagaagctccagggcagagtcaccatgaccacagacacatccacgaacacagcctacatggagttgaggagcctgagatctgacgacacggccgtgtattactgtgcgcgagagctgggtctggcagcccctatggacgtctggggccaagggaccacggtcaccgtctcctca

SEQ ID NO: 378 - protein, 119 aa, Homo sapiens
QVQLVQSGAEVKKPGASVKVSCKASGFTFTSYGISWVRQAPGQGLEWMGWINASTGKTNFAQKLQGRVTMTTDTSTNTAYMELRSLRSDDTAVYYCARELGLAAPMDVWGQGTTVTVSS

SEQ ID NO: 379 - DNA, 321 bp, Homo sapiens
gacatccagatgacccagtctccatcctccctgtctgcatctgtgggagacagagtcaccatcacttgccgggcaagtcagaacattaacagctttttaaattggtatcagcagaaaccagggaaagcccctaagctcctgatctatgctgcatccagtttgcaaagtggggtcccatcagggttcagtggcagtggatctgggacaggtttcactctcaccatcagcagtctgcaacctgaagattttgcaacttactactgtcaacagagttacagtaccccattcactttcggccctgggaccaaagtggatatcgag

SEQ ID NO: 380 - protein, 107 aa, Homo sapiens
DIQMTQSPSSLSASVGDRVTITCRASQNINSFLNWYQQKPGKAPKLLIYAASSLQSGVPSGFSGSGSGTGFTLTISSLQPEDFATYYCQQSYSTPFTFGPGTKVDIE

SEQ ID NO: 381 - DNA, 377 bp, Homo sapiens
gaggtgcagctggaggagtctgggggaggcctggtcaagcctggggggtccttgagactctcctgtgcagcctctggattcaccttcagtagctataccatgaactgggtccgccaggctccagggaaggggctggagtgggtctcatcccttagtagtagtagtacttacatatactacgcagactcagtgaagggccgattcaccatctccagagacaacgccaagaactcactgtatctgcacatgaacagcctgagagccgaggacacggctgtgtattactgtgcgagagatgaagcagcagctggtcccgattacttctactactacggtatggacgtctggggccaagggaccacggtcaccgtctcctc

SEQ ID NO: 382 - protein, 125 aa, Homo sapiens
EVQLEESGGGLVKPGGSLRLSCAASGFTFSSYTMNWVRQAPGKGLEWVSSLSSSSTYIYYADSVKGRFTISRDNAKNSLYLHMNSLRAEDTAVYYCARDEAAAGPDYFYYYGMDVWGQGTTVTVS

SEQ ID NO: 383 - DNA, 321 bp, Homo sapiens
gacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactcttacaatcagcagcctgcagcctgaagattttgcaacttattactgtctacagcataatagttacccgtgcagttttggccaggggaccaagctggagatcaaa

SEQ ID NO: 384 - protein, 107 aa, Homo sapiens
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPCSFGQGTKLEIK

SEQ ID NO: 385 - DNA, 355 bp, Homo sapiens
caggtgcagctggtgcagtctggggctgaggtgaagaagcctggggcctcagtgaaggtctcctgcaaggcttctggatacaccttcaccggctactatatgcactgggcgcgacaggcccctggacaagggcttgagtggatgggctggatcaaccctaacagtggtggcgcaaactatgaacagaagtttcagggcagggtcaccatgaccagggacacgtccatcagcacagcctacatggagctgagcaggctgagatctgacgacacggccgtgtattactgtgcgagagataggcagtgggggtacttcgatctctggggccgtggcaccctggtcactgtctcctcag

SEQ ID NO: 386 - protein, 118 aa, Homo sapiens
QVQLVQSGAEVKKPGASVKVSCKASGYTFTGYYMHWARQAPGQGLEWMGWINPNSGGANYEQKFQGRVTMTRDTSISTAYMELSRLRSDDTAVYYCARDRQWGYFDLWGRGTLVTVSS

SEQ ID NO: 387 - DNA, 321 bp, Homo sapiens
gacatccagatgacccagtctccatcctccctgtctgcatctgtaggagacagagtcaccatcacttgccgggcaagtcagagcattagcggctgtttaaattggtatcagcagaaaccagggaaagcccctaagctcctgatctatgaaacatccagtttgcaaagtggggtcccatcaaggttcagtggcagtggatctgggacagatctcactctcaccatcagcagtctacaacctgaagattttgcaacatactactgtcaacagagttacagtaccccgctcactttcggcggagggaccaaggtggagatcaaa

SEQ ID NO: 388 - protein, 107 aa, Homo sapiens
DIQMTQSPSSLSASVGDRVTITCRASQSISGCLNWYQQKPGKAPKLLIYETSSLQSGVPSRFSGSGSGTDLTLTISSLQPEDFATYYCQQSYSTPLTFGGGTKVEIK

SEQ ID NO: 389 - DNA, 378 bp, Homo sapiens
gaagtgcagctggtggagtctgggggagtcgtggtacagcctggggggtccctgagactctcctgtgcagcctctggtttcacctttgatgattataccatgcactgggtccgtcaagctccggggaagggtctggagtgggtctctcttattatttgggatggtggtagcacatactatgcagactctgtgaagggccgattcaccatctccagagacaacagcaaaaactccctgtatctgcaaatgaacagtctgagaactgaggacaccgccttgtattactgtgcaaaagatacccgatacagctatggtccttactactactactacggtatggacgtctggggccaagggaccacggtcaccgtctcctca

SEQ ID NO: 390 - protein, 126 aa, Homo sapiens
EVQLVESGGVVVQPGGSLRLSCAASGFTFDDYTMHWVRQAPGKGLEWVSLIIWDGGSTYYADSVKGRFTISRDNSKNSLYLQMNSLRTEDTALYYCAKDTRYSYGPYYYYYGMDVWGQGTTVTVSS

SEQ ID NO: 391 - DNA, 336 bp, Homo sapiens
gatattgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactccggtcactttcggcggagggaccaaggtggagatcaaa

SEQ ID NO: 392 - protein, 112 aa, Homo sapiens
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPVTFGGGTKVEIK

SEQ ID NO: 393 - DNA, 360 bp, Homo sapiens
caggtgcagctggtggagtctgggggaggcgtggtccagcctgggaggtccctgagactctcctgtgcagcgtctggaatcaccttcagtagctatggcatgcactgggtccgccaggctccaggcaaggggctggagtgggtggcagttatatggtatgatggaagcaataaacactatgcagactccgtgaagggccgattcaccatctccagagacaattccaagaacacgctgtttctgcaaatgaacagcctgagagccgaggacacggctgtgtattactgtgcgggggggataactggacctacggaggactttgactactggggccagggaaccctggtcaccgtctcctca

SEQ ID NO: 394 - protein, 120 aa, Homo sapiens
QVQLVESGGGVVQPGRSLRLSCAASGITFSSYGMHWVRQAPGKGLEWVAVIWYDGSNKHYADSVKGRFTISRDNSKNTLFLQMNSLRAEDTAVYYCAGGITGPTEDFDYWGQGTLVTVSS

SEQ ID NO: 395 - DNA, 337 bp, Homo sapiens
gatgttgtgatgactcagtctccaatttcattgtccgtcacccttggacagccggcctccatctcctgcaggtctagtcaaagcctcgtatacagtgatggaaacacctacttgaattggtttcagcagaggccaggccaatctccaaggcgcctaatttataaggtttctaactgggactctggggtcccagacagaatcagcggcagtgggtcaggcactgatttcacactgaaaatcagcagggtggaggctgaggatgttggggtctattactgcatgcaaggtacatactggcctccaagttttggccaggggaccaagctggagatcaaac

SEQ ID NO: 396 - protein, 112 aa, Homo sapiens
DVVMTQSPISLSVTLGQPASISCRSSQSLVYSDGNTYLNWFQQRPGQSPRRLIYKVSNWDSGVPDRISGSGSGTDFTLKISRVEAEDVGVYYCMQGTYWPPSFGQGTKLEIK

SEQ ID NO: 397 - DNA, 387 bp, Homo sapiens
caggtgcagctggtggagtctgggggaggcgtggtccagcctgggaggtccctgagactctcctgtgtagcgtctggattcaccttcagtagctatggcatgcactgggtccgccaggctccaggcaaggggctggagtgggtggcacttatatggtatgatggaagtaataaatactatgcagactccgtgaagggccgattcaccatctccagagacatttccaagaacacgctgtatctgcaaatgaacagcctgagagccgaggacacggctgtgtattactgtgcgagagatcccattactatggttcggggagtcccatattactactactacttcggtatggacgtctggggccaagggaccacggtcaccgtctcctca

SEQ ID NO: 398 - protein, 129 aa, Homo sapiens
QVQLVESGGGVVQPGRSLRLSCVASGFTFSSYGMHWVRQAPGKGLEWVALIWYDGSNKYYADSVKGRFTISRDISKNTLYLQMNSLRAEDTAVYYCARDPITMVRGVPYYYYYFGMDVWGQGTTVTVSS

SEQ ID NO: 399 - DNA, 327 bp, Homo sapiens
gacatccagatgacccagtctccatcctccctgtctgcatctgtaggagacagagtcaccatcacttgccggacaagtcagagcattagctgctgtgtaaattggtatcagcagaaagcagggaaagcccctaaactcctgatctttgctacatccaggttgcaaagtggggtcccaacaaggttcagtggcagtggatccgggacagatttcactctcaccatcagcagtctgcaacctgaagattttgcaacttactactgtcaacagagttacagtgcccctccggagctcactttcggcggagggaccaaggtggagatcaaa

SEQ ID NO: 400 - protein, 109 aa, Homo sapiens
DIQMTQSPSSLSASVGDRVTITCRTSQSISCCVNWYQQKAGKAPKLLIFATSRLQSGVPTRFSGSGSGTDFTLTISSLQPEDFATYYCQQSYSAPPELTFGGGTKVEIK

SEQ ID NO: 401 - DNA, 346 bp, Homo sapiens
gaggtgcagctgttggagtctgggggaggcttggtacagcctggggggtccctgagactctcctgtgcagcctctggattcacctttagcacctatggcatgagctgggtccgccaggctccagggaaggggctggagggggtctcagttattagtggtagtggtggtagcacatactacgcagactccgtgaagggccggttcaccatctccagagacaattccaagaacacgctgtatctgcaaatgaacagcctgagagtcgaggacacggccgtatattactgtgcgaaaggggccctctttgacaactggggccagggaaccctggtcaccgtctcctcag

SEQ ID NO: 402 - protein, 115 aa, Homo sapiens
EVQLLESGGGLVQPGGSLRLSCAASGFTFSTYGMSWVRQAPGKGLEGVSVISGSGGSTYYADSVKGRFTISRDNSKNTLYLQMNSLRVEDTAVYYCAKGALFDNWGQGTLVTVSS

SEQ ID NO: 403 - DNA, 321 bp, Homo sapiens
gacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttacaaagtggggtcccatcaaggttcagcggcagtggatctgggacagaattcactctcacaatcagcagcctgcagcctgaagattttgcaacttattactgtctacagcataatagttacccattcactttcggccctgggaccaaagtggatatcaaa

SEQ ID NO: 404 - protein, 107 aa, Homo sapiens
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPFTFGPGTKVDIK

SEQ ID NO: 405 - DNA, 357 bp, Homo sapiens
caggttcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtcacacaaactatgcacagaagctccagggcagagtcaccatgaccacagacacatccacgagcacagcctacatggagctgaggagcctgagatctgacgacacggccgtgtattactgtgcgagagattggggcagcctcggagcttttggtatctggggccaagggacaatggtcaccgtctcttca

SEQ ID NO: 406 - protein, 119 aa, Homo sapiens
QVQLVQSGAEVKKPGASVKVSCKASGYTFTSYGISWVRQAPGQGLEWMGWISAYNGHTNYAQKLQGRVTMTTDTSTSTAYMELRSLRSDDTAVYYCARDWGSLGAFGIWGQGTMVTVSS

SEQ ID NO: 407 - DNA, 318 bp, Homo sapiens
gacatccagatgacccagtctccatcctccctgtctgcatctgtaggggacagagtcaccatcacttgccgggcaagtcagagcattcgcagttatttaaattggtttcagcagaaaccagggaaagcccctaagctcctgatctctgctgcatccagtttgcaaagtggggtcccatcaagattcagtggcagtggatctgggacagatttcactctcaccatcagcagtctgcaacctgaagattttgcaacttactactgtcaacagagttacagtaccatcaccttcggccaagggacacgactggagattaaa

SEQ ID NO: 408 - protein, 106 aa, Homo sapiens
DIQMTQSPSSLSASVGDRVTITCRASQSIRSYLNWFQQKPGKAPKLLISAASSLQSGVPSRFSGSGSGTDFTLTISSLQPEDFATYYCQQSYSTITFGQGTRLEIK

SEQ ID NO: 409 - DNA, 360 bp, Homo sapiens
gaggtgcagctggtggagtctgggggaggcctggtcaagcctggggggtccctgagactctcctgtgcagcctctggattcatcttcagtagctatagcatgaactgggtccgccaggctccggggaaggggctggagtgggtctcatccattagtagcagtagtagttacatacactatgcagactcagtgaagggccgattcaccatctccagagacaacgccaagaactcactgtatctgcaaatgaacagcctgagagccgaggacacggctgtgtattactgtgcgagagataagatagcagtggctggcctttttgactactggggccagggaaccctggtcaccgtctcctca

SEQ ID NO: 410 - protein, 120 aa, Homo sapiens
EVQLVESGGGLVKPGGSLRLSCAASGFIFSSYSMNWVRQAPGKGLEWVSSISSSSSYIHYADSVKGRFTISRDNAKNSLYLQMNSLRAEDTAVYYCARDKIAVAGLFDYWGQGTLVTVSS

SEQ ID NO: 411 - DNA, 339 bp, Homo sapiens
gacatcgtgatgacccagtctccagactccctggctgtgtctctgggcgagagggccaccatcaactgcaagtccagccagagtgttttatacagctccaacaataagaactacttaacttggtaccaacagaaaccaggacagcctcctaaactactcatttactgggcatctacccgggaatccggggtccctgcccgattcagtggcagcgggtctgggacagatttcactctcaccatcagcggcctgcaggctgaagatgtggcagtttattactgtcagcaatattatagtattccgtggacgttcggccaagggaccaaggtggaaatcaaa

SEQ ID NO: 412 - protein, 113 aa, Homo sapiens
DIVMTQSPDSLAVSLGERATINCKSSQSVLYSSNNKNYLTWYQQKPGQPPKLLIYWASTRESGVPARFSGSGSGTDFTLTISGLQAEDVAVYYCQQYYSIPWTFGQGTKVEIK

SEQ ID NO: 413 - protein, 1179 aa, Homo sapiens
MAPRPRARPGVAVACCWLLTVVLRCCVSFNVDVKNSMTFSGPVEDMFGYTVQQYENEEGKWVLIGSPLVGQPKNRTGDVYKCPVGRGESLPCVKLDLPVNTSIPNVTEVKENMTFGSTLVTNPNGGFLACGPLYAYRCGHLHYTTGICSDVSPTFQVVNSIAPVQECSTQLDIVIVLDGSNSIYPWDSVTAFLNDLLERMDIGPKQTQVGIVQYGENVTHEFNLNKYSSTEEVLVAAKKIVQRGGRQTMTALGIDTARKEAFTEARGARRGVKKVMVIVTDGESHDNHRLKKVIQDCEDENIQRFSIAILGSYNRGNLSTEKFVEEIKSIASEPTEKHFFNVSDELALVTIVKTLGERIFALEATADQSAASFEMEMSQTGFSAHYSQDWVMLGAVGAYDWNGTVVMQKASQIIIPRNTTFNVESTKKNEPLASYLGYTVNSATASSGDVLYIAGQPRYNHTGQVIIYRMEDGNIKILQTLSGEQIGSYFGSILTTTDIDKDSNTDILLVGAPMYMGTEKEEQGKVYVYALNQTRFEYQMSLEPIKQTCCSSRQHNSCTTENKNEPCGARFGTAIAAVKDLNLDGFNDIVIGAPLEDDHGGAVYIYHGSGKTIRKEYAQRIPSGGDGKTLKFFGQSIHGEMDLNGDGLTDVTIGGLGGAALFWSRDVAVVKVTMNFEPNKVNIQKKNCHMEGKETVCINATVCFDVKLKSKEDTIYEADLQYRVTLDSLRQISRSFFSGTQERKVQRNITVRKSECTKHSFYMLDKHDFQDSVRITLDFNLTDPENGPVLDDSLPNSVHEYIPFAKDCGNKEKCISDLSLHVATTEKDLLIVRSQNDKFNVSLTVKNTKDSAYNTRTIVHYSPNLVFSGIEAIQKDSCESNHNITCKVGYPFLRRGEMVTFKILFQFNTSYLMENVTIYLSATSDSEEPPETLSDNVVNISIPVKYEVGLQFYSSASEYHISIAANETVPEVINSTEDIGNEINIFYLIRKSGSFPMPELKLSISFPNMTSNGYPVLYPTGLSSSENANCRPHIFEDPFSINSGKKMTTSTDHLKRGTILDCNTCKFATITCNLTSSDISQVNVSLILWKPTFIKSYFSSLNLTIRGELRSENASLVLSSSNQKRELAIQISKDGLPGRVPLWVILLSAFAGLLLLMLLILALWKIGFFKRPLKKKMEK

SEQ ID NO: 414 - protein, 214 aa, Homo sapiens
VSPTFQVVNSIAPVQECSTQLDIVIVLDGSNSIYPWDSVTAFLNDLLERMDIGPKQTQVGIVQYGENVTHEFNLNKYSSTEEVLVAAKKIVQRGGRQTMTALGIDTARKEAFTEARGARRGVKKVMVIVTDGESHDNHRLKKVIQDCEDENIQRFSIAILGSYNRGNLSTEKFVEEIKSIASEPTEKHFFNVSDELALVTIVKTLGERIFALEA

SEQ ID NO: 415 - protein, 214 aa, Mus musculus
VSPTFQVVNSFAPVQECSTQLDIVIVLDGSNSIYPWESVTAFLNDLLKRMDIGPKQTQVGIVQYGANVTHEFNLNKYSSTEEVLVAANKIGRRGGLQTMTALGIDTARKEAFTEARGARRGVKKVMVIVTDGESHDNYRLKQVIQDCEDENIQRFSIAILGHYNRGNLSTEKFVEEIKSIASEPTEKHFFNVSDELALVTIVKALGERIFALEA

SEQ ID NO: 416 - protein, 214 aa, Rattus norvegicus
VSPTFQVVNSFAPVQECSTQLDIVIVLDGSNSIYPWESVIAFLNDLLKRMDIGPKQTQVGIVQYGENVTHEFNLNKYSSTEEVLVAANKIGRQGGLQTMTALGIDTARKEAFTEARGARRGVKKVMVIVTDGESHDNYRLKQVIQDCEDENIQRFSIAILGHYNRGNLSTEKFVEEIKSIASEPTEKHFFNVSDELALVTIVKALGERIFALEA

SEQ ID NO: 417 - protein, 7 aa, Homo sapiens
VQRGGRQ

SEQ ID NO: 418 - protein, 6 aa, Mus musculus
GRRGGL

SEQ ID NO: 419 - protein, 6 aa, Homo sapiens
VQRGGR

(19) United States
(12) Patent Application Publication
(10) Pub. No.: US 2006/0286112 A1
(43) Pub. Date: Dec. 21, 2006

(54) Human monoclonal antibodies that bind to very late antigen-1 for the treatment of inflammation and other disorders

(76) Inventors: Sirid-Aimee Kellermann, Menlo Park, CA (US); Orit Foord, Foster City, CA (US); Shelley Sims Belouski, Camarillo, CA (US); Larry L. Green, San Francisco, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/433,924

(22) Filed: May 12, 2006

Related U.S. Application Data
(60) Provisional application No. 60/681,846, filed on May 16, 2005.

(51) Int. Cl. G01N 33/574; C07H 21/04; C12P 21/06; A61K 39/395; C07K 16/30; C12N 5/06

(52) U.S. Cl. 424/155.1; 530/388.8; 530/388.15; 435/69.1; 435/320.1; 435/328; 536/23.53; 435/7.23

(57) ABSTRACT

The present invention relates to antibodies directed to very late antigen-1 (“VLA-1”) and uses of such antibodies. For example human monoclonal antibodies directed to VLA-1 are described. Isolated polynucleotide sequences encoding, and amino acid sequences comprising, heavy and light chain immunoglobulin molecules, particularly sequences corresponding to contiguous heavy and light chain sequences spanning the framework regions (FR's) and/or complementarity determining regions (CDR's), are provided. Hybridomas or other cell lines expressing such immunoglobulin molecules and monoclonal antibodies are also provided.

### REFERENCE TO RELATED APPLICATIONS

The present application claims the priority benefit under 35 U.S.C. §119(e) of U.S. Provisional Application No: 60/681,846, filed May 16, 2005, the entirety of which is incorporated herein by reference.

### FIELD OF INVENTION

The present invention relates generally to antibodies to Very Late Antigens. More particularly, the present invention relates to fully human antibodies to VLA-1.

### BACKGROUND OF THE INVENTION

Members of a protein family called very late antigens (VLA) are expressed on the surface of white blood cells and other cell types. The VLA proteins constitute a family of six distinct heterodimers (VLA-1-VLA-6) that all share a common 110 kDa (non-reduced, Mᵣ) beta subunit but differ in their alpha subunits, which range in their molecular weights from 200 to 130 kDa (alpha1-alpha6) (Hemler, M. E. et al., J. Biol. Chem., 262(7), 3300, (1987); Hemler, M. E. et al., J. Biol. Chem., 263, 7660, (1988)). The amino terminus of each subunit or chain forms a globular head that contributes to intersubunit linking and to ligand binding. The globular heads are connected to the transmembrane segments by stalks. The cytoplasmic tails are usually less than 50 amino acid residues long. For a general review, see Cellular and Molecular Immunology (eds. Abul K. Abbas et al., W. B. Saunders Company, Philadelphia, Pa., 2000).

Collagen (both types I and IV) and laminin are known ligands of VLA-1 (i.e., alpha1/beta1 integrin). VLA-1 proteins are known to provide anchorage as well as signals for cellular growth, migration, and differentiation during development and tissue repair. They have been implicated in immune and inflammatory processes. VLA-1 has been implicated in cell adhesion and migration on collagen (Keely et al., 1995, J. Cell Sci. 108:595-607; and Gotwals et al., 1996, J. Clin. Invest. 97:2469-2477); in promoting contraction and reorganization of collagen matrices, a critical component of wound healing (Gotwals et al., supra; and Chiro, 1991, Cell 67:403-410); and in regulating the expression of genes involved in extracellular matrix remodeling (Riikonen et al., 1995, J. Biol. Chem. 270: 1-5; and Langholz et al., 1995, J. Cell Biol. 131:1903-1915).

### SUMMARY OF THE INVENTION

Embodiments of the invention relate to isolated antibodies, or fragments thereof, that specifically bind to VLA.

In some aspects, the present invention relates to a human antibody or antigen-binding portion thereof that binds to very late antigen-1 (“VLA-1”). The isolated fully human antibody binds to the alpha subunit of very late antigen-1 with a K_{D} of no more than about 200 pM and the antibody inhibits the interaction between very late antigen-1 and a molecule selected from the group consisting of collagen IV, collagen I, and laminin.

In some aspects, the present invention relates to a human antibody or antigen-binding portion thereof that binds to an alpha subunit of very late antigen-1 (“VLA-1”). The human antibody, or antigen-binding portion thereof, has an IC₅₀ of no more than about 5 nM for preventing VLA-1 mediated adhesion to collagen IV.

In some aspects, the present invention relates to a human antibody or antigen-binding portion thereof that binds to an alpha subunit of very late antigen-1 (“VLA-1”). The human antibody, or antigen-binding portion thereof, has an IC₅₀ of no more than 5 nM for inhibiting an interaction between immobilized collagen IV and an I domain of very late antigen-1.

In some aspects, the present invention relates to a method of treating a subject suffering from a very late antigen-1 (“VLA-1”) mediated disorder. The method comprises selecting a subject in need of treatment for a very late antigen-1 mediated disorder and administering to the subject a therapeutically effective dose of a human monoclonal antibody or antigen-binding portion thereof that specifically binds to very late antigen-1. The antibody has a KD of no more than 200 pM, and inhibits the interaction between very late antigen-1 and collagen IV.

In some aspects, the present invention relates to the use of a human antibody or antigen-binding portion thereof of any of the antibodies described herein in the preparation of a medicament for the treatment of a very late antigen-1 (“VLA-1”) mediated disorder.

In some aspects, the present invention relates to a kit for treating very late antigen-1 related disorders. The kit comprises a human very late antigen-1 antibody, or antigen-binding portion thereof in a unit dose and instructions for administering the unit dose of the very late antigen-1 antibody to a subject.

In some aspects, the present invention relates to a kit for detecting very late antigen-1 in a cell or to screen for very late antigen-1 related disorders. The kit comprises a human antibody, or antigen-binding portion thereof, that binds to very late antigen-1 with a KD of no more than about 200 pM and a means for indicating a binding of the antibody with very late antigen-1.

In some aspects, the present invention relates to an isolated nucleic acid sequence encoding the antibody, or antigen-binding portion thereof of any of the disclosed antibodies. In some aspects, the present invention relates to a host cell transformed with the nucleic acid sequence of any of the antibodies.

In some aspects, the present invention relates to a method of diagnosing a very late antigen-1 (“VLA-1”) mediated disorder comprising using the human very late antigen-1 antibody, or antigen-binding portion thereof, to detect the level of very late antigen-1 in a patient sample. The amount of the very late antigen-1 in the sample is higher than a predetermined amount, indicating that the patient is suffering from a very late antigen-1 mediated disorder.

In some aspects, the present invention relates to a human antibody or antigen-binding portion thereof that binds to very late antigen-1 (“VLA-1”). The human antibody or antigen-binding portion thereof binds to an epitope comprising amino acid position E66 on CD49a.

In some aspects, the present invention relates to a human antibody or antigen-binding portion thereof that binds to very late antigen-1 (“VLA-1”), wherein the human antibody or antigen-binding portion thereof binds to an epitope comprising amino acid position K142 on CD49a.

In some aspects, the present invention relates to a human antibody or antigen-binding portion thereof that binds to very late antigen-1 (“VLA-1”), wherein the human antibody or antigen-binding portion thereof binds to an epitope comprising amino acid position S162 on CD49a. In some aspects, the present invention relates to a human antibody or antigen-binding portion thereof that binds to VLA-1 and that cross-competes with antibody 1.7 when binding to VLA-1. In some aspects, the present invention relates to a human antibody or antigen-binding portion thereof that binds to VLA-1 and that cross-competes with antibody 2.7 when binding to VLA-1. In some aspects, the present invention relates to a human antibody or antigen-binding portion thereof that binds to VLA-1 and that cross-competes with an antibody in bin 3.

In some aspects, the present invention relates to a composition comprising the human antibody or antigen-binding portion thereof of any of the antibodies described herein and a pharmaceutically acceptable carrier.

In some aspects, an isolated fully human antibody that binds to VLA-1 is provided. The isolated fully human antibody binds to the alpha subunit of VLA-1 with a KD of no more than about 200 pM, and the antibody inhibits the interaction between VLA-1 and collagen IV, VLA-1 and collagen I, or VLA-1 and laminin. In one embodiment the K_{D} is no more than about 100 pM, 50 pM, or 25 pM. In another embodiment, the antibody binds to a region of VLA-1 that prevents VLA-1 from signaling through an interaction with a VLA-1 ligand. In one embodiment the KD values are determined in the presence of no Mg²⁺ or in low amounts of Mg²⁺.

In another aspect, an isolated fully human antibody that binds to the alpha subunit of VLA-1 is provided. The isolated fully human antibody has an IC₅₀ of no more than about 5 or 10 nM for preventing T cell adhesion in vitro, and the antibody inhibits the interaction between VLA-1 and collagen IV, VLA-1 and collagen I, and/or VLA-1 and laminin by binding to the alpha subunit of VLA-1. In another embodiment, the antibody has an IC₅₀ of no more than about 10 nM, 1000 pM, 500 pM, 100 pM, or 10 pM in preventing T cell adhesion. In another embodiment, the isolated fully human antibody has an IC₅₀ of no more than about 100 pM in a CHO-alpha 1 potency assay.

In some embodiments, the above antibody or antigen-binding portion thereof is a monoclonal antibody. In some embodiments, a composition comprising the monoclonal antibody or antigen-binding portion of one of the above antibodies and a pharmaceutically acceptable carrier are provided.

In another aspect, a kit for treating VLA-1 related disorders is provided. The kit comprises a VLA-1 antibody in a unit dose, and instructions for administering the VLA-1 antibody to a subject. In another embodiment, the antibody does not bind to mouse VLA-1. In another embodiment, the antibody binds to native cynomolgus (Macaca fascicularis) VLA-1.

In some embodiments, the antibody inhibits the binding of proteins containing the VLA-1 alpha subunit (CD49a) to collagen. In some embodiments, the CD49a is further transfected into CHO cells and the antibody prevents the binding of the transfected CHO cells to a collagen IV matrix. In another embodiment, the antibody inhibits the collagen IV-mediated transmigration of activated T cells and the inhibition occurs in vitro. In another embodiment, the antibody further inhibits the effector phase of delayed type of hypersensitivity in vivo. In another embodiment, the antibody inhibits the induction of arthritis.

In another embodiment, the antibody comprises a heavy chain amino acid sequence comprising a complementarity determining region (CDR) of a CDR of SEQ ID NO.: 2, 6, 10, 14, 18, 22, 26, 30, 34, 38, 42, 46, 50, 54, 58, 62, 66, 70, 74, 78, 82, 86, 90, 94, 98, 102, 106, 110, 114, 118, 122, 126, 130, 134, 138, 142, 146, 150, 154, 158, 162, 166, 170, 174, 178, 182, 186, 190, 194, 198, 202, 206, 210, 214, 218, 222, 226, 230, 234, 238, 242, 246, 250, 254, 258, 262, 266, 270, 274, 278, 282, 286, 290, 294, 298, 302, 306, 310, 314, 318, 322, 326, 330, 334, 338, 342, 346, 350, 354, 358, 362, 366, 370, 374, 378, 382, 386, 390, 394, 398, 402, 406, or 410. In another embodiment, the antibody that binds to VLA-1 comprises a light chain amino acid sequence having a CDR of one of the CDR sequences of SEQ ID NO: 4, 8, 12, 16, 20, 24, 28, 32, 36, 40, 44, 48, 52, 56, 60, 64, 68, 72, 76, 80, 84, 88, 92, 96, 100, 104, 108, 112, 116, 120, 124, 128, 132, 136, 140, 144, 148, 152, 156, 160, 164, 168, 172, 176, 180, 184, 188, 192, 196, 200, 204, 208, 212, 216, 220, 224, 228, 232, 236, 240, 244, 248, 252, 256, 260, 264, 268, 272, 276, 280, 284, 288, 292, 296, 300, 304, 308, 312, 316, 320, 324, 328, 332, 336, 340, 344, 348, 352, 356, 360, 364, 368, 372, 376, 380, 384, 388, 392, 396, 400, 404, 408, or 412.

In another embodiment, the antibody specifically binds to VLA-1 and inhibits binding of VLA-1 to collagen, thereby inhibiting VLA-1-mediated disorders. In another embodiment, the antibody comprises a heavy chain complementarity determining region 1 (CDR1) with an amino acid sequence of a CDR1 in SEQ ID NO.: 2, 6, 10, 14, 18, 22, 26, 30, 34, 38, 42, 46, 50, 54, 58, 62, 66, 70, 74, 78, 82, 86, 90, 94, 98, 102, 106, 110, 114, 118, 122, 126, 130, 134, 138, 142, 146, 150, 154, 158, 162, 166, 170, 174, 178, 182, 186, 190, 194, 198, 202, 206, 210, 214, 218, 222, 226, 230, 234, 238, 242, 246, 250, 254, 258, 262, 266, 270, 274, 278, 282, 286, 290, 294, 298, 302, 306, 310, 314, 318, 322, 326, 330, 334, 338, 342, 346, 350, 354, 358, 362, 366, 370, 374, 378, 382, 386, 390, 394, 398, 402, 406, or 410; a heavy chain complementarity determining region 2 (CDR2) with an amino acid sequence of a CDR2 in SEQ ID NO: 2, 6, 10, 14, 18, 22, 26, 30, 34, 38, 42, 46, 50, 54, 58, 62, 66, 70, 74, 78, 82, 86, 90, 94, 98, 102, 106, 110, 114, 118, 122, 126, 130, 134, 138, 142, 146, 150, 154, 158, 162, 166, 170, 174, 178, 182, 186, 190, 194, 198, 202, 206, 210, 214, 218, 222, 226, 230, 234, 238, 242, 246, 250, 254, 258, 262, 266, 270, 274, 278, 282, 286, 290, 294, 298, 302, 306, 310, 314, 318, 322, 326, 330, 334, 338, 342, 346, 350, 354, 358, 362, 366, 370, 374, 378, 382, 386, 390, 394, 398, 402, 406, or 410; and a heavy chain complementarity determining region 3 (CDR3) with an amino acid sequence of a CDR3 in SEQ ID NO: 2, 6, 10, 14, 18, 22, 26, 30, 34, 38, 42, 46, 50, 54, 58, 62, 66, 70, 74, 78, 82, 86, 90, 94, 98, 102, 106, 110, 114, 118, 122, 126, 130, 134, 138, 142, 146, 150, 154, 158, 162, 166, 170, 174, 178, 182, 186, 190, 194, 198, 202, 206, 210, 214, 218, 222, 226, 230, 234, 238, 242, 246, 250, 254, 258, 262, 266, 270, 274, 278, 282, 286, 290, 294, 298, 302, 306, 310, 314, 318, 322, 326, 330, 334, 338, 342, 346, 350, 354, 358, 362, 366, 370, 374, 378,382, 386,390, 394, 398, 402, 406, or 410.

In another embodiment, the antibody comprises a heavy chain amino acid comprising the amino acid sequence shown in SEQ ID NO: 2 or 54. In another embodiment, the antibody comprises a light chain amino acid comprising the amino acid sequence shown in SEQ ID NO: 4 or 56. In another embodiment, antibody binding to CD49a is dependent on residue K142 on CD49a. In another embodiment, the antibody binding to CD49a is dependent on residue E66, S162, or both on CD49a. In another embodiment, the antibody binding to CD49a is dependent on any of the residues identified herein, including E48, V91, Q92, and R96. In another embodiment, the antibody comprises a light chain complementarity determining region 1 (CDR1) having an amino acid sequence of a CDR1 in SEQ ID NO.: 4, 8, 12, 16, 20, 24, 28, 32, 36, 40, 44, 48, 52, 56, 60, 64, 68, 72, 76, 80, 84, 88, 92, 96, 100, 104, 108, 112, 116, 120, 124, 128, 132, 136, 140, 144, 148, 152, 156, 160, 164, 168, 172, 176, 180, 184, 188, 192, 196, 200, 204, 208, 212, 216, 220, 224, 228, 232, 236, 240, 244, 248, 252, 256, 260, 264, 268, 272, 276, 280, 284, 288, 292, 296, 300, 304, 308, 312, 316, 320, 324, 328, 332, 336, 340, 344, 348, 352, 356, 360, 364, 368, 372, 376, 380, 384, 388, 392, 396, 400, 404, 408, or 412; a light chain complementarity determining region 2 (CDR2) having an amino acid sequence of a CDR2 in SEQ ID NO.: 4, 8, 12, 16, 20, 24, 28, 32, 36, 40, 44, 48, 52, 56, 60, 64, 68, 72, 76, 80, 84, 88, 92, 96, 100, 104, 108, 112, 116, 120, 124, 128, 132, 136, 140, 144, 148, 152, 156, 160, 164, 168, 172, 176, 180, 184, 188, 192, 196, 200, 204, 208, 212, 216, 220, 224, 228, 232, 236, 240, 244, 248, 252, 256, 260, 264, 268,;272, 276, 280, 284, 288, 292, 296, 300, 304, 308, 312, 316, 320, 324, 328, 332, 336, 340, 344, 348, 352, 356, 360, 364, 368, 372, 376, 380, 384, 388, 392, 396, 400, 404, 408, or 412; and a light chain complementarity determining region 3 (CDR3) having an amino acid sequence of a CDR3 in SEQ ID NO: 4, 8, 12, 16, 20, 24, 28, 32, 36, 40, 44, 48, 52, 56, 60, 64, 68, 72, 76, 80, 84, 88, 92, 96, 100, 104, 108, 112, 116, 120, 124, 128, 132, 136, 140, 144, 148, 152, 156, 160, 164, 168, 172, 176, 180, 184, 188, 192, 196, 200, 204, 208, 212, 216, 220, 224, 228, 232, 236, 240, 244, 248, 25.2, 256, 260, 264, 268, 272, 276, 280, 284, 288, 292, 296, 300, 304, 308, 312, 316, 320, 324, 328, 332, 336, 340, 344, 348, 352, 356, 360, 364, 368, 372, 376, 380, 384, 388, 392, 396, 400, 404, 408, or 412.

In another embodiment, the antibody comprises a heavy chain complementarity determining region 1 (CDR1) corresponding to canonical class 1; a heavy chain complementarity determining region 2 (CDR2) corresponding to canonical class 4; a light chain complementarity determining region 1 (CDR1) corresponding to canonical class 6; a light chain complementarity determining region 2 (CDR2) corresponding to canonical class 1; and a light chain complementarity determining region 3 (CDR3) corresponding to canonical class 5.

In another aspect, a method for assaying the level of VLA-1 in a patient sample is provided. The method comprises contacting a VLA-1 antibody from one of the claimed antibodies above with a biological sample from a patient and detecting the level of binding between the antibody and VLA-1 in the sample. In one embodiment, the biological sample is blood.

In one aspect, a composition comprising a fully human VLA-1 antibody or functional fragment thereof described above and a pharmaceutically acceptable carrier is provided.

In another aspect, a method of effectively treating an animal suffering from an VLA-1 mediated disorder is provided. The method comprises selecting an animal in need of treatment for a treatable VLA-1 mediated disorder and administering to the animal a therapeutically effective dose of a fully human monoclonal antibody that specifically binds to VLA-1, wherein the antibody has a KD of no more than 200 pM, and the antibody inhibits the interaction between VLA-1 and a) collagen IV, b) collagen I, and/or c) laminin.

In another aspect, an isolated nucleic acid molecules encoding any one of the antibodies described above is provided. In another aspect, a host cell transformed with a nucleic acid molecule for any of the above antibodies is provided.

In another embodiment, the antibody will bind to human VLA-1 but will not effectively bind to murine VLA-1. In another embodiment, the antibody will bind to both human and cynomolgus VLA-1.

In another aspect, a method of diagnosing a VLA-1 mediated disorder is provided. The method comprises using the VLA-1 antibody of claim 1 to detect the level of VLA-1 in a patient sample. In one embodiment, the patient sample is blood or blood serum. In another embodiment, the VLA-1 mediated disorder is selected from the group consisting of immune-mediated inflammatory disorders (IMIDs), which are inflammatory conditions caused and sustained by an antigen-specific, pathological immune response. Among these disorders /are various types of arthritis, such as rheumatoid arthritis, as well as allergic diseases, such as asthma, hay fever, and urticaria; different types of connective tissue disorders; inflammatory bowel disease (e.g., Crohn's disease and ulcerative colitis); insulin-dependent diabetes; uveitis; retinitis; graft rejection; and graft-versus host-disease. VLA-1 mediated disorders can also include tissue inflammation in infectious, ischemic, hemorrhagic, and traumatic conditions, e.g., fasciitis, stroke, infarction of the myocardium and other organs (e.g., lung and intestine), ARDS; hepatitis, (e.g., infectious and non-infectious, acute and chronic); acute and chronic pancreatitis; reperfusion injuries; radiation injuries; vascular restenosis of different types (e.g., coronary restenosis). Also included are osteoarthritis, osteoporosis, atherosclerosis, organ fibrosis, and neurodegenerative disorders such as Alzheimer's disease and multiple sclerosis. Disorders in which VLA-1 blockade may have therapeutic benefit can include cancer, blood malignancies, e.g., leukemias and multiple myelomas; the development of a number of solid tumors, tumor growth, and metastatic spreading. As will be appreciated by one of skill in the art, in some embodiments, the antibodies disclosed herein can be used to not only identify the above disorders, but to also treat, cure, or prevent such disorders. As such, methods and compositions for the detection, treatment, prevention, etc. of such disorders involving the herein disclosed antibodies are contemplated for the above disorders and related disorders. The above list can also serve as examples of treatable VLA-1 related disorders.

In another aspect, an assay kit for detecting VLA-1 in mammalian tissues or cells to screen for VLA-1 related disorders is provided. The kit comprises an antibody that binds to a VLA-1 with a KD of no more than about 200 pM, and the antibody inhibits the interaction between VLA-1 and collagen IV. The kit further comprises a means for indicating a reaction of the antibody with the VLA-1. In another embodiment, the antibody is a monoclonal antibody. In another embodiment, the antibody that binds VLA-1 is labeled. In another embodiment, the antibody is an unlabeled primary antibody and the kit further includes a means for detecting the primary antibody. In another embodiment, the means includes a labeled second antibody that is an anti-immunoglobulin. In another embodiment, the antibody is labeled with a marker selected from the group consisting of a fluorochrome, an enzyme, a radionuclide and a radiopaque material.

In another aspect, a method for treating diseases or conditions associated with the expression of VLA-1 in a patient is provided. The method comprises administering to the patient an effective amount of a fully human VLA-1 antibody. The antibody binds to a VLA-1 protein with a KD of no more than about 200 pM, and the antibody inhibits the interaction between VLA-1 and collagen IV, collagen I, and/or laminin. In another embodiment, the antibody is administered in combination with an additional antibody. In another embodiment, the additional antibody is selected from the group consisting of an oligoclonal and polyclonal mixture of VLA-1 antibodies. In another embodiment, the method is performed in vivo and the patient is preferably a human patient. In another embodiment, the disease or condition is selected from the group consisting of immune-mediated inflammatory disorders (IMIDs) such as rheumatoid arthritis and psoriasis; asthma, hay fever, and urticaria; connective tissue disorders; inflammatory bowel disease (e.g., Crohn's disease and ulcerative colitis); insulin-dependent diabetes; uveitis; retinitis; graft rejection; graft-versus host-disease; fasciitis; stroke, infarction of the myocardium and other organs (e.g., lung and intestine), ARDS; hepatitis, (e.g., infectious and non-infectious, acute and chronic); acute and chronic pancreatitis; reperfusion injuries; radiation injuries; vascular restenosis of different types (e.g., coronary restenosis); osteoarthritis; osteoporosis; atherosclerosis; organ fibrosis; neurodegenerative disorders such as Alzheimer's disease and multiple sclerosis, and blood and solid cancers.

In another aspect, a composition for treating VLA-1 mediated disorders is provided. The composition comprises a container, the container comprises a unit dose of human anti-VLA-1 antibody. The antibody binds to a VLA-1 protein with a KD of no more than about 200 pM, and the antibody inhibits the interaction between VLA-1 and collagen IV, collagen I, and/or laminin. The kit also comprises a package insert which indicates that the composition can be used to treat VLA-1 mediated disorders characterized by the overexpression of VLA-1. In another embodiment, the antibody described above hinders VLA-1 mediated adhesion of phorbol myristate acetate activated T cells to collagen with a KD of not more than 300 pM or an IC₅₀ of no more than about 600, 200, or 100 pM.

Further embodiments include an isolated antibody, or fragment thereof, that comprises a heavy chain amino acid sequence. Other embodiments include an isolated antibody, or fragment thereof, that comprises a heavy chain nucleic acid sequence.

It will be appreciated that in these embodiments, the isolated antibodies can be monoclonal antibodies, chimeric antibodies and/or human or humanized antibodies. Preferably, the antibodies are human antibodies.

It will also be appreciated that embodiments of the invention are not limited to any particular form of an antibody. For example, the antibodies provided may be a full length antibody (e.g. having an intact human Fc region) or an antibody fragment (e.g. a Fab, Fab′ or F(ab′)₂). In addition, the antibodies may be manufactured from a hybridoma that secretes the antibody, or from a recombinantly produced cell that has been transformed or transfected with a gene or genes encoding the antibody.

Other embodiments include isolated nucleic acid molecules encoding any of the antibodies described herein.

In yet further embodiments, the invention provides an isolated polynucleotide molecule described herein.

### BRIEF DESCRIPTION OF THE DRAWINGS

The invention will be better understood from the Detailed Description and from the appended drawings, which are meant to illustrate and not to limit the invention.

FIG. 1 depicts the sequence of the human alpha 1 subunit of VLA-1. The first blocked section of text is the signal peptide, the second is the I domain, and the third blocked section has two parts, the transmembrane (TM) and the cytoplasmic section.

FIG. 2 is an alignment between amino acid sequences of the I domain of the alpha 1 subunit of VLA-1 from various organisms to show the conserved and most likely functional areas of this part of the protein.

FIG. 3 is a bar graph depicting the relative potencies of the different antibodies at three different concentrations, 10, 0.5, and 0.1 μg/ml, in an cellular adhesion assay.

FIGS. 4A-4H depicts a series of alignments showing a comparison of the heavy chains of various VLA-1 antibodies.

FIGS. 4I-4P depicts a series of alignments showing a comparison of the light chains of various VLA-1 antibodies.

FIG. 5 is a depiction of the structure of the CD49a I domain, and the residues that are involved in forming epitopes for some of the antibodies to VLA-1.

FIG. 6A is a bar graph showing antibodies (10 micrograms/ml final concentration) that were tested for their ability to inhibit adhesion of IL-2 activated human peripheral blood T cells to Collagen IV. White bars, no PMA. Black bars, adhesion in the presence of 10 ng/ml PMA.

FIGS. 6B and 6C are bar graphs showing antibodies that were tested at 5 nM (black bars) or 500 pM (grey bars) in an adhesion assay, in the absence (6B) and presence (6C) of 10 ng/ml PMA.

### DETAILED DESCRIPTION

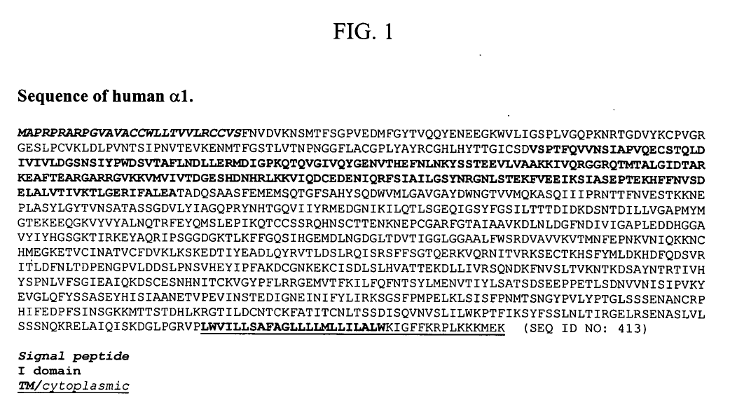

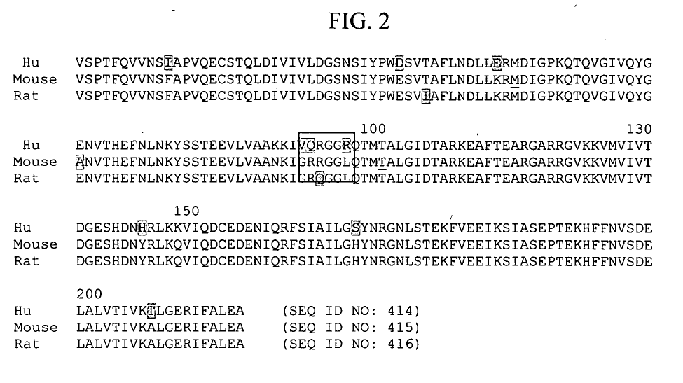

In some aspects, the present invention relates to the generation and identification of isolated antibodies that bind to Very Late Antigen-1 (VLA-1)and preferably the alpha subunit of VLA-1 (the amino acid sequence of the alpha 1 subunit (“axl” or “CD49a”)). The sequence of an alpha 1 subunit of VLA-1 is shown in FIG. 1 and an alignment between the I domains of different alpha 1 subunits of VLA-1 from various organisms is shown in FIG. 2. The antibodies to these alpha 1 subunits are fully human, monoclonal antibodies that bind to VLA-1 with high affinity, high potency, or both high affinity and high potency. Accordingly, some embodiments of the present invention provide isolated antibodies, or fragments of those antibodies, that bind to VLA-1.

In some aspects, the antibodies are specific for human VLA-1, for example, the antibodies will not bind to VLA-2 and/or mouse VLA-1.

In some aspects, the antibodies to VLA-1 are particularly potent antibodies (e.g., antibodies with a very low IC₅₀) or particularly high affinity antibodies (e.g., antibodies with a very low K_{D}).

In some aspects, the antibodies to VLA-1 bind to particular epitopes on VLA-1, which can include amino acid positions E66, K142, S162, and some combination thereof.

In some aspects, the antibodies described herein are capable of preventing the adhesion of VLA-1 to collagen. VLA-1 and VLA-2, collagen/laminin receptors on T cells, are not expressed on naive T cells, but are upregulated on activated T cells and remain high on a subset of memory cells long after the stimulus has presumably disappeared (Riikonen, et al., Biochem Biophys Res Commun 209:205-212 (1995)). One mechanism of attenuating an undesired T cell-dependent inflammatory or autoimmune response is to inhibit their retention in inflamed tissue by blocking their ability to interact with the surrounding interstitial matrix. Thus, in some embodiments the antibodies that are able to prevent adhesion of VLA-1 to collagen are used to block the VLA-1-matrix interactions as an approach to treating human immune disorders.

VLA-1 related (or mediated) disorder can include inflammatory disorders, such as immune-mediated inflammatory disorders (IMIDs), which are inflammatory conditions caused and sustained by an antigen-specific, pathological immune response. Among these disorders are various types of arthritis, such as rheumatoid arthritis, as well as allergic diseases, such as asthma, hay fever, and urticaria; different types of connective tissue disorders; inflammatory bowel disease (e.g., Crohn's disease and ulcerative colitis); insulin-dependent diabetes; uveitis; retinitis; graft rejection; and graft-versus host-disease. VLA-1 related disorders can also include tissue inflammation in infectious, ischemic, hemorrhagic, and traumatic conditions, e.g., fasciitis, stroke, infarction of the myocardium and other organs (e.g., lung and intestine), ARDS; hepatitis, (e.g., infectious and non-infectious, acute and chronic); acute and chronic pancreatitis; reperfusion injuries; radiation injuries; vascular restenosis of different types (e.g., coronary restenosis). Also included are osteoarthritis, osteoporosis, atherosclerosis, organ fibrosis, and neurodegenerative disorders such as Alzheimer's disease and multiple sclerosis. VLA-1 related disorders can include cancer, blood malignancies, e.g., leukemias and multiple myelomas; the development of a number of solid tumors, tumor growth, and metastatic spreading, or some combination thereof. In some embodiments, the VLA-1 related or mediated disorders is selected from the group of skin related conditions, allergic rhinitis, respiratory distress syndrome, bronchitis, tendinitis, bursitis, fever, migraine headaches, gastrointestinal conditions, periarteritis nodosa, thyroiditis, aplastic anemia, Hodgkin's Disease, rheumatic fever, autoimmune diseases, sarcoidosis, nephrotic syndrome, renal failure, Behcet's Syndrome, polymyositis, gingivitis, hypersensitivity, conjunctivitis, swelling occurring after injury, myocardial ischemia, endotoxin shock syndrome, renal scarring, and eosinophilic inflammation, or some combination thereof.

In association with such treatment, articles of manufacture comprising the antibodies described herein are also provided. Additionally, an assay kit comprising these antibodies is provided to screen for diseases or disorders associated with VLA-1 activity.

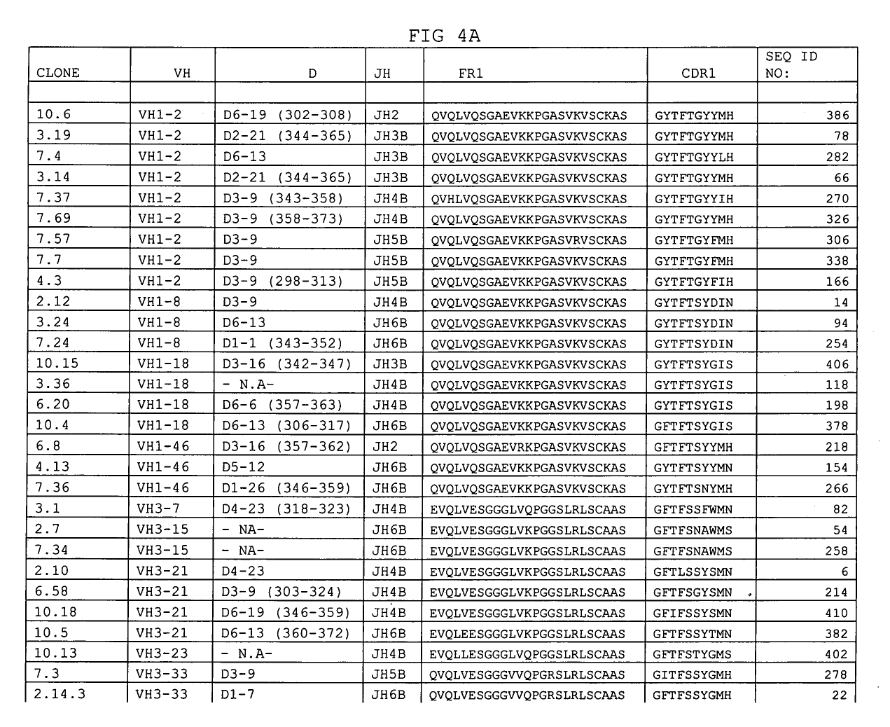

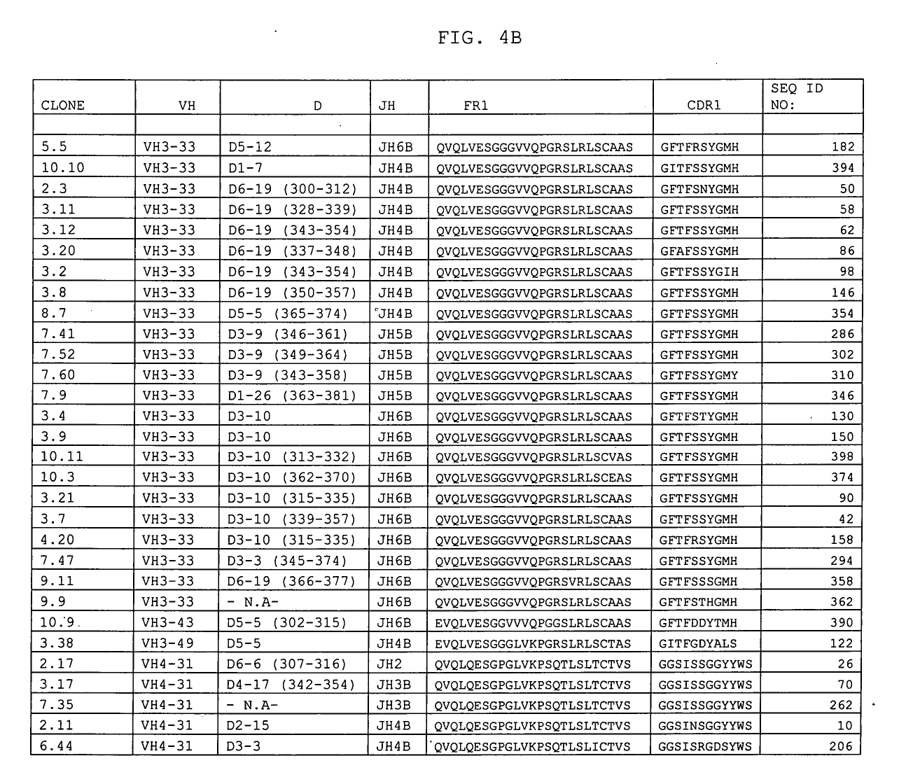

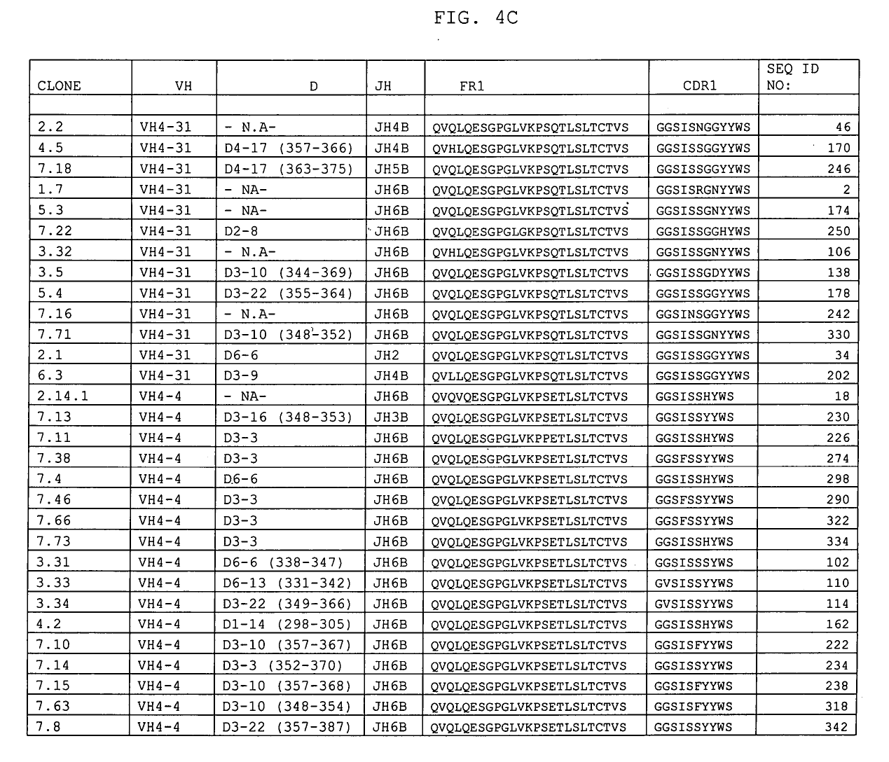

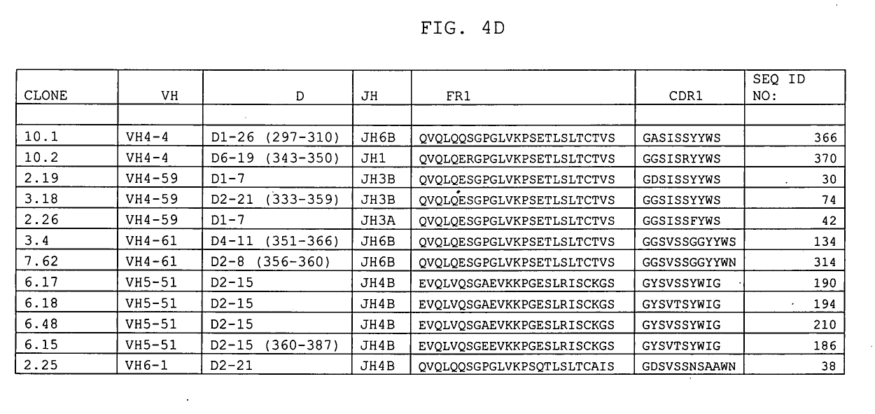

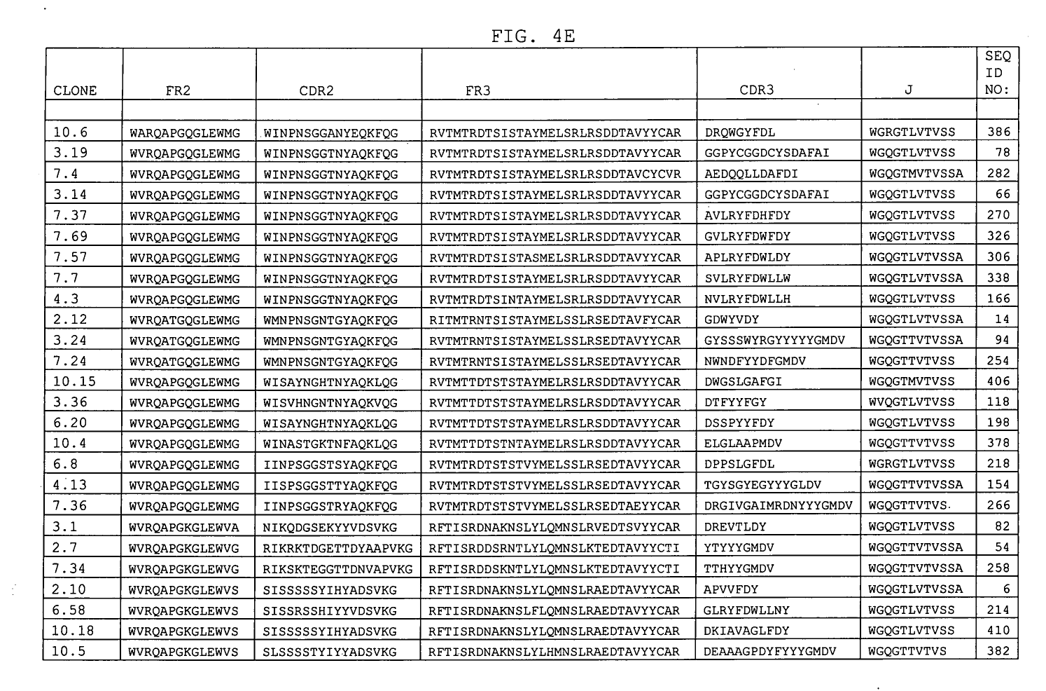

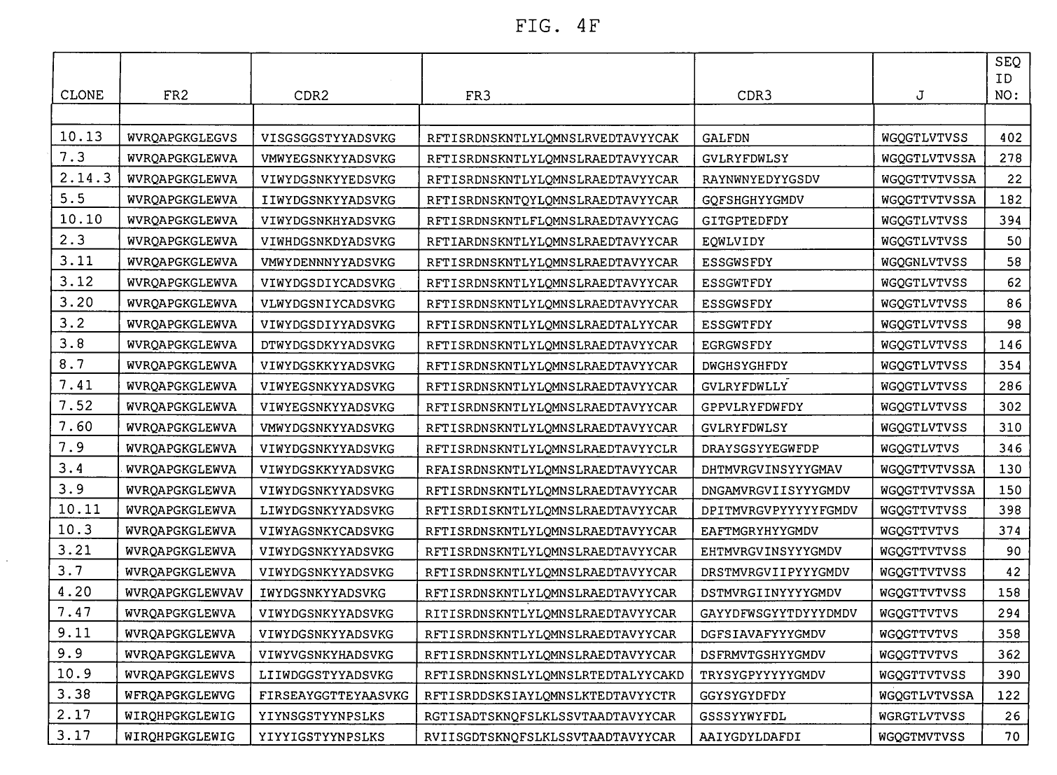

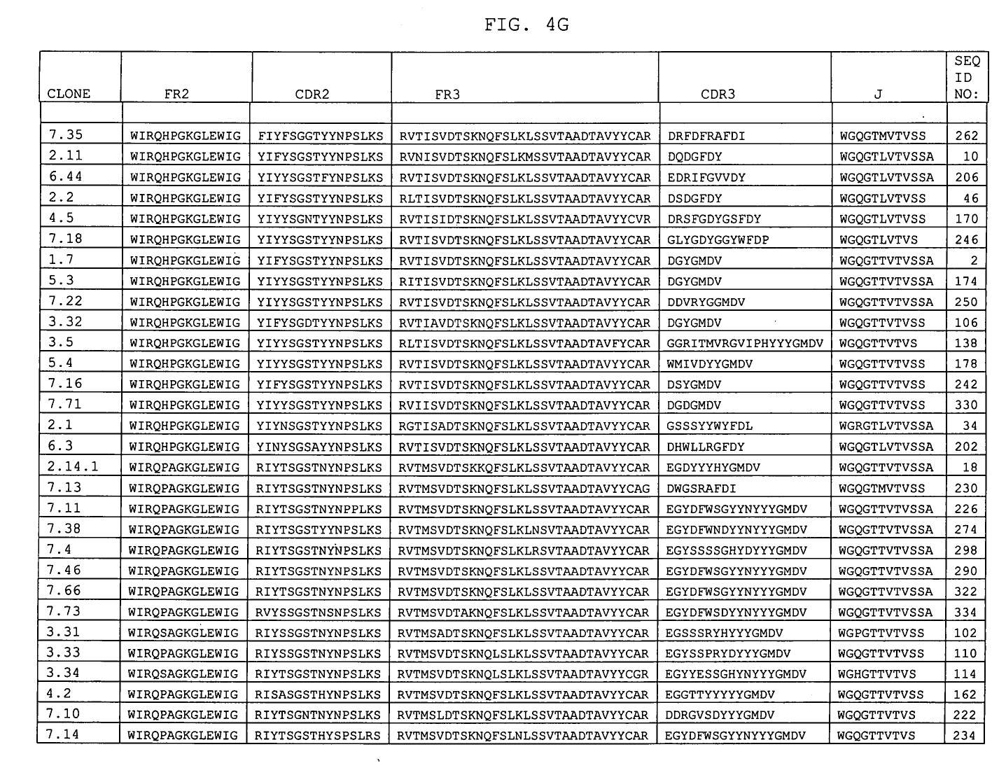

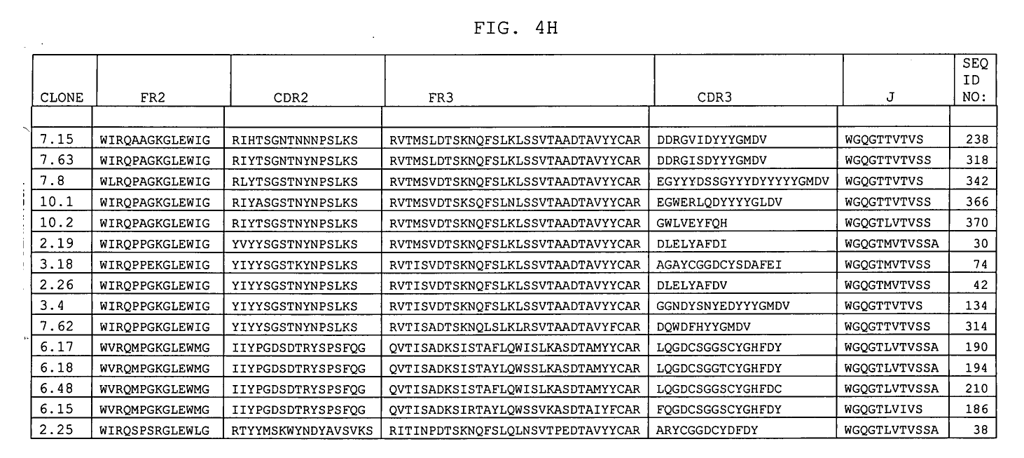

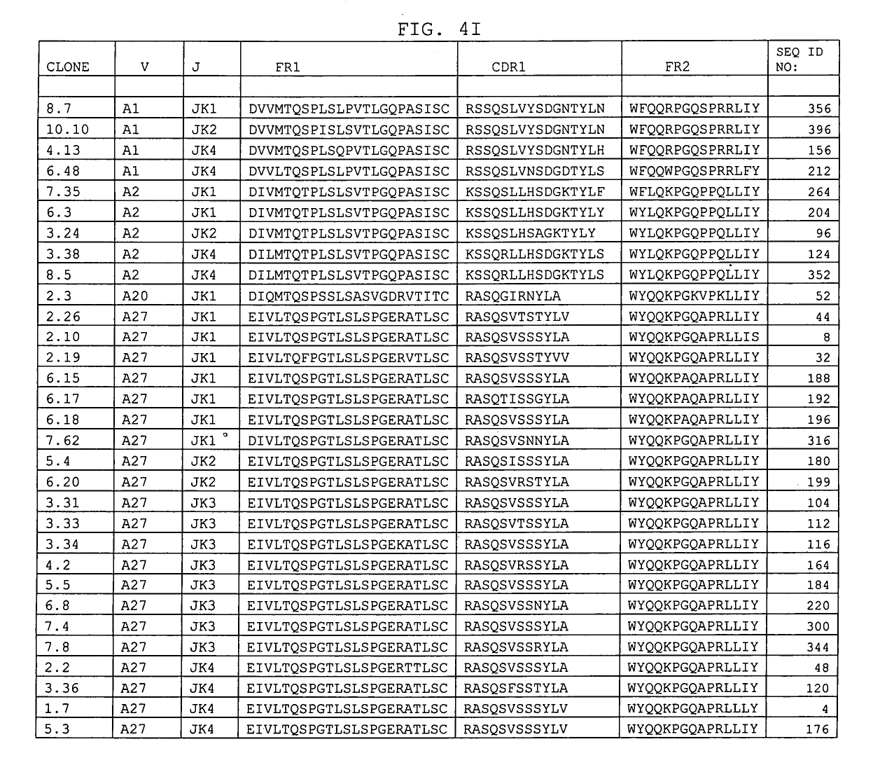

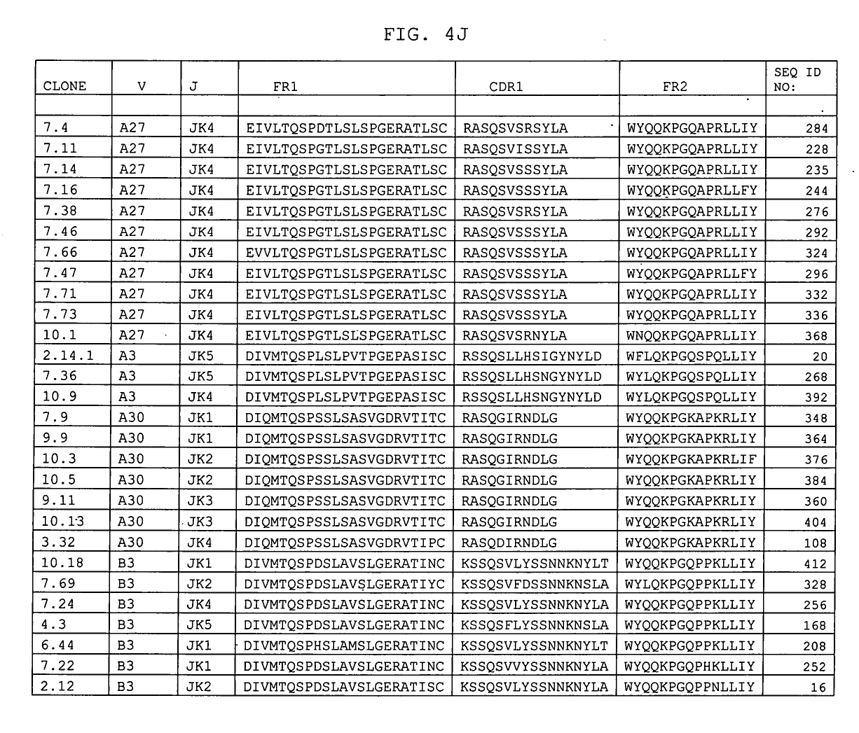

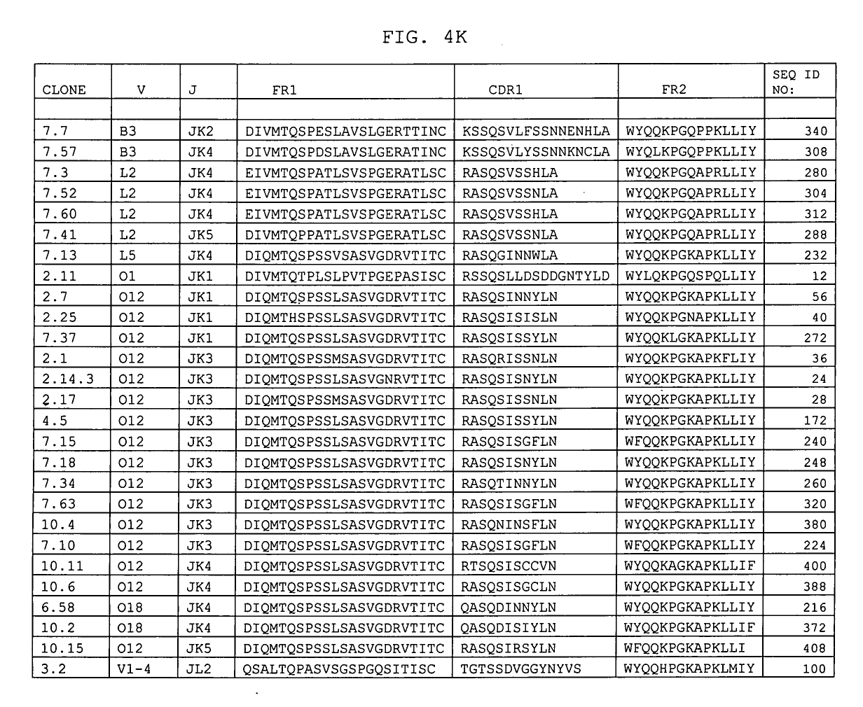

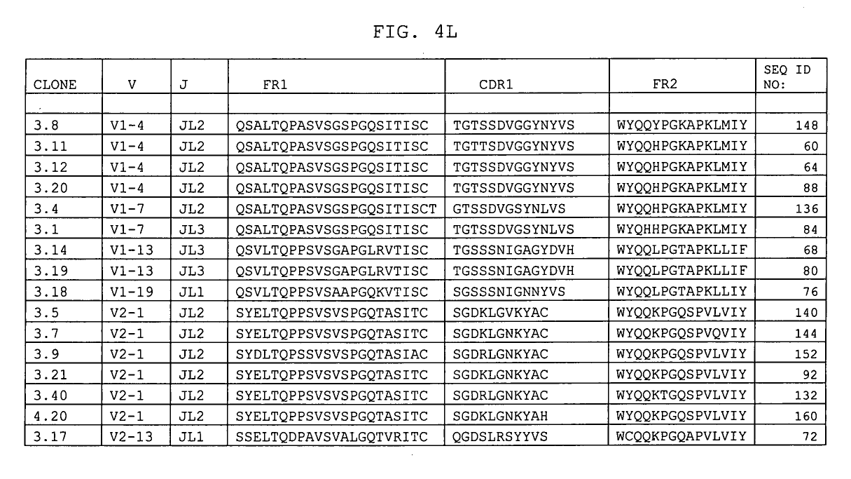

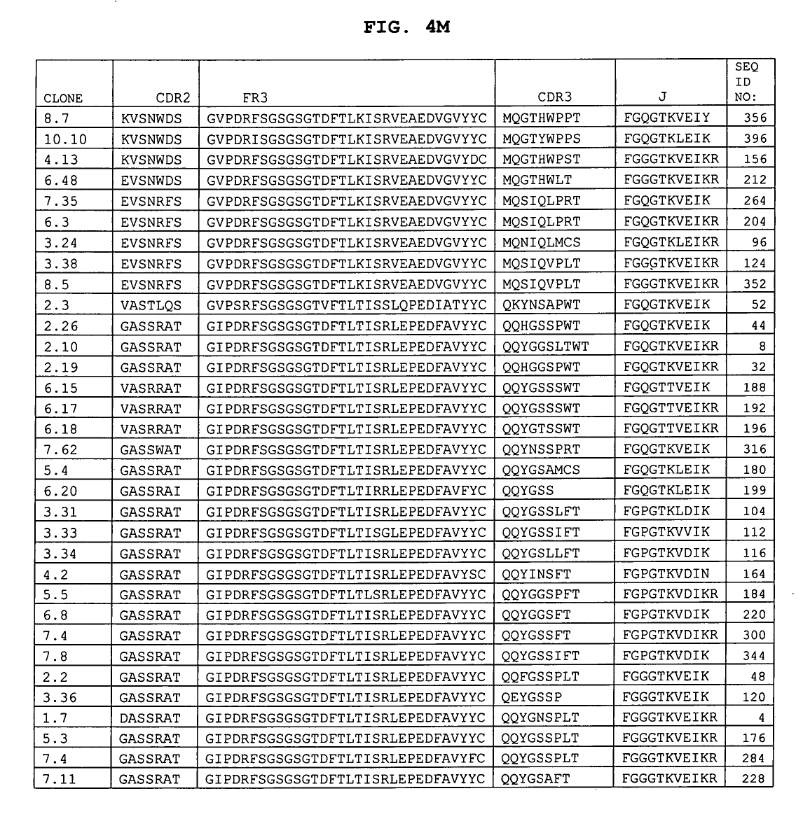

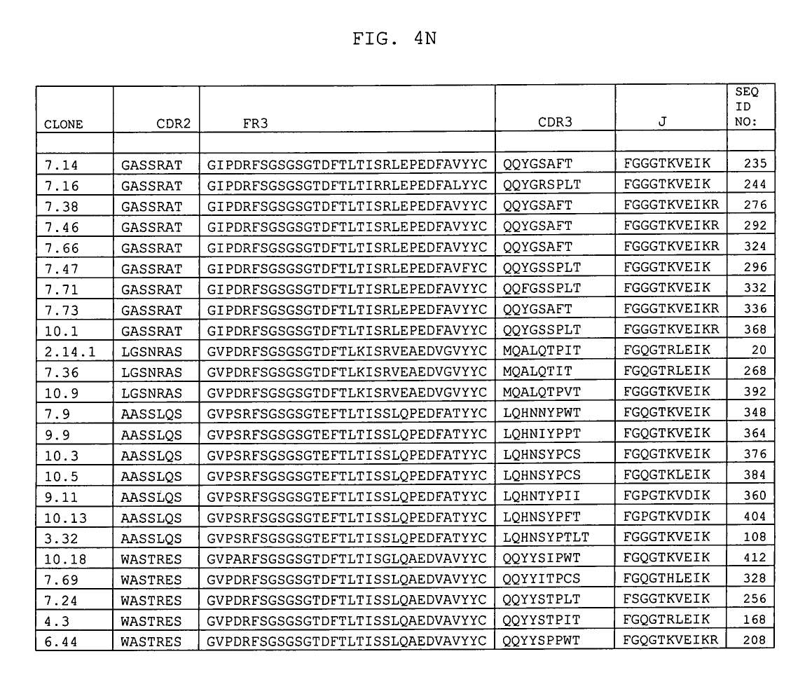

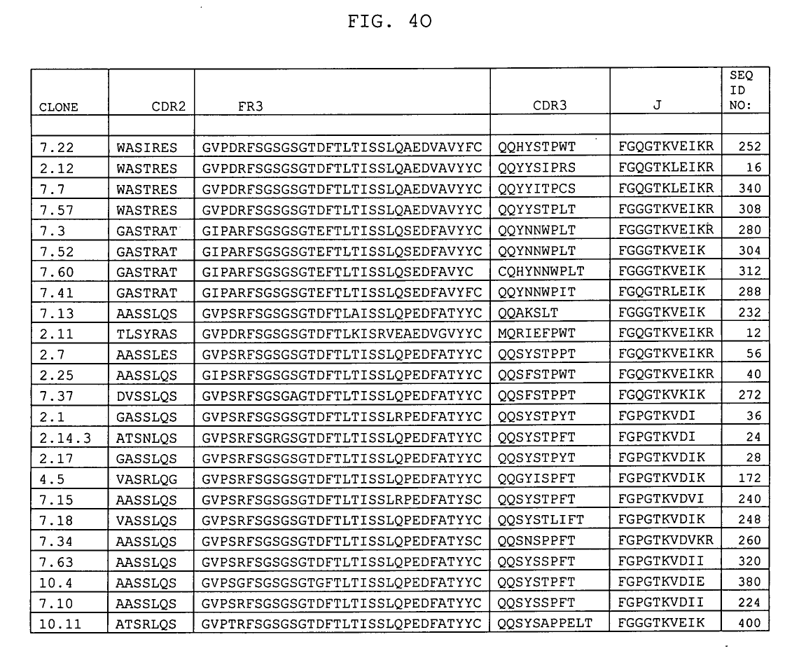

In some aspects, the antibody that binds to VLA-1 comprises a human heavy chain immunoglobulin molecule having an amino acid sequence shown in SEQ ID NOs: 2, 6, 10, 14, 18, 22, 26, 30, 34, 38, 42, 46, 50, 54, 58, 62, 66, 70, 74, 78, 82, 86, 90, 94, 98, 102, 106, 110, 114, 118, 122, 126, 130, 134, 138, 142, 146, 150, 154, 158, 162, 166, 170, 174, 178, 182, 186, 190, 194, 198, 202, 206, 210, 214, 218, 222, 226, 230, 234, 238, 242, 246, 250, 254, 258, 262, 266, 270, 274, 278, 282, 286, 290, 294, 298, 302, 306, 310, 314, 318, 322, 326, 330, 334, 338, 342, 346, 350, 354, 358, 362, 366, 370, 374, 378, 382, 386, 390, 394, 398, 402, 406, and 410. In another aspect, the antibody that binds to VLA-1 comprises a human kappa light chain immunoglobulin molecule having an amino acid sequence shown in SEQ ID NOs: 4, 8, 12, 16, 20, 24, 28, 32, 36, 40, 44, 48, 52, 56, 60, 64, 68, 72, 76, 80, 84, 88, 92, 96, 100, 104, 108, 112, 116, 120, 124, 128, 132, 136, 140, 144, 148, 152, 156, 160, 164, 168, 172, 176, 180, 184, 188, 192, 196, 200, 204, 208, 212, 216, 220, 224, 228, 232, 236, 240, 244, 248, 252, 256, 260, 264, 268, 272, 276, 280, 284, 288, 292, 296, 300, 304, 308, 312, 316, 320, 324, 328, 332, 336, 340, 344, 348, 352, 356, 360, 364, 368, 372, 376, 380, 384, 388, 392, 396, 400, 404, 408, and 412. In some embodiments, the antibody molecules are formed by combinations comprising the above recited heavy chain immunoglobulin molecules with the above recited light chain immunoglobulin molecules, such as the kappa light chain immunoglobulin molecules, and vice versa, as well as fragments and analogs thereof. In some embodiments, the antibody has a sequence from the heavy chain CDR1, CDR2, CDR3, FR1, FR2, FR3, and/or FR4 or any of the sequences listed in FIGS. 4A-4H and/or FIGS. 41-4P. In some embodiments, the antibody has a sequence from the light chain CDR1, CDR2, CDR3, FR1, FR2, and/or FR3 or any of the sequences listed FIGS. 4A-4H and/or FIGS. 4I-4P.

The nucleic acids described herein, and fragments and variants thereof, may be used, by way of nonlimiting example, (a) to direct the biosynthesis of the corresponding encoded proteins, polypeptides, fragments and variants as recombinant or heterologous gene products, (b) as probes for detection and quantification of the nucleic acids disclosed herein, (c) as sequence templates for preparing antisense molecules, and the like. Such uses are described more fully below.

### DEFINITIONS

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular.

Generally, nomenclatures utilized in connection with, and techniques of, cell and tissue culture, molecular biology, and protein and oligo- or polynucleotide chemistry and hybridization described herein are those well known and commonly used in the art, as described in various general and more specific references such as those that are cited and discussed throughout the present specification. See e.g. Singleton et al., Dictionary of Microbiology and Molecular Biology 2^{nd} ed., J. Wiley & Sons (New York, NY 1994); Sambrook et al. Molecular Cloning: A Laboratory Manual (2d ed., Cold Spring Harbor Laboratory Press, Cold Spring Harbor, N.Y. (1989)), which is incorporated herein by reference. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. Standard techniques are also used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

“Polymerase chain reaction” or “PCR” refers to a procedure or technique in which minute amounts of a specific piece of nucleic acid, RNA and/or DNA, are amplified as described in U.S. Pat. No. 4,683,195 issued Jul. 28, 1987. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers can coincide with the ends of the amplified material. PCR can be used to amplify specific RNA sequences, specific DNA sequences from total genomic DNA, and cDNA transcribed from total cellular RNA, bacteriophage or plasmid sequences, etc. See generally Mullis et al., Cold Spring Harbor Symp. Quant. Biol. 51:263 (1987); Erlich, ed., PCR Technology (Stockton Pres, NY, 1989). A used herein, PCR is considered to be one, but not the only, example of a nucleic acid polymerase reaction method for amplifying a nucleic acid test sample comprising the use of a known nucleic acid as a primer and a nucleic acid polymerase to amplify or generate a specific piece of nucleic acid.

“Antibodies” (Abs) and “immunoglobulins” (Igs) are glycoproteins having the same structural characteristics. While antibodies exhibit binding specificity to a specific antigen, immunoglobulins include both antibodies and other antibody-like molecules which lack antigen specificity. Polypeptides of the latter kind are, for example, produced at low levels by the lymph system and at increased levels by myelomas.

“Native antibodies and immunoglobulins” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light- and heavy-chain variable domains (Chothia et al. J. Mol. Biol. 186:651 (1985; Novotny and Haber, Proc. Natl. Acad. Sci. U.S.A. 82:4592 (1985); Chothia et al., Nature 342:877-883 (1989)).

The term “antibody” herein is used in the broadest sense and specifically covers intact monoclonal antibodies, polyclonal antibodies, multispecific antibodies (e.g. bispecific antibodies) formed from at least two intact antibodies, and antibody fragments including Fab and F(ab)′2, so long as they exhibit the desired biological activity. The “light chains” of antibodies (immunoglobulins) from any vertebrate species can be assigned to one of two clearly distinct types, called κ and λ, based on the amino acid sequences of their constant domains. Binding fragments are produced by recombinant DNA techniques, or by enzymatic or chemical cleavage of intact antibodies. Binding fragments include Fab, Fab′, F(ab′)₂, Fv, and single-chain antibodies, as described in more detail below. An antibody other than a “bispecific” or “bifunctional” antibody is understood to have each of its binding sites identical.

Depending on the amino acid sequence of the constant domain of their heavy chains, intact antibodies can be assigned to different “classes.” There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al, Nature, 352:624-628 (1991) and Marks et al., J. Mol. Biol., 222:581-597 (1991), for example.

An “isolated” antibody is one that has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

A “neutralizing antibody” is an antibody molecule which is able to eliminate or significantly reduce an effector function of a target antigen to which it binds. Accordingly, a “neutralizing” VLA-1 antibody is capable of eliminating or significantly reducing an effector function, such as VLA-1 activity (e.g., binding to collagen). In one embodiment, a neutralizing antibody will reduce an effector function by 1-10, 10-20, 20-30, 30-50, 50-70, 70-80, 80-90, 90-95, 95-99, or 99-100%. In another embodiment, this is measured in terms of potency or IC₅₀, discussed in more detail below.

“Antibody-dependent cell-mediated cytotoxicity” and “ADCC” refer to a cell-mediated reaction in which non-specific cytotoxic cells that express Ig Fc receptors (FcRs) (e.g. Natural Killer (NK) cells, neutrophils, and macrophages) recognize bound antibody on a target cell and subsequently cause lysis of the target cell. The primary cells for mediating ADCC, NK cells, express FcyRI only, whereas monocytes express FcγRI, FcγRII and FcγRIII. FcRs expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362, or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1988).

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the Ig light-chain and heavy-chain variable domains. The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies (see Kabat et al. (1991). The constant domains are not involved directly in binding an antibody to an antigen, but exhibit various effector functions, such as participation of the antibody in antibody-dependent cellular toxicity.

Digestion of antibodies with the enzyme, papain, results in two identical antigen-binding fragments, known also as “Fab” fragments, and a “Fc” fragment, having no antigen-binding activity but having the ability to crystallize. Digestion of antibodies with the enzyme, pepsin, results in the a F(ab′)₂ fragment in which the two arms of the antibody molecule remain linked and comprise two-antigen binding sites. The F(ab′)₂ fragment has the ability to crosslink antigen.

“Fv” when used herein refers to the minimum fragment of an antibody that retains both antigen-recognition and antigen-binding sites.

“Fab” when used herein refers to a fragment of an antibody which comprises the constant domain of the light chain and the CHI domain of the heavy chain.

“Fv” is the minimum antibody fragment which contains a complete antigen-recognition and binding site. In a two-chain Fv species, this region consists of a dimer of one heavy- and one light-chain variable domain in tight, non-covalent association. In a single-chain Fv species, one heavy- and one light-chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind, antigen, although at a lower affinity than the entire binding site.

The term “hypervariable region” when used herein refers to the amino acid residues of an antibody which are responsible for antigen-binding. The hypervariable region generally comprises amino acid residues from a “complementarity determining region” or “CDR” (e.g. residues 24-34 (Li), 50-62 (L2), and 89-97 (L3) in the light chain variable domain and 31-55 (H1), 50-65 (H2) and 95-102 (H3) in the heavy chain variable domain; Kabat et al., Sequences of Proteins of Immunological Interest, 5^{th} Ed. Public Health Service, National Institutes of Health, Bethesda, Md. (1991)) and/or those residues from a “hypervariable loop”(e.g. residues 26-32 (L1), 50-52 (L2) and 91-96 (L3) in the light chain =variable domain and 26-32 ((H1), 53-55 (H2) and 96-101 (H3) in the heavy chain variable domain; Chothia and Lesk J. Mol. Biol. 196:901-917 (1987)). “Framework Region” or “FR” residues are those variable domain residues other than the hypervariable region residues as herein defined.

The term “complementarity determining regions” or “CDRs” when used herein refers to parts of immunological receptors that make contact with a specific ligand and determine its specificity. The CDRs of immunological receptors are the most variable part of the receptor protein, giving receptors their diversity, and are carried on six loops at the distal end of the receptor's variable domains, three loops coming from each of the two variable domains of the receptor.

The term “epitope” is used to refer to binding sites for antibodies on protein antigens. Epitopic determinants usually consist of chemically active surface groupings of molecules such as amino acids or sugar side chains and usually have specific three dimensional structural characteristics, as well as specific charge characteristics. An antibody is said to bind an antigen when the dissociation constant is ≦1 μM, preferably ≦100 nM and most preferably ≦10 nM. An increased or greater dissociation constant (“K_{d}” or “KD” or “K_{D}”) means that there is less affinity between the epitope and the antibody. In, other words, that the antibody and the epitope are less favorable to bind or stay bound together. A decrease of lower dissociation constant means that there is a higher affinity between the epitope and the antibody. In other words, it is more likely that the antibody and the epitope will bind or stay bound together. An antibody with a K_{D} of “no more than” a certain amount means that the antibody will bind to the epitope at least that well or more strongly (or tightly).

While K_{D} describes the binding characteristics of an epitope and an antibody, “potency” describes the effectiveness of the antibody itself for a function of the antibody. A relatively low K_{D} does not automatically mean a high potency. Thus, antibodies can have a relatively low K_{D} and a high potency (e.g., they bind well and alter the function strongly), a relatively high K_{D} and a high potency (e.g., they do not bind well but have a strong impact on function), a relatively low K_{D} and a low potency (e.g., they bind well, but not in a manner effective to alter a particular function) or a relatively high K_{D} and a low potency (e.g., they do not bind to the target well, or do not significantly impact function). In one embodiment, high potency means that there is a high level of inhibition with a low concentration of antibody. In one embodiment, an antibody is potent or has a high potency when its IC₅₀ is a small value, for example, 100-70 nM, 70-30 nM, 30-10 nM, 10-1 nM, 1000-500 pM, 500-100 pM, 100-90 pM, 90-60 pM, 60-30, 30-20, 20-10 pM or less. As will be appreciated by one of skill in the art, the IC₅₀s or K_{D}s can be determined under various conditions, and similar conditions should be used for comparison between antibodies. For example, any of the present antibodies can have their K_{D} S and IC₅₀ measured with or without Mg²⁺, or with a particular amount of Mg²⁺. In other embodiments, EC₅₀s can be used.

“Substantially,” unless otherwise specified in conjunction with another term, means that the value can vary within any amount that is contributable to errors in measurement that may occur during the creation or practice of the embodiments. “Significant” means that the value can vary as long as it is sufficient to allow the invention to function for its intended use.

The term “selectively bind” in reference to an antibody does not mean that the antibody only binds to a single substance. Rather, it denotes that the K_{D} of the antibody to a first substance is less than the K_{D} of the antibody to a second substance. Antibodies which exclusively bind to an epitope only bind to that single epitope.

The term “amino acid” or “amino acid residue,” as used herein, refers to naturally occurring L amino acids or to D amino acids as described further below with respect to variants. The commonly used one and three-letter abbreviations for amino acids are used herein (Bruce Alberts et al., Molecular Biology of the Cell, Garland Publishing, Inc., New York (3d ed. 1994)).

The term “mAb” refers to monoclonal antibody.

The term “XENOMOUSE®” refers to strains of mice which have been engineered to contain 245 kb and 190 kb-sized germline configuration fragments of the human heavy chain locus and kappa light chain locus, as described in Green et al. Nature Genetics 7:13-21 (1994), incorporated herein by reference. Other XenoMouse strains of mice contain 980 kb and 800 kb-sized germline configuration fragments of the human heavy chain locus and kappa light chain locus. Still other XenoMouse strains of mice contain 980 kb and 800 kb-sized germline configuration fragments of the human heavy chain locus and kappa light chain locus plus a 740 kb-sized germline configured complete human lambda light chain locus. See Mendez et al. Nature Genetics 15:146-156 (1997) and Green and Jakobovits J. Exp. Med. 188:483-495 (1998). The XENOMOUSE® strains are available from Abgenix, Inc. (Fremont, Calif.).

The term “XENOMAX™” refers use of to the use of the “Selected Lymphocyte Antibody Method” (Babcook et al., Proc. Natl. Acad. Sci. USA, i93:7843-7848 (1996)), when used with XENOMOUSE® animals.

The term “SLAM®” refers to the “Selected Lymphocyte Antibody Method” (Babcook et al., Proc. Natl. Acad. Sci. USA, i93:7843-7848 (1996), and Schrader, U.S. Pat. No. 5,627,052), both of which are incorporated by reference in their entireties.

The terms “disease,” “disease state” and “disorder” refer to a physiological state of a cell or of a whole mammal in which an interruption, cessation, or disorder of cellular or body functions, systems, or organs has occurred.

The term “symptom” means any physical or observable manifestation of a disorder, whether it is generally characteristic of that disorder or not. The term “symptoms” can mean all such manifestations or any subset thereof.

The term “treat” or “treatment” refer to both therapeutic treatment and prophylactic or preventative measures, wherein the object is to prevent or slow down (lessen) an undesired physiological change or disorder, such as the development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented. The term “inhibit,” when used in conjunction with a disease or symptom can mean that the antibody can reduce or eliminate the disease or symptom.

The term “patient” includes human and veterinary subjects.

“Administer,” for purposes of treatment, means to deliver to a patient. For example and without limitation, such delivery can be intravenous, intraperitoneal, by inhalation, intramuscular, subcutaneous, oral, topical, transdermal, or surgical.

“Therapeutically effective amount,” or dose, for purposes of treatment, means an amount such that an observable change in the patient's condition and/or symptoms could result from its administration, either alone or in combination with other treatment. A unit dose is an amount to be given in a single administration to provide treatment for a desired amount of time or under a particular set of conditions. The unit dose can be in a form that allows ready administration of the antibody.

A “pharmaceutically acceptable vehicle,” for the purposes of treatment, is a physical embodiment that can be administered to a patient. Pharmaceutically acceptable vehicles can be, but are not limited to, pills, capsules, caplets, tablets, orally administered fluids, injectable fluids, sprays, aerosols, lozenges, neutraceuticals, creams, lotions, oils, solutions, pastes, powders, vapors, or liquids. One example of a pharmaceutically acceptable vehicle is a buffered isotonic solution, such as phosphate buffered saline (PBS).

“Neutralize,” for purposes of treatment, means to partially or completely suppress chemical and/or biological activity.

“Down-regulate,” for purposes of treatment, means to lower the level of a particular target composition.

“Mammal” for purposes of treatment refers to any animal classified as a mammal, including humans, domestic and farm animals, and zoo, sports, or pet animals, such as monkeys, dogs, horses, cats, cows, etc.

The term “polynucleotide” as referred to herein means a polymeric form of nucleotides of at least 10 bases in length, either ribonucleotides or deoxynucleotides or a modified form of either type of nucleotide. The term includes single and double stranded forms of DNA.

The term “isolated polynucleotide” as used herein shall mean a polynucleotide of genomic, cDNA, or synthetic origin or some combination thereof, which by virtue of its origin the “isolated polynucleotide” (1) is not associated with all or a portion of a polynucleotide in which the “isolated polynucleotide” is found in nature, (2) is operably linked to a polynucleotide which it is not linked to in nature, or (3) does not occur in nature as part of a larger sequence.

The term “oligonucleotide” referred to herein includes naturally occurring, and modified nucleotides linked together by naturally occurring, and non-naturally occurring oligonucleotide linkages. Oligonucleotides are a polynucleotide subset generally comprising a length of 200 bases or fewer. Preferably, oligonucleotides are 10 to 60 bases in length and most preferably 12, 13, 14, 15, 16, 17, 18, 19, or 20 to 40 bases in length. Oligonucleotides are usually single stranded, e.g., for probes; although oligonucleotides may be double stranded, e.g., for use in the construction of a gene mutant. Oligonucleotides can be either sense or antisense oligonucleotides.

The term “naturally occurring nucleotide” as used herein includes deoxyribonucleotides and ribonucleotides. The term “modified nucleotides” referred to herein includes nucleotides with modified or substituted sugar groups and the like. The term oligonucleotide linkages” referred to herein includes oligonucleotides linkages such as phosphorothioate, phosphorodithioate, phosphoroselenoate, phosphorodiselenoate, phosphoroanilothioate, phosphoraniladate, phosphoroamidate, and the like. See e.g., LaPlanche et al. Nucl. Acids Res. 14:9081 (1986); Stec et al. J. Am. Chem. Soc. 106:6077 (1984); Stein et al. Nucl. Acids Res. 16:3209 (1988); Zon et al. Anti-Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990), the disclosures of which are hereby incorporated by reference. An oligonucleotide can include a label for detection, if desired.

The term “selectively hybridize” referred to herein means to detectably and specifically bind. Polynucleotides, oligonucleotides and fragments thereof selectively hybridize to nucleic acid strands under hybridization and wash conditions that minimize appreciable amounts of detectable binding to nonspecific nucleic acids. High stringency conditions can be used to achieve selective hybridization conditions as known in the art and discussed herein. Generally, the nucleic acid sequence homology between the polynucleotides, oligonucleotides, or antibody fragments and a nucleic acid sequence of interest will be at least 80%, and more typically with preferably increasing homologies of at least 85%, 90%, 95%, 99%, and 100%.

The term “control sequence” as used herein refers to polynucleotide sequences which are necessary to effect the expression and processing of coding sequences to which they are connected. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences.

The term “operably linked” as used herein refers to positions of components so described that are in a relationship permitting them to function in their intended manner. For example, a control sequence “operably linked” to a coding sequence is connected in such a way that expression of the coding sequence is achieved under conditions compatible with the control sequences.

The term “isolated protein” referred to herein means a protein of cDNA, recombinant RNA, or synthetic origin or some combination thereof, which by virtue of its origin, or source of derivation, the “isolated protein” (1) is not associated with proteins found in nature, (2) is free of other proteins from the same source, e.g., free of murine proteins, (3) is expressed by a cell from a different species, or (4) does not occur in nature.

The term “polypeptide” is used herein as a generic term to refer to native protein, fragments, or analogs of a polypeptide sequence. Hence, native protein, fragments, and analogs are species of the polypeptide genus. Preferred polypeptides in accordance with the invention comprise the human heavy chain immunoglobulin molecules represented by SEQ ID NOs: 2, 6, 10, 14, 18, 22, 26, 30, 34, 38, 42, 46, 50, 54, 58, 62, 66, 70, 74, 78, 82, 86, 90, 94, 98, 102, 106, 110, 114, 118, 122, 126, 130, 134, 138, 142, 146, 150, 154, 158, 162, 166, 170, 174, 178, 182, 186, 190, 194, 198, 202, 206, 210, 214, 218, 222, 226, 230, 234, 238, 242, 246, 250, 254, 258, 262, 266, 270, 274, 278, 282, 286, 290, 294, 298, 302, 306, 310, 314, 318, 322, 326, 330, 334, 338, 342, 346, 350, 354, 358, 362, 366, 370, 374, 378, 382, 386, 390, 394, 398, 402, 406, and 410, for example, and the human kappa light chain immunoglobulin molecules represented by SEQ ID NOs: 4, 8, 12, 16, 20, 24, 28, 32, 36, 40, 44, 48, 52, 56, 60, 64, 68, 72, 76, 80, 84, 88, 92, 96, 100, 104, 108, 112, 116, 120, 124, 128, 132, 136, 140, 144, 148, 152, 156, 160, 164, 168, 172, 176, 180, 184, 188, 192, 196, 200, 204, 208, 212, 216, 220, 224, 228, 232, 236, 240, 244, 248, 252, 256, 260, 264, 268, 272, 276, 280, 284, 288, 292, 296, 300, 304, 308, 312, 316, 320, 324, 328, 332, 336, 340,344, 348, 352, 356, 360, 364, 368, 372, 376, 380, 384, 388, 392, 396, 400, 404, 408, and 412, for example, as well as antibody molecules formed by combinations comprising the heavy chain immunoglobulin molecules with light chain immunoglobulin molecules, such as the kappa light chain immunoglobulin molecules, and vice versa, as well as fragments and analogs thereof.

Unless specified otherwise, the left-hand end of single-stranded polynucleotide sequences is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA and which are 5′ to the 5′ end of the RNA transcript are referred to as “upstream sequences”; sequence regions on the DNA strand having the same sequence as the RNA and which are 3′ to the 3′ end of the RNA transcript are referred to as “downstream sequences”.

As used herein, the twenty conventional amino acids and their abbreviations follow conventional usage. See Immunology—A Synthesis (2nd Edition, E. S. Golub and D. R. Gren, Eds., Sinauer Associates, Sunderland, Mass. (1991)), which is incorporated herein by reference. Stereoisomers (e.g., D-amino acids) of the twenty conventional amino acids, unnatural amino acids such as alpha-, alpha-disubstituted amino acids, N-alkyl amino acids, lactic acid, and other unconventional amino acids may also be suitable components for polypeptides of the present invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the lefthand direction is the amino terminal direction and the righthand direction is the carboxy-terminal direction, in accordance with standard usage and convention.

The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence.

In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The following terms are among those used to describe the sequence relationships between two or more polynucleotide or amino acid sequences: “reference sequence”, “comparison window”, “sequence identity”, “percentage of sequence identity”, “substantial identity”, and “homology.” A “reference sequence” is a defined sequence used as a basis for a sequence comparison. A reference sequence may be a subset of a larger sequence, for example, as a segment of a full-length cDNA or gene sequence given in a sequence listing or may comprise a complete cDNA or gene sequence. Generally, a reference sequence is at least 18 nucleotides or 6 amino acids in length, frequently at least 24 nucleotides or 8 amino acids in length, and often at least 48 nucleotides or 16 amino acids in length. Since two polynucleotides or amino acid sequences may each (1) comprise a sequence (i.e., a portion of the complete polynucleotide or amino acid sequence) that is similar between the two molecules, and (2) may further comprise a sequence that is divergent between the two polynucleotides or amino acid sequences, sequence comparisons between two (or more) molecules are typically performed by comparing sequences of the two molecules over a “comparison window” to identify and compare local regions of sequence similarity.

A “comparison window”, as used herein, refers to a conceptual segment of at least about 18 contiguous nucleotide positions or about 6 amino acids wherein the polynucleotide sequence or amino acid sequence is compared to a reference sequence of at least 18 contiguous nucleotides or 6 amino acid sequences and wherein the portion of the polynucleotide sequence in the comparison window may include additions, deletions, substitutions, and the like (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman Adv. Appl. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85:2444 (1988), by computerized implementations of these algorithms (GAP, BESTFIT, FASTA, and TFASTA in the Wisconsin Genetics Software Package Release 7.0, (Genetics Computer Group, 575 Science Dr., Madison, Wis.), GENEWORKS™, or MACVECTOR® software packages), or by inspection, and the best alignment (i.e., resulting in the highest percentage of homology over the comparison window) generated by the various methods is selected.

The term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U, or I) or amino acid residue occurs in both sequences to yield the number of matched positions, dividing the number of matched positions by the total number of positions in the comparison window (i.e., the window size), and multiplying the result by 100 to yield the percentage of sequence identity. The terms “substantial identity” as used herein denotes a characteristic of a polynucleotide or amino acid sequence, wherein the polynucleotide or amino acid comprises a sequence that has at least 85 percent sequence identity, preferably at least 90 to 95 percent sequence identity, more preferably at least 99 percent sequence identity, as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which may include deletions or additions which total 20 percent or less of the reference sequence over the comparison window. The reference sequence may be a subset of a larger sequence.

Two amino acid sequences or polynucleotide sequences are “homologous” if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least about 30 amino acids in length) are homologous, as this term is used herein, if they have an alignment score of at more than 5 (in standard deviation units) using the program ALIGN with the mutation data matrix and a gap penalty of 6 or greater. See Dayhoff, M. O., in Atlas of Protein Sequence and Structure, pp. 101-110 (Volume 5, National Biomedical Research Foundation (1972)) and Supplement 2 to this volume, pp. 1-10. The two sequences or parts thereof are more preferably homologous if their amino acids are greater than or equal to 50% identical when optimally aligned using the ALIGN program.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity, and most preferably at least 99 percent sequence identity. Preferably, residue positions which are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, glutamic-aspartic, and asparagine-glutamine.

As discussed herein, minor variations in the amino acid sequences of antibodies or immunoglobulin molecules are contemplated as being encompassed by the present invention, providing that the variations in the amino acid sequence maintain at least 75%, more preferably at least 80%, 90%, 95%, and most preferably 99%. In particular, conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) non-polar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are described in detail herein. Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). The foregoing examples demonstrate that those of skill in the art can recognize sequence motifs and structural conformations that may be used to define structural and functional domains in accordance with the invention.

Preferred amino acid substitutions are those which: (1) reduce susceptibility to proteolysis, (2) reduce susceptibility to oxidation, (3) alter binding affinity for forming protein complexes, (4) alter binding affinities, and (4) confer or modify other physiocochemical or functional properties of such analogs. Analogs can include various muteins of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et at. Nature 354:105 (1991), which are each incorporated herein by reference.

The term “polypeptide fragment” as used herein refers to a polypeptide that has an amino-terminal and/or carboxy-terminal deletion, but where the remaining amino acid sequence is identical to the corresponding positions in the naturally-occurring sequence deduced, for example, from a full-length cDNA sequence. Fragments typically are at least 5, 6, 8 or 10 amino acids long, preferably at least 14 amino acids long, more preferably at least 20 amino acids long. In other embodiments polypeptide fragments are at least 25 amino acids long, more preferably at least 50 amino acids long, and even more preferably at least 70 amino acids long.

Peptide analogs are commonly used in the pharmaceutical industry as non-peptide drugs with properties analogous to those of the template peptide. These types of non-peptide compound are termed “peptide mimetics” or “peptidomimetics”. Fauchere, J. Adv. Drug Res. 15:29 (1986); Veber and Freidinger TINS p. 392 (1985); and Evans et al. J. Med. Chem. 30:1229 (1987), which are incorporated herein by reference. Such compounds are often developed with the aid of computerized molecular modeling. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a biochemical property or pharmacological activity), such as human antibody, but have one or more peptide linkages optionally replaced by a linkage selected from the group consisting of: —CH₂NH—, —CH₂S—, —CH₂—CH₂—, —CH═CH-(cis and trans), —COCH₂—, —CH(OH)CH₂—, and —CH₂SO—, by methods well known in the art. Systematic substitution of one or more amino acids of a consensus sequence with a D-amino acid of the same type (e.g., D-lysine in place of L-lysine) may be used to generate more stable peptides. In addition, constrained peptides comprising a consensus sequence or a substantially identical consensus sequence variation may be generated by methods known in the art (Rizo and Gierasch Ann. Rev. Biochem. 61:387 (1992), incorporated herein by reference); for example, by adding internal cysteine residues capable of forming intramolecular disulfide bridges which cyclize the peptide.

As used herein, the terms “label” or “labeled” refers to incorporation of a detectable marker, e.g., by incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). In certain situations, the label or marker can also be therapeutic. Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, P-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance.

The term “pharmaceutical agent or drug” as used herein refers to a chemical compound or composition capable of inducing a desired therapeutic effect when properly administered to a patient. Other chemistry terms herein are used according to conventional usage in the art, as exemplified by The McGraw-Hill Dictionary of Chemical Terms (Parker, S., Ed., McGraw-Hill, San Francisco (1985)), incorporated herein by reference).

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

VLA-1 proteins and nucleic acids encoding these proteins are known in the art. VLA-1 comprises an alpha subunit, alpha 1, and a beta subunit, beta 1. Sequences and alignments of the alpha 1 subunit are displayed in FIG. 1 and FIG. 2. Alternative names for VLA-1 include alpha 1/beta 1, α1/β1, and CD49a/CD29. Alternative names of the alpha 1 subunit include CD49a, α1, integrin α1, and Itga1.

The bulk of this specification directly addresses antibodies that bind to VLA-1; however, as will be appreciated by one of skill in the art, and as discussed in more detail below, the antibodies need not be raised against the alpha 1 subunit alone. For example, antibodies can be directed against the alpha 1/beta 1 interface, or to epitopes on the beta 1 subunit that are present only when the beta 1 subunit is bound to the alpha 1 subunit.

Antibody Structure

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site.

Thus, an intact antibody has two binding sites. Except in bifunctional or bispecific antibodies, the two binding sites are the same.

The chains all exhibit the same general structure of relatively conserved framework regions (FR) joined by three hyper variable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987); Chothia et al. Nature 342:878-883 (1989).

A bispecific or biftnctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann Clin. Exp. Immunol. 79: 315-321 (1990), Kostelny et al. J. Immunol. 148:1547-1553 (1992). Production of bispecific antibodies can be a relatively labor intensive process compared with production of conventional antibodies and yields and degree of purity are generally lower for bispecific antibodies. Bispecific antibodies do not exist in the form of fragments having a single binding site (e.g., Fab, Fab′, and Fv).

Human Antibodies and Humanization of Antibodies

Human antibodies avoid some of the problems associated with antibodies that possess murine or rat variable and/or constant regions. The presence of such murine or rat derived proteins can lead to the rapid clearance of the antibodies or can lead to the generation of an immune response against the antibody by a patient. In order to avoid the utilization of murine or rat derived antibodies, fully human antibodies can be generated through the introduction of human antibody function into a rodent so that the rodent produces fully human antibodies.

One method for generating fully human antibodies is through the use of XENOMOUSE® strains of mice which have been engineered to contain 245 kb and 190 kb-sized germline configuration fragments of the human heavy chain locus and kappa light chain locus. See Green et al. Nature Genetics 7:13-21 (1994). Other XenoMouse strains of mice contain 980 kb and 800 kb-sized germline configuration fragments off the human heavy chain locus and kappa light chain locus. Still other XenoMouse strains of mice contain 980 kb and 800 kb-sized germline configuration fragments of the human heavy chain locus and kappa light chain locus plus a 740 kb-sized germline configured complete human lambda light chain locus. See Mendez et al. Nature Genetics 15:146-156 (1997) and Green and Jakdbovits J. Exp. Med. 188:483-495 (1998). The XENOMOUSE® strains are available from Abgenix, Inc. (Fremont, Calif.).

The production of the XENOMOUSE® strains is further discussed and delineated in U.S. patent application Ser. No. 07/466,008, filed Jan. 12, 1990, Ser. No. 07/610,515, filed Nov. 8, 1990, Ser. No. 07/919,297, filed Jul. 24, 1992, Ser. No. 07/922,649, filed Jul. 30, 1992, filed Ser. No. 08/031,801, filed Mar. 15,1993, Ser. No. 08/112,848, filed Aug. 27, 1993, Ser. No. 08/234,145, filed Apr. 28, 1994, Ser. No. 08/376,279, filed Jan. 20, 1995, Ser. No. 08/430,938, Apr. 27, 1995, Ser. No. 08/464,584, filed Jun. 5, 1995, 08/464,582, filed Jun. 5, 1995, Ser. No. 08/463,191, filed Jun. 5, 1995, Ser. No. 08/462,837, filed Jun. 5, 1995, Ser. No. 08/486,853, filed Jun. 5, 1995, Ser. No. 08/486,857, filed Jun. 5, 1995, Ser. No. 08/486,859, filed Jun. 5, 1995, Ser. No. 08/462,513, filed Jun. 5, 1995, Ser. No. 08/724,752, filed Oct. 2, 1996, and Ser. No. 08/759,620, filed Dec. 3, 1996 and U.S. Pat. Nos. 6,162,963, 6,150,584, 6,114,598, 6,075,181, and 5,939,598 and Japanese Patent Nos. 3 068 180 B2, 3 068 506 B2, and 3 068 507 B2. See also Mendez et al. Nature Genetics 15:146-156 (1997) and Green and Jakobovits J. Exp. Med. 188:483-495 (1998). See also European Patent No., EP 0 463 151 B1, grant published Jun. 12, 1996, International Patent Application No., WO 94/02602, published Feb. 3, 1994, International Patent Application No., WO 96/34096, published Oct. 31, 1996, WO 98/24893, published Jun. 11, 1998, WO 00/76310, published Dec. 21, 2000. The disclosures of each of the above-cited patents, applications, and references are hereby incorporated by reference in their entirety.

In an alternative approach, others, including GenPharm International, Inc., have utilized a “minilocus” approach. In the minilocus approach, an exogenous Ig locus is mimicked through the inclusion of pieces (individual genes) from the Ig locus. Thus, one or more V_{H} genes, one or more D_{H} genes, one or more J_{H} genes, a mu constant region, and a second constant region (preferably a gamma constant region) are formed into a construct for insertion into an animal. This approach is described in U.S. Pat. No. 5,545,807 to Surani et al. and U.S. Pat. Nos. 5,545,806, 5,625,825, 5,625,126, 5,633,425, 5,661,016, 5,770,429, 5,789,650, 5,814,318, 5,877,397, 5,874,299, and 6,255,458 each to Lonberg and Kay, U.S. Pat. No. 5,591,669 and 6,023.010 to Krimpenfort and Berns, U.S. Pat. Nos. 5,612,205, 5,721,367, and 5,789,215 to Berns et al., and U.S. Pat. No. 5,643,763 to Choi and Dunn, and GenPharm International U.S. patent application Ser. No. 07/574,748, filed Aug. 29, 1990, Ser. Nos. 07/575,962, filed Aug. 31, 1990, Ser. Nos. 07/810,279, filed Dec. 17, 1991, Ser. Nos. 07/853,408, filed Mar. 18, 1992, Ser. Nos. 07/904,068, filed Jun. 23, 1992, Ser. Nos. 07/990,860, filed Dec. 16, 1992, Ser. Nos. 08/053,131, filed Apr. 26, 1993, Ser. Nos. 08/096,762, filed Jul. 22, 1993, Ser. Nos. 08/155,301, filed Nov. 18, 1993, Ser. Nos. 08/161,739, filed Dec. 3, 1993, Ser. Nos. 08/165,699, filed Dec. 10, 1993, Ser. Nos. 08/209,741, filed Mar. 9, 1994, the disclosures of which are hereby incorporated by reference. See also European Patent No. 0 546 073 B1, International Patent Application Nos. WO 92/03918, WO 92/22645, WO 92/22647, WO 92/22670, WO 93/12227, WO 94/00569, WO 94/25585, WO 96/14436, WO 97/13852, and WO 98/24884 and U.S. Pat. No. 5,981,175, the disclosures of which are hereby incorporated by reference in their entirety. See further Taylor et al., 1992, Chen et al., 1993, Tuaillon et al., 1993, Choi et al., 1993, Lonberg et al., (1994), Taylor et al., (1994), and Tuaillon et al., (1995), Fishwild et al., (1996), the disclosures of which are hereby incorporated by reference in their entirety.

Kirin has also demonstrated the generation of human antibodies from mice in which, through microcell fusion, large pieces of chromosomes, or entire chromosomes, have been introduced. See European Patent Application Nos. 773 288 and 843 961, the disclosures of which are hereby incorporated by reference in their entireties. Additionally, KM™ mice, which are the result of cross-breeding of Kirin's TC mice with Medarex's minilocus (Humab) mice have been generated. These mice possess the HC transchromosome of the Kirin mice and the kappa chain transgene of the Genpharm mice (Ishida et al., Cloning Stem Cells, (2002) 4:91-102).

Human antibodies can also be derived by in vitro methods. Suitable examples include, but are not limited to, phage display (CAT, Morphosys, Dyax, Biosite/Medarex, Xoma, Symphogen, Alexion (formerly Proliferon), Affimed) ribosome display (CAT), yeast display, and the like.

In some embodiments, the antibodies are simply humanized. (See e.g., Winter and Harris Immunol Today 14:43-46 (1993) and Wright et al. Crit. Reviews in Immunol. 12125-168 (1992).) The antibody of interest can be engineered by recombinant DNA techniques to substitute the CH1, CH2, CH3, hinge domains, and/or the framework domain with the corresponding human sequence (see WO 92/02190 and U.S. Pat. Nos. 5,530,101, 5,585,089, 5,693,761, 5,693,792, 5,714,350, and 5,777,085). Also, the use of Ig cDNA for construction of chimeric immunoglobulin genes is known in the art (Liu et al. P.N.A.S. 84:3439 (1987) and J. Immunol. 139:3521 (1987)).

Human anti-mouse antibody (HAMA) responses have also led the industry to prepare chimeric or otherwise humanized antibodies. While chimeric antibodies have a human constant region and a murine variable region, it is expected that certain human anti-chimeric antibody (HACA) responses will be observed, particularly in chronic or multi-dose utilizations of the antibody. Thus, it would be desirable to provide fully human antibodies against multimeric enzymes in order to vitiate concerns and/or effects of HAMA or HACA response.

Preparation of Antibodies

Antibodies, as described herein, were prepared using the XENOMOUSE® technology, as described below. Such mice are capable of producing human immunoglobulin molecules and antibodies and are deficient in the production of murine immunoglobulin molecules and antibodies. Technologies utilized for achieving the same are disclosed in the patents, applications, and references referred to herein. In particular, however, a preferred embodiment of transgenic production of mice and antibodies therefrom is disclosed in U.S. patent application Ser. No. 08/759,620, filed Dec. 3, 1996 and International Patent Application Nos. WO 98/24893, published Jun. 11, 1998 and WO 00/76310, published Dec. 21, 2000, the disclosures of which are hereby incorporated by reference. See also Mendez et al. Nature Genetics 15:146-156 (1997), the disclosure of which is hereby incorporated by reference.

Through use of such technology, fully human monoclonal antibodies to VLA-1 have been produced, as described in detail below. Essentially, XENOMOUSE® lines of mice are immunized with an antigen of interest (e.g., human VLA-1, the alpha-1 subunit of VLA, or fragment thereof), lymphatic cells (such as B-cells) are recovered from mice that expressed antibodies, and the recovered cell lines are fused with a myeloid-type cell line to prepare immortal hybridoma cell lines. These hybridoma cell lines are screened and selected to identify hybridoma cell lines that produced antibodies specific to the antigen of interest. Provided herein are methods for the production of multiple hybridoma cell lines that produce antibodies specific to the desired multimeric enzyme subunit oligomerization domain. Further, provided herein are characterization of the antibodies produced by such cell lines, including nucleotide and amino acid sequence analyses of the heavy and light chains of such antibodies.

Alternatively, instead of being fused to myeloma cells to generate hybridomas, the recovered cells, isolated from immunized XENOMOUSE® lines of mice, are screened further for reactivity against the initial antigen, preferably human VLA-1. Such screening includes ELISA with the desired VLA-1 protein and functional assays. Single B cells secreting antibodies of interest are then isolated using a desired VLA-specific hemolytic plaque assay (Babcook et al., Proc. Natl. Acad. Sci. USA, i93:7843-7848 (1996)). Cells targeted for lysis are preferably sheep red blood cells (SRBCs) coated with the desired VLA-1 antigen. In the presence of a B cell culture secreting the immunoglobulin of interest and complement, the formation of a plaque indicates specific VLA-1-mediated lysis of the target cells.

The single antigen-specific plasma cell in the center of the plaque can be isolated and the genetic information that encodes the specificity of the antibody is isolated from the single plasma cell. Using reverse-transcriptase PCR, the DNA encoding the variable region of the antibody secreted can be cloned. Such cloned DNA can then be further inserted into a suitable expression vector, preferably a vector cassette such as a pcDNA, more preferably such a pcDNA vector containing the constant domains of immunoglobulin heavy and light chain. The generated vector can then be transfected into host cells, preferably CHO cells, and cultured in conventional nutrient media modified as appropriate for inducing promoters, selecting transformants, or amplifying the genes encoding the desired sequences. The genetic material that encodes the specificity of the anti-VLA-1 antibody can be isolated and introduced into a suitable expression vector that is then transfected into host cells.

In general, antibodies produced by the above-mentioned cell lines possessed fully human IgG4 or IgG2 heavy chains with human kappa or lambda light chains. The antibodies possessed high affinities, typically possessing K_{D}s from about 80129 pM through about 25 pM or less.

As mentioned above, anti-VLA-l antibodies can be expressed in cell lines other than hybridoma cell lines. Sequences encoding particular antibodies can be used for transformation of a suitable mammalian host cell, such as a CHO cell. Transformation can be by any known method for introducing polynucleotides into a host cell, including, for example packaging the polynucleotide in a virus (or into a viral vector) and transducing a host cell with the virus (or vector) or by transfection procedures known in the art, as exemplified by U.S. Pat. Nos. 4,399,216, 4,912,040, 4,740,461, and 4,959,455 (which patents are hereby incorporated herein by reference). The transformation procedure used depends upon the host to be transformed. Methods for introducing heterologous polynucleotides into mammalian cells are well known in the art and include dextran-mediated transfection, calcium phosphate precipitation, polybrene mediated transfection, protoplast fusion, electroporation, encapsulation of the polynucleotide(s) in liposomes, and direct microinjection of the DNA into nuclei.

Mammalian cell lines available as hosts for expression are well known in the art and include many immortalized cell lines available from the American Type Culture Collection (ATCC), including but not limited to Chinese hamster ovary (CHO) cells, HeLa cells, baby hamster kidney (BHK) cells, monkey kidney cells (COS), human hepatocellular carcinoma cells (e.g., Hep G2), and a number of other cell lines. Cell lines of particular preference are selected through determining which cell lines have high expression levels and produce antibodies with VLA-1 binding properties.

As will be appreciated by one of skill in the art, one can raise antibodies to various fragments or parts of the VLA-l protein. In alternative embodiments, the antigen used can vary dramatically. While antibodies that bind directly to the alpha 1 subunit may possess the ability to prevent VLA-1 activity, antibodies that bind to other sections of the VLA-1 complex can also be effective while still be relatively specific for the VLA-1 complex and for activities related to the VLA-1 complex. For example, the antibody can bind to an interface between the alpha 1 and beta 1 subunit. Alternatively, the antibody can bind to the beta 1 subunit, but only to those beta 1 subunits that are in the structural conformation that the beta 1 subunit is in when the beta 1 and alpha 1 subunits are complexed in a heterodimer. In some embodiments, the antibodies do more than simply prevent VLA-1 from binding to another molecule (e.g., collagen). For example, the antibodies can dissociate the alpha 1 and beta 1 subunits. Thus, in some embodiments, the VLA-1 complex is formed and dissociates upon binding with the antibody. One of skill in the art, in light of the present teachings, will be able to select the appropriate antigen to use to create the antibody and then to screen through those antibodies to select the appropriately functional antibody.

Antibody Sequences

The heavy chain and light chain variable region nucleotide and amino acid sequences of representative human anti-VLA-1 antibodies are provided in the sequence listing, the contents of which are summarized in Table 1 below. The “mAb ID No:” is a reference for each of the antibodies. Each number has two to three registers, e.g., “XX.YY” or “XX.YY.ZZ” The “YY” numbers can be denoted as either single digits e.g., “6,” or as dual digits, e.g., “06” for the same antibody. When present, the “ZZ” digits denote different selections of the same antibody clone.

TABLE 1

| mAb | | SEQ ID |
|---|---|---|
| ID No.: | Sequence | NO: |
| 1.7 | Heavy Chain DNA | 1 |
| | Heavy Chain Protein | 2 |
| | Light Chain DNA | 3 |
| | Light Chain Protein | 4 |
| 2.10 | Heavy Chain DNA | 5 |
| | Heavy Chain Protein | 6 |
| | Light Chain DNA | 7 |
| | Light Chain Protein | 8 |
| 2.11 | Heavy Chain DNA | 9 |
| | Heavy Chain Protein | 10 |
| | Light Chain DNA | 11 |
| | Light Chain Protein | 12 |
| 2.12 | Heavy Chain DNA | 13 |
| | Heavy Chain Protein | 14 |
| | Light Chain DNA | 15 |
| | Light Chain Protein | 16 |
| 2.14.1 | Heavy Chain DNA | 17 |
| | Heavy Chain Protein | 18 |
| | Light Chain DNA | 19 |
| | Light Chain Protein | 20 |
| 2.14.3 | Heavy Chain DNA | 21 |
| | Heavy Chain Protein | 22 |
| | Light Chain DNA | 23 |
| | Light Chain Protein | 24 |
| 2.17 | Heavy Chain DNA | 25 |
| | Heavy Chain Protein | 26 |
| | Light Chain DNA | 27 |
| | Light Chain Protein | 28 |
| 2.19 | Heavy Chain DNA | 29 |
| | Heavy Chain Protein | 30 |
| | Light Chain DNA | 31 |
| | Light Chain Protein | 32 |
| 2.1 | Heavy Chain DNA | 33 |
| | Heavy Chain Protein | 34 |
| | Light Chain DNA | 35 |
| | Light Chain Protein | 36 |
| 2.25 | Heavy Chain DNA | 37 |
| | Heavy Chain Protein | 38 |
| | Light Chain DNA | 39 |
| | Light Chain Protein | 40 |
| 2.26 | Heavy Chain DNA | 41 |
| | Heavy Chain Protein | 42 |
| | Light Chain DNA | 43 |
| | Light Chain Protein | 44 |
| 2.2 | Heavy Chain DNA | 45 |
| | Heavy Chain Protein | 46 |
| | Light Chain DNA | 47 |
| | Light Chain Protein | 48 |
| 2.3 | Heavy Chain DNA | 49 |
| | Heavy Chain Protein | 50 |
| | Light Chain DNA | 51 |
| | Light Chain Protein | 52 |
| 2.7 | Heavy Chain DNA | 53 |
| | Heavy Chain Protein | 54 |
| | Light Chain DNA | 55 |
| | Light Chain Protein | 56 |
| 3.11 | Heavy Chain DNA | 57 |
| | Heavy Chain Protein | 58 |
| | Light Chain DNA | 59 |
| | Light Chain Protein | 60 |
| 3.12 | Heavy Chain DNA | 61 |
| | Heavy Chain Protein | 62 |
| | Light Chain DNA | 63 |
| | Light Chain Protein | 64 |
| 3.14 | Heavy Chain DNA | 65 |
| | Heavy Chain Protein | 66 |
| | Light Chain DNA | 67 |
| | Light Chain Protein | 68 |
| 3.17 | Heavy Chain DNA | 69 |
| | Heavy Chain Protein | 70 |
| | Light Chain DNA | 71 |
| | Light Chain Protein | 72 |
| 3.18 | Heavy Chain DNA | 73 |
| | Heavy Chain Protein | 74 |
| | Light Chain DNA | 75 |
| | Light Chain Protein | 76 |
| 3.19 | Heavy Chain DNA | 77 |
| | Heavy Chain Protein | 78 |
| | Light Chain DNA | 79 |
| | Light Chain Protein | 80 |
| 3.1 | Heavy Chain DNA | 81 |
| | Heavy Chain Protein | 82 |
| | Light Chain DNA | 83 |
| | Light Chain Protein | 84 |
| 3.20 | Heavy Chain DNA | 85 |
| | Heavy Chain Protein | 86 |
| | Light Chain DNA | 87 |
| | Light Chain Protein | 88 |
| 3.21 | Heavy Chain DNA | 89 |
| | Heavy Chain Protein | 90 |
| | Light Chain DNA | 91 |
| | Light Chain Protein | 92 |
| 3.24 | Heavy Chain DNA | 93 |
| | Heavy Chain Protein | 94 |
| | Light Chain DNA | 95 |
| | Light Chain Protein | 96 |
| 3.2 | Heavy Chain DNA | 97 |
| | Heavy Chain Protein | 98 |
| | Light Chain DNA | 99 |
| | Light Chain Protein | 100 |
| 3.31 | Heavy Chain DNA | 101 |
| | Heavy Chain Protein | 102 |
| | Light Chain DNA | 103 |
| | Light Chain Protein | 104 |
| 3.32 | Heavy Chain DNA | 105 |
| | Heavy Chain Protein | 106 |
| | Light Chain DNA | 107 |
| | Light Chain Protein | 108 |
| 3.33 | Heavy Chain DNA | 109 |
| | Heavy Chain Protein | 110 |
| | Light Chain DNA | 111 |
| | Light Chain Protein | 112 |
| 3.34 | Heavy Chain DNA | 113 |
| | Heavy Chain Protein | 114 |
| | Light Chain DNA | 115 |
| | Light Chain Protein | 116 |
| 3.36 | Heavy Chain DNA | 117 |
| | Heavy Chain Protein | 118 |
| | Light Chain DNA | 119 |
| | Light Chain Protein | 120 |
| 3.38 | Heavy Chain DNA | 121 |
| | Heavy Chain Protein | 122 |
| | Light Chain DNA | 123 |
| | Light Chain Protein | 124 |
| 3.3 | Heavy Chain DNA | 125 |
| | Heavy Chain Protein | 126 |
| | Light Chain DNA | 127 |
| | Light Chain Protein | 128 |
| 3.40 | Heavy Chain DNA | 129 |
| | Heavy Chain Protein | 130 |
| | Light Chain DNA | 131 |
| | Light Chain Protein | 132 |
| 3.4 | Heavy Chain DNA | 133 |
| | Heavy Chain Protein | 134 |
| | Light Chain DNA | 135 |
| | Light Chain Protein | 136 |
| 3.5 | Heavy Chain DNA | 137 |
| | Heavy Chain Protein | 138 |
| | Light Chain DNA | 139 |
| | Light Chain Protein | 140 |
| 3.7 | Heavy Chain DNA | 141 |
| | Heavy Chain Protein | 42 |
| | Light Chain DNA | 143 |
| | Light Chain Protein | 144 |
| 3.8 | Heavy Chain DNA | 145 |
| | Heavy Chain Protein | 146 |
| | Light Chain DNA | 147 |
| | Light Chain Protein | 148 |
| 3.9 | Heavy Chain DNA | 149 |
| | Heavy Chain Protein | 150 |
| | Light Chain DNA | 151 |
| | Light Chain Protein | 152 |
| 4.13 | Heavy Chain DNA | 153 |
| | Heavy Chain Protein | 154 |
| | Light Chain DNA | 155 |
| | Light Chain Protein | 156 |
| 4.20 | Heavy Chain DNA | 157 |
| | Heavy Chain Protein | 158 |
| | Light Chain DNA | 159 |
| | Light Chain Protein | 160 |
| 4.2 | Heavy Chain DNA | 161 |
| | Heavy Chain Protein | 162 |
| | Light Chain DNA | 163 |
| | Light Chain Protein | 164 |
| 4.3 | Heavy Chain DNA | 165 |
| | Heavy Chain Protein | 166 |
| | Light Chain DNA | 167 |
| | Light Chain Protein | 168 |
| 4.5 | Heavy Chain DNA | 169 |
| | Heavy Chain Protein | 170 |
| | Light Chain DNA | 171 |
| | Light Chain Protein | 172 |
| 5.3 | Heavy Chain DNA | 173 |
| | Heavy Chain Protein | 174 |
| | Light Chain DNA | 175 |
| | Light Chain Protein | 176 |
| 5.4 | Heavy Chain DNA | 177 |
| | Heavy Chain Protein | 178 |
| | Light Chain DNA | 179 |
| | Light Chain Protein | 180 |
| 5.5 | Heavy Chain DNA | 181 |
| | Heavy Chain Protein | 182 |
| | Light Chain DNA | 183 |
| | Light Chain Protein | 184 |
| 6.15 | Heavy Chain DNA | 185 |
| | Heavy Chain Protein | 186 |
| | Light Chain DNA | 187 |
| | Light Chain Protein | 188 |
| 6.17 | Heavy Chain DNA | 189 |
| | Heavy Chain Protein | 190 |
| | Light Chain DNA | 191 |
| | Light Chain Protein | 192 |
| 6.18 | Heavy Chain DNA | 193 |
| | Heavy Chain Protein | 194 |
| | Light Chain DNA | 195 |
| | Light Chain Protein | 196 |
| 6.20 | Heavy Chain DNA | 197 |
| | Heavy Chain Protein | 198 |
| | Light Chain DNA | 199 |
| | Light Chain Protein | 200 |
| 6.3 | Heavy Chain DNA | 201 |
| | Heavy Chain Protein | 202 |
| | Light Chain DNA | 203 |
| | Light Chain Protein | 204 |
| 6.44 | Heavy Chain DNA | 205 |
| | Heavy Chain Protein | 206 |
| | Light Chain DNA | 207 |
| | Light Chain Protein | 208 |
| 6.48 | Heavy Chain DNA | 209 |
| | Heavy Chain Protein | 210 |
| | Light Chain DNA | 211 |
| | Light Chain Protein | 212 |
| 6.58 | Heavy Chain DNA | 213 |
| | Heavy Chain Protein | 214 |
| | Light Chain DNA | 215 |
| | Light Chain Protein | 216 |
| 6.8 | Heavy Chain DNA | 217 |
| | Heavy Chain Protein | 218 |
| | Light Chain DNA | 219 |
| | Light Chain Protein | 220 |
| 7.10 | Heavy Chain DNA | 221 |
| | Heavy Chain Protein | 222 |
| | Light Chain DNA | 223 |
| | Light Chain Protein | 224 |
| 7.11 | Heavy Chain DNA | 225 |
| | Heavy Chain Protein | 226 |
| | Light Chain DNA | 227 |
| | Light Chain Protein | 228 |
| 7.13 | Heavy Chain DNA | 229 |
| | Heavy Chain Protein | 230 |
| | Light Chain DNA | 231 |
| | Light Chain Protein | 232 |
| 7.14 | Heavy Chain DNA | 233 |
| | Heavy Chain Protein | 234 |
| | Light Chain DNA | 235 |
| | Light Chain Protein | 236 |
| 7.15 | Heavy Chain DNA | 237 |
| | Heavy Chain Protein | 238 |
| | Light Chain DNA | 239 |
| | Light Chain Protein | 240 |
| 7.16 | Heavy Chain DNA | 241 |
| | Heavy Chain Protein | 242 |
| | Light Chain DNA | 243 |
| | Light Chain Protein | 244 |
| 7.18 | Heavy Chain DNA | 245 |
| | Heavy Chain Protein | 246 |
| | Light Chain DNA | 247 |
| | Light Chain Protein | 248 |
| 7.22 | Heavy Chain DNA | 249 |
| | Heavy Chain Protein | 250 |
| | Light Chain DNA | 251 |
| | Light Chain Protein | 252 |
| 7.24 | Heavy Chain DNA | 253 |
| | Heavy Chain Protein | 254 |
| | Light Chain DNA | 255 |
| | Light Chain Protein | 256 |
| 7.34 | Heavy Chain DNA | 257 |
| | Heavy Chain Protein | 258 |
| | Light Chain DNA | 259 |
| | Light Chain Protein | 260 |
| 7.35 | Heavy Chain DNA | 261 |
| | Heavy Chain Protein | 262 |
| | Light Chain DNA | 263 |
| | Light Chain Protein | 264 |
| 7.36 | Heavy Chain DNA | 265 |
| | Heavy Chain Protein | 266 |
| | Light Chain DNA | 267 |
| | Light Chain Protein | 268 |
| 7.37 | Heavy Chain DNA | 269 |
| | Heavy Chain Protein | 270 |
| | Light Chain DNA | 271 |
| | Light Chain Protein | 272 |
| 7.38 | Heavy Chain DNA | 273 |
| | Heavy Chain Protein | 274 |
| | Light Chain DNA | 275 |
| | Light Chain Protein | 276 |
| 7.3 | Heavy Chain DNA | 277 |
| | Heavy Chain Protein | 278 |
| | Light Chain DNA | 279 |
| | Light Chain Protein | 280 |
| 7.40 | Heavy Chain DNA | 281 |
| | Heavy Chain Protein | 282 |
| | Light Chain DNA | 283 |
| | Light Chain Protein | 284 |
| 7.41 | Heavy Chain DNA | 285 |
| | Heavy Chain Protein | 286 |
| | Light Chain DNA | 287 |
| | Light Chain Protein | 288 |
| 7.46 | Heavy Chain DNA | 289 |
| | Heavy Chain Protein | 290 |
| | Light Chain DNA | 291 |
| | Light Chain Protein | 292 |
| 7.47 | Heavy Chain DNA | 293 |
| | Heavy Chain Protein | 294 |
| | Light Chain DNA | 295 |
| | Light Chain Protein | 296 |
| 7.4 | Heavy Chain DNA | 297 |
| | Heavy Chain Protein | 298 |
| | Light Chain DNA | 299 |
| | Light Chain Protein | 300 |
| 7.52 | Heavy Chain DNA | 301 |
| | Heavy Chain Protein | 302 |
| | Light Chain DNA | 303 |
| | Light Chain Protein | 304 |
| 7.57 | Heavy Chain DNA | 305 |
| | Heavy Chain Protein | 306 |
| | Light Chain DNA | 307 |
| | Light Chain Protein | 308 |
| 7.60 | Heavy Chain DNA | 309 |
| | Heavy Chain Protein | 310 |
| | Light Chain DNA | 311 |
| | Light Chain Protein | 312 |
| 7.62 | Heavy Chain DNA | 313 |
| | Heavy Chain Protein | 314 |
| | Light Chain DNA | 315 |
| | Light Chain Protein | 316 |
| 7.63 | Heavy Chain DNA | 317 |
| | Heavy Chain Protein | 318 |
| | Light Chain DNA | 319 |
| | Light Chain Protein | 320 |
| 7.66 | Heavy Chain DNA | 321 |
| | Heavy Chain Protein | 322 |
| | Light Chain DNA | 323 |
| | Light Chain Protein | 324 |
| 7.69 | Heavy Chain DNA | 325 |
| | Heavy Chain Protein | 326 |
| | Light Chain DNA | 327 |
| | Light Chain Protein | 328 |
| 7.71 | Heavy Chain DNA | 329 |
| | Heavy Chain Protein | 330 |
| | Light Chain DNA | 331 |
| | Light Chain Protein | 332 |
| 7.73 | Heavy Chain DNA | 333 |
| | Heavy Chain Protein | 334 |
| | Light Chain DNA | 335 |
| | Light Chain Protein | 336 |
| 7.7 | Heavy Chain DNA | 337 |
| | Heavy Chain Protein | 338 |
| | Light Chain DNA | 339 |
| | Light Chain Protein | 340 |
| 7.8 | Heavy Chain DNA | 341 |
| | Heavy Chain Protein | 342 |
| | Light Chain DNA | 343 |
| | Light Chain Protein | 344 |
| 7.9 | Heavy Chain DNA | 345 |
| | Heavy Chain Protein | 346 |
| | Light Chain DNA | 347 |
| | Light Chain Protein | 348 |
| 8.5 | Heavy Chain DNA | 349 |
| | Heavy Chain Protein | 350 |
| | Light Chain DNA | 351 |
| | Light Chain Protein | 352 |
| 8.7 | Heavy Chain DNA | 353 |
| | Heavy Chain Protein | 354 |
| | Light Chain DNA | 355 |
| | Light Chain Protein | 356 |
| 9.11 | Heavy Chain DNA | 357 |
| | Heavy Chain Protein | 358 |
| | Light Chain DNA | 359 |
| | Light Chain Protein | 360 |
| 9.9 | Heavy Chain DNA | 361 |
| | Heavy Chain Protein | 362 |
| | Light Chain DNA | 363 |
| | Light Chain Protein | 364 |
| 10.1 | Heavy Chain DNA | 365 |
| | Heavy Chain Protein | 366 |
| | Light Chain DNA | 367 |
| | Light Chain Protein | 368 |
| 10.2 | Heavy Chain DNA | 369 |
| | Heavy Chain Protein | 370 |
| | Light Chain DNA | 371 |
| | Light Chain Protein | 372 |
| 10.3 | Heavy Chain DNA | 373 |
| | Heavy Chain Protein | 374 |
| | Light Chain DNA | 375 |
| | Light Chain Protein | 376 |
| 10.4 | Heavy Chain DNA | 377 |
| | Heavy Chain Protein | 378 |
| | Light Chain DNA | 379 |
| | Light Chain Protein | 380 |
| 10.5 | Heavy Chain DNA | 381 |
| | Heavy Chain Protein | 382 |
| | Light Chain DNA | 383 |
| | Light Chain Protein | 384 |
| 10.6 | Heavy Chain DNA | 385 |
| | Heavy Chain Protein | 386 |
| | Light Chain DNA | 387 |
| | Light Chain Protein | 388 |
| 10.9 | Heavy Chain DNA | 389 |
| | Heavy Chain Protein | 390 |
| | Light Chain DNA | 391 |
| | Light Chain Protein | 392 |
| 10.10 | Heavy Chain DNA | 393 |
| | Heavy Chain Protein | 394 |
| | Light Chain DNA | 395 |
| | Light Chain Protein | 396 |
| 10.11 | Heavy Chain DNA | 397 |
| | Heavy Chain Protein | 398 |
| | Light Chain DNA | 399 |
| | Light Chain Protein | 400 |
| 10.13 | Heavy Chain DNA | 401 |
| | Heavy Chain Protein | 402 |
| | Light Chain DNA | 403 |
| | Light Chain Protein | 404 |
| 10.15 | Heavy Chain DNA | 405 |
| | Heavy Chain Protein | 406 |
| | Light Chain DNA | 407 |
| | Light Chain Protein | 408 |
| 10.18 | Heavy Chain DNA | 409 |
| | Heavy Chain Protein | 410 |
| | Light Chain DNA | 411 |
| | Light Chain Protein | 412 |

Antibody Therapeutics

Anti-VLA-1 antibodies will have therapeutic effects in treating symptoms and conditions related to VLA-1 activity. VLA-1 has been implicated in a wide variety of diseases and disorders, including chronic inflammation and fibrosis disorders in which VLA-1 activity level is elevated in one or more tissues as compared to a normal subject. Examples of relevant VLA-1 mediated disorders are inflammatory disorders, such as immune-mediated inflammatory disorders (IMIDs), which are inflammatory conditions caused and sustained by an antigen-specific, pathological immune response. Among these disorders are various types of arthritis, such as rheumatoid arthritis, as well as allergic diseases, such as asthma, hay fever, and urticaria; different types of connective tissue disorders; inflammatory bowel disease (e.g., Crohn's disease and ulcerative colitis); insulin-dependent diabetes; uveitis; retinitis; graft rejection; and graft-versus host-disease. VLA-1 related disorders can also include tissue inflammation in infectious, ischemic, hemorrhagic, and traumatic conditions, e.g., fasciitis, stroke, infarction of the myocardium and other organs (e.g., lung and intestine), ARDS; hepatitis, (e.g., infectious and non-infectious, acute and chronic); acute and chronic pancreatitis; reperfusion injuries; radiation injuries; vascular restenosis of different types (e.g., coronary restenosis). Also included are osteoarthritis, osteoporosis, atherosclerosis, organ fibrosis, and neurodegenerative disorders such as Alzheimer's disease and multiple sclerosis. VLA-1 related disorders can include cancer, blood malignancies, e.g., leukemias and multiple myelomas; the development of a number of solid tumors, tumor growth, and metastatic spreading, or some combination thereof. In some embodiments, the VLA-1 related or mediated disorder is selected from the group of skin related conditions, allergic rhinitis, respiratory distress syndrome, bronchitis, tendinitis, bursitis, fever, migraine headaches, gastrointestinal conditions, periarteritis nodosa, thyroiditis, aplastic anemia, Hodgkin's Disease, rheumatic fever, autoimmune diseases, sarcoidosis, nephrotic syndrome, renal failure, Behcet's Syndrome, polymyositis, gingivitis, hypersensitivity, conjunctivitis, swelling occurring after injury, myocardial ischemia, endotoxin shock syndrome, renal scarring, and eosinophilic inflammation, or some combination thereof. A “VLA-1 related” or mediated disorder is one in which there is an excess of VLA-1 or VLA-1 activity.

In addition, the anti-VLA-1 antibodies can be useful as diagnostics for the disease states described above, including, e.g., asthma and inflammation related diseases. The antibodies can be added to a sample from a patient and excessive binding of the antibody to a sample in the patient can indicate an excessive level of VLA-1; thus, indicating the presence of one of the above disease states. The amount, distribution, and localization of the antibodies or the sample can further be used to identify which of the above disease states are involved, as well as additional assays known to one of skill in the art. These antibodies can be observed either directly, e.g., through fluorescent probes attached to the antibodies, or indirectly, e.g., through additional antibodies that bind to the antibodies above. In some embodiments, the level of VLA-1 is compared to a healthy or control amount of VLA-1, where a significant difference is indicative of a VLA-1 related disease. In some embodiments, the antibodies described above are configured to bind to VLA-1 proteins that are not bound to a ligand. In other embodiments, the VLA-1 antibody is configured to bind to VLA-1 proteins that are also bound to a ligand, e.g., collagen I, IV, and laminin. In some embodiments the antibody will selectively bind to the bound or unbound state of the VLA-1 protein, but not significantly to the other state of the VLA-1. protein.

If desired, the isotype of an anti-VLA-1 antibody can be switched, for example to take advantage of a biological property of a different isotype. For example, in some circumstances it may be desirable in connection with the generation of antibodies as therapeutic antibodies against VLA-1 that the antibodies be capable of fixing complement and participating in complement-dependent cytotoxicity (CDC). There are a number of isotypes of antibodies that are capable of the same, including, without limitation, the following: murine IgM, murine IgG2a, murine IgG2b, murine IgG3, human IgM, human IgG1, and human IgG3. It will be appreciated that antibodies that are generated need not initially possess such an isotype but, rather, the antibody as generated can possess any isotype and the antibody can be isotype switched thereafter using conventional techniques that are well known in the art. Such techniques include the use of direct recombinant techniques (see e.g., U.S. Patent No. 4,816,397), cell-cell fusion techniques (see e.g., U.S. Pat. Nos. 5,916,771 and 6,207,418), among others.

By way of example, the anti-VLA-1 antibodies discussed herein are fully human antibodies. If an antibody possessed desired binding to VLA-1, it could be readily isotype switched to generate a human IgM, human IgG1, or human IgG3 isotype, while still possessing the same variable region (which defines the antibody's specificity and some of its affinity). Such molecule would then be capable of fixing complement and participating in CDC.

In the cell-cell fusion technique, a myeloma or other cell line is prepared that possesses a heavy chain with any desired isotype and another myeloma or other cell line is prepared that possesses the light chain. Such cells can, thereafter, be fused and a cell line expressing an intact antibody can be isolated. (See, e.g., U.S. Pat. No. 6,677,138, incorporated by reference in its entirety).

Accordingly, as antibody candidates are generated that meet desired “structural” attributes as discussed above, they can generally be provided with at least certain of the desired “functional” attributes through isotype switching.

Biologically active antibodies that bind VLA-l are preferably used in a sterile pharmaceutical preparation or formulation to reduce the activity of VLA-1. Anti-VLA-1 antibodies preferably possess adequate affinity to potently suppress VLA-1 activity to within the target therapeutic range. The suppression preferably results from the ability of the antibody to interfere with the binding of VLA-1 to a matrix ligand, such as collagen (e.g., collagen IV, collagen I, and laminin).

When used for in vivo administration, the antibody formulation is preferably sterile. This is readily accomplished by any method know in the art, for example by filtration through sterile filtration membranes. The antibody ordinarily will be stored in lyophilized form or in solution. Sterile filtration may be performed prior to or following lyophilization and reconstitution.

Therapeutic antibody compositions generally are placed into a container having a sterile access port, for example, an intravenous solution bag or vial having an adapter that allows retrieval of the formulation, such as a stopper pierceable by a hypodermic injection needle.

The route of antibody administration is in accord with known methods, e.g., injection or infusion by intravenous, intraperitoneal, intracerebral, intramuscular, intraocular, intraarterial, intrathecal, inhalation or intralesional routes, or by sustained release systems as noted below. In some situations the antibody is preferably administered by infusion or by bolus injection. In other situations a therapeutic composition comprising the antibody can be administered through the nose or lung, preferably as a liquid or powder aerosol (lyophilized). The composition may also be administered intravenously, parenterally or subcutaneously as desired. When administered systemically, the therapeutic composition should be sterile, pyrogen-free and in a parenterally acceptable solution having due regard for pH, isotonicity, and stability. These conditions are known to those skilled in the art. The antibody is preferably administered continuously by infusion or by bolus injection.

Antibodies, as described herein, can be prepared in a mixture with a pharmaceutically acceptable carrier. This therapeutic composition can be administered intravenously or through the nose or lung, preferably as a liquid or powder aerosol (lyophilized). The composition may also be administered parenterally or subcutaneously as desired. When administered systemically, the therapeutic composition should be sterile, pyrogen-free and in a parenterally acceptable solution having due regard for pH, isotonicity, and stability. These conditions are known to those skilled in the art. Briefly, dosage formulations of the compounds described herein are prepared for storage or administration by mixing the compound having the desired degree of purity with physiologically acceptable carriers, excipients, or stabilizers. Such materials are non-toxic to the recipients at the dosages and concentrations employed, and include buffers such as TRIS HCl, phosphate, citrate, acetate and other organic acid salts; antioxidants such as ascorbic acid; low molecular weight (less than about ten residues) peptides such as polyarginine, proteins, such as serum albumin, gelatin, or immunoglobulins; hydrophilic polymers such as polyvinylpyrrolidinone; amino acids such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium and/or nonionic surfactants such as TWEEN, PLURONICS or polyethyleneglycol.

Sterile compositions for injection can be formulated according to conventional pharmaceutical practice as described in Remington: The Science and Practice of Pharmacy (20^{th} ed, Lippincott Williams & Wilkens Publishers (2003)). For example, dissolution or suspension of the active compound in a vehicle such as water or naturally occurring vegetable oil like sesame, peanut, or cottonseed oil or a synthetic fatty vehicle like ethyl oleate or the like may be desired. Buffers, preservatives, antioxidants, and the like can be incorporated according to accepted pharmaceutical practice.

Suitable examples of sustained-release preparations include semipermeable matrices of solid hydrophobic polymers containing the polypeptide, which matrices are in the form of shaped articles, films or microcapsules. Examples of sustained-release matrices include polyesters, hydrogels (e.g., poly(2-hydroxyethyl-methacrylate) as described by Langer et al., J. Biomed Mater. Res., (1981) 15:167-277 and Langer, Chem. Tech., (1982) 12:98-105, or poly(vinylalcohol)), polylactides (U.S. Pat. No. 3,773,919, EP 58,481), copolymers of L-glutamic acid and gamma ethyl-L-glutamate (Sidman et al., Biopolymers, (1983) 22:547-556), non-degradable ethylene-vinyl acetate (Langer et al., supra), degradable lactic acid-glycolic acid copolymers such as the LUPRON Depot™ (injectable microspheres composed of lactic acid-glycolic acid copolymer and leuprolide acetate), and poly-D-(−)-3-hydroxybutyric acid (EP. 133,988).

While polymers such as ethylene-vinyl acetate and lactic acid-glycolic acid enable release of molecules for over 100 days, certain hydrogels release proteins for shorter time periods. When encapsulated proteins remain in the body for a long time, they may denature or aggregate as a result of exposure to moisture at 37° C., resulting in a loss of biological activity and possible changes in immunogenicity. Rational strategies can be devised for protein stabilization depending on the mechanism involved. For example, if the aggregation mechanism is discovered to be intermolecular S-S bond formation through disulfide interchange, stabilization may be achieved by modifying sulfhydryl residues, lyophilizing from acidic solutions, controlling moisture content, using appropriate additives, and developing specific polymer matrix compositions.

Sustained-released compositions also include preparations of crystals of the antibody suspended in suitable formulations capable of maintaining crystals in suspension. These preparations when injected subcutaneously or intraperitonealy can produce a sustained release effect. Other compositions also include liposomally entrapped antibodies. Liposomes containing such antibodies are prepared by methods known per se: U.S. Pat. No. DE 3,218,121; Epstein et al., Proc. Natl. Acad. Sci. USA, (1985) 82:3688-3692; Hwang et al., Proc. Natl. Acad. Sci. USA, (1980) 77:4030-4034; EP 52,322; EP 36,676; EP 88,046; EP 143,949; 142,641; Japanese patent application 83-118008; U.S. Pat. Nos. 4,485,045 and 4,544,545; and EP 102,324.

The dosage of the antibody formulation for a given patient will be determined by the attending physician taking into consideration various factors known to modify the action of drugs including severity and type of disease, body weight, sex, diet, time and route of administration, other medications and other relevant clinical factors. Therapeutically effective dosages may be determined by either in vitro or in vivo methods.

An effective amount of the antibodies, described herein, to be employed therapeutically will depend, for example, upon the therapeutic objectives, the route of administration, and the condition of the patient. Accordingly, it is preferred for the therapist to titer the dosage and modify the route of administration as required to obtain the optimal therapeutic effect. A typical daily dosage might range from about 0.001 mg/kg to up to 100 mg/kg or more, depending on the factors mentioned above. Typically, the clinician will administer the therapeutic antibody until a dosage is reached that achieves the desired effect. The progress of this therapy is easily monitored by conventional assays.

It is expected that the antibodies described herein will have therapeutic effect in treatment of symptoms and conditions resulting from or related to the activity of VLA-1. The above antibodies and additional compositions can also be included in medicaments for the treatment of VLA-1 related or mediated disorders. Similarly, such compounds can be used in the preparation of a medicament for the treatment of a VLA-1 mediated or related disorder.

Design and Generation of Other Therapeutics

In accordance with the present invention and based on the activity of the antibodies that are produced and characterized herein with respect to VLA-1, the design of other therapeutic modalities is facilitated and disclosed to one of skill in the art. Such modalities include, without limitation, advanced antibody therapeutics, such as bispecific antibodies, immunotoxins, and radiolabeled therapeutics, generation of peptide therapeutics, gene therapies, particularly intrabodies, antisense therapeutics, and small molecules.

In connection with the generation of advanced antibody therapeutics, where complement fixation is a desirable attribute, it may be possible to sidestep the dependence on complement for cell killing through the use of bispecifics, immunotoxins, or radiolabels, for example.

For example, bispecific antibodies can be generated that comprise (i) two antibodies, one with a specificity to VLA-1 and another to a second molecule, that are conjugated together, (ii) a single antibody that has one chain specific to VLA-1 and a second chain specific to a second molecule, or (iii) a single chain antibody that has specificity to both VLA-1 and the other molecule. Such bispecific antibodies can be generated using techniques that are well known; for example, in connection with (i) and (ii) see e.g., Fanger et al. Immunol Methods 4:72-81 (1994) and Wright and Harris, supra. and in connection with (iii) see e.g., Traunecker et al. Int. J. Cancer (Suppl.) 7:51-52 (1992). In each case, the second specificity can be made as desired. For example, the second specificity can be made to the heavy chain activation receptors, including, without limitation, CD16 or CD64 (see e.g., Deo et al. 18:127 (1997)) or CD89 (see e.g., Valerius et al. Blood 90:4485-4492 (1997)).

Antibodies can also be modified to act as immunotoxins utilizing techniques that are well known in the art. See e.g., Vitetta Immunol Today 14:252 (1993). See also U.S. Pat. No. 5,194,594. In connection with the preparation of radiolabeled antibodies, such modified antibodies can also be readily prepared utilizing techniques that are well known in the art. See e.g., Junghans et al. in Cancer Chemotherapy and Biotherapy 655-686 (2d edition, Chafner and Longo, eds., Lippincott Raven (1996)). See also U.S. Pat. Nos. 4,681,581, 4,735,210, 5,101,827, 5,102,990 (RE 35,500), U.S. Pat. No. 5,648,471, and 5,697,902. Each of immunotoxins and radiolabeled molecules would be likely to kill cells expressing the desired multimeric enzyme subunit oligomerization domain. In some embodiments, a pharmaceutical composition comprising an effective amount of the antibody in association with a pharmaceutically acceptable carrier or diluent is provided. In some embodiments, an anti-VLA-1 antibody is linked to a radioisotope or a toxin. Preferably, such antibodies may be used for the treatment of disorders, such chronic inflammation and fibrosis disorders in which the VLA-1 activity level is elevated in one or more tissues as compared to a normal subject. Examples of such disorders are inflammatory disorders, such as immune-mediated inflammatory disorders (IMIDs), which are inflammatory conditions caused and sustained by an antigen-specific, pathological immune response. Among these disorders are various types of arthritis, such as rheumatoid arthritis, as well as allergic diseases, such as asthma, hay fever, and urticaria; different types of connective tissue disorders; inflammatory bowel disease (e.g., Crohn's disease and ulcerative colitis); insulin-dependent diabetes; uveitis; retinitis; graft rejection; and graft-versus host-disease. VLA-1 related disorders can also include tissue inflammation in infectious, ischemic, hemorrhagic, and traumatic conditions, e.g., fasciitis, stroke, infarction of the myocardium and other organs (e.g., lung and intestine), ARDS; hepatitis, (e.g., infectious and non-infectious, acute and chronic); acute and chronic pancreatitis; reperfusion injuries; radiation injuries; vascular restenosis of different types (e.g., coronary restenosis). Also included are osteoarthritis, osteoporosis, atherosclerosis, organ fibrosis, and neurodegenerative disorders such as Alzheimer's disease and multiple sclerosis. VLA-1 related disorders can include cancer, blood malignancies, e.g., leukemias and multiple myelomas; the development of a number of solid tumors, tumor growth, and metastatic spreading, or some combination thereof. In some embodiments, the VLA-1 related or mediated disorders is selected from the group of psoriasis, skin related conditions, allergic rhinitis, respiratory distress syndrome, bronchitis, tendinitis, bursitis, fever, migraine headaches, gastrointestinal conditions, periarteritis nodosa, thyroiditis, aplastic anemia, Hodgkin's Disease, rheumatic fever, autoimmune diseases, sarcoidosis, nephrotic syndrome, renal failure, Behcet's Syndrome, polymyositis, gingivitis, hypersensitivity, conjunctivitis, swelling occurring after injury, myocardial ischemia, endotoxin shock syndrome, renal scarring, and eosinophilic inflammation, and some combination thereof. The above compositions can also be included in medicaments for the treatment of VLA-1 related or mediated disorders. Similarly, such compounds can be used in the preparation of a medicament for the treatment of a VLA-1 mediated disorder.

In other embodiments, the antibodies disclosed herein provide an assay kit and/or method for the detection of VLA-1 in mammalian tissues or cells in order to screen/diagnose for a disease or disorder associated with changes in levels of VLA-1. The kit comprises an antibody that binds the antigen protein and means for indicating the reaction of the antibody with the antigen, if present. Various means for indicating the presence of an antibody can be used. For example, fluorophores, other molecular probes, or enzymes can be linked to the antibody and the presence of the antibody can be observed in a variety of routine ways. The method for screening for such disorders can involve the use of the kit, or simply the use of one of the disclosed antibodies and the determination of whether the antibody binds to VLA-1 in a sample. As will be appreciated by one of skill in the art, high or elevated levels of VLA-1 will result in larger amounts of the antibody binding to a target in the sample. Thus, degree of antibody binding can be used to determine how much VLA-1 is in a sample. Patients or samples with an amount of VLA-1 that is greater than a predetermined amount (e.g., an amount or range that a person without a VLA-1 mediated disease would have) can be characterized as having a VLA-1 mediated disorder.

In some embodiments, an article of manufacture is provided comprising a container, comprising a composition containing an anti-VLA-1 antibody, and a package insert or label indicating that the composition can be used to treat disease mediated by VLA-1. Preferably a mammal and, more preferably, a human, receives the anti-VLA-1 antibody. Preferably, such antibodies may be used for the treatment of disorders, such chronic inflammation and fibrosis disorders in which the VLA-1 activity level is elevated in one or more tissues as compared to a normal subject. Examples of such disorders are inflammatory disorders, such as immune-mediated inflammatory disorders (IMIDs), which are inflammatory conditions caused and sustained by an antigen-specific, pathological immune response. Among these disorders are various types of arthritis, such as rheumatoid arthritis, as well as allergic diseases, such as asthma, hay fever, and urticaria; different types of connective tissue disorders; inflammatory bowel disease (e.g., Crohn's disease and ulcerative colitis); insulin-dependent diabetes; uveitis; retinitis; graft rejection; and graft-versus host-disease. VLA-1 related disorders can also include tissue inflammation in infectious, ischemic, hemorrhagic, and traumatic conditions, e.g., fasciitis, stroke, infarction of the myocardium and other organs (e.g., lung and intestine), ARDS; hepatitis, (e.g., infectious and non-infectious, acute and chronic); acute and chronic pancreatitis; reperfusion injuries; radiation injuries; vascular restenosis of different types (e.g., coronary restenosis). Also included are osteoarthritis, osteoporosis, atherosclerosis, organ fibrosis, and neurodegenerative disorders such as Alzheimer's disease and multiple sclerosis. VLA-1 related disorders can include cancer, blood malignancies, e.g., leukemias and multiple myelomas; the development of a number of solid tumors, tumor growth, and metastatic spreading, or some combination thereof. In some embodiments, the VLA-1 related or mediated disorders is selected from the group of psoriasis, skin related conditions, allergic rhinitis, respiratory distress syndrome, bronchitis, tendinitis, bursitis, fever, migraine headaches, gastrointestinal conditions, periarteritis nodosa, thyroiditis, aplastic anemia, Hodgkin's Disease, rheumatic fever, autoimmune diseases, sarcoidosis, nephrotic syndrome, renal failure, Behcet's Syndrome, polymyositis, gingivitis, hypersensitivity, conjunctivitis, swelling occurring after injury, myocardial ischemia, endotoxin shock syndrome, renal scarring, and eosinophilic inflammation, or some combination thereof. The instructions can indicate the amount of a particular antibody to administer for any one of the above VLA-1 related disorders. As will be appreciated by one of skill in the art, this can depend upon the particular K_{D}, IC₅₀, and/or EC₅₀ of the antibodies, the particular disease to be treated, as well as the degree of treatment required. These particulars can readily be determined in each situation, by one of skill in the art, in light of the present disclosure.

Multiplexed Competitive Antibody Binning

In one embodiment, antibodies to a particular area of the VLA-1 protein are combined to be used together. The combination can be for different antibodies that are directed to the same location, or for a combination of different antibodies for different locations; thus, allowing multiple antibodies to bind simultaneously. One way that this can be examined is through multiplexed competitive antibody binning (MCAB).

The general method of MCAB is outlined in Jia et al., (J. Immunological Methods, 288:91-98 (2004), hereby incorporated in its entirety by reference). Basically, beads can be coupled with an antibody to capture a reference mAb. Each bead can have a unique spectral coding such that each mAb can be associated with a unique spectral address. All of the mAb bead complexes are pooled into a master mix and aliquotted into individual wells of microtiter plates. The master mix of reference antibody—bead complexes in each well can be incubated with antigen and then with a probe mAb, one different probe mAb per well. The probe antibody can be associated with a detectable marker. Any detectable marker can be used and the detectable marker can be associated with the probe antibody via any manner, for example through a detection antibody. The probe mAbs can bind to antigen that has been captured by a reference mAb if it recognizes a different epitope. The bound probe mAb can be detected in a number of ways. For example, through the use of biotin and streptavidin and read on a Luminex 100. Such detectable markers can also be used in other embodiments, such as a means for detecting the presence of an antibody of the amount of VLA-1 in a sample.

Epitope Mapping

In some embodiments, the particular epitope on VLA-1 to which the antibody binds is mapped. As will be appreciated by one of skill in the art, this can be done in a variety of ways and the particular approach is not critical. For example, competitive binding assays using antibodies that bind to a known location on the VLA-1 protein can be used. In other embodiments, mapping is achieved through altering various amino acids in the VLA-1 complex to determine how it alters binding of the antibody to VLA-1. By mapping those residues, which when altered result in a significant change in binding, to a structure of the VLA-1 complex, one is able to determine the most likely section of the VLA-1 protein involved in binding to the antibody. In some embodiments the alterations are achieved through point mutations on the VLA-1 protein. In other embodiments, chimeras of related VLA-1 proteins (e.g., human and mouse) are generated. In some embodiments, the paratope of the antibody is also determined in a similar fashion, by altering residues in the antibody and determining if the alterations have any impact on the binding of the antibody to the epitope.

Specificity of Antibodies

In some embodiments, the antibodies to VLA-1 are highly specific to VLA-1 over other VLA molecules, e.g., VLA-2. This higher specificity or selectivity can be any amount, for example, more than 1, 1-2, 2-5, 5-10, 10-100, 100-1000, 10³-10⁴, 10⁴-10⁶, 10⁶-10⁸, 10⁸-10¹⁰, 10¹⁰-10¹², 10¹²-10¹⁴ or greater fold more selective for VLA-1 than VLA-2. Selectivity can be determined via competition type assays, or simply by comparing the affinity of the antibody for VLA-1 to the affinity of the antibody for VLA-2. In other embodiments, selectivity can be for various variants of VLA-1. Thus, an antibody that binds to human VLA-1, but not mouse VLA-1 can also be created.

Assay for Blocking Binding to Collagen

The ability of these antibodies, or combinations of these antibodies to inhibit the interaction between VLA-1 and collagen can be assessed in a number of ways. In one embodiment, Chinese hamster ovary cells (“CHO cells”) are transfected with the human VLA-1 α chain (“CHO-VLA-1”). These cells and the anti-VLA-1 antibodies (i.e., VLA-1 antibodies) can be mixed together. Following this, the cells can be transferred to a collagen coated surface (e.g., BD BioCoat™ Collagen IV 96-well Microtest™ Plates (BD Biosciences, San Diego, Calif.), precoated with mouse collagen type IV Collagen). Binding of the cells to the collagen surface will indicate that the antibodies are not binding to VLA-1 in a manner to prevent VLA-1 binding to collagen. Either qualitative binding or the extent of any binding compared to a positive control can be used. The conditions of the test can be altered to simulate in vivo conditions.

Applying to Patient

Once an antibody has been identified that is fuinctional to block VLA-1 binding to collagen, it can then be administered to a patient in need of blocking VLA-1 binding. The amount to be administered will vary upon the individual, the K_{D}, or potency of the antibody, and the particular issue to be addressed. As the antibody is a human antibody directed to VLA-1, the risk of a HAMA response occurring is dramatically reduced and a relatively large amount and/or repeated administration can be permissible.

### EXAMPLES

The following examples, including the experiments conducted and results achieved are provided for illustrative purposes only and are not to be construed as limiting upon the teachings herein.

### Example 1

### Generation of Antibodies ti VLA-1

Fully human antibodies were generated to VLA-1 by immunizing various XenoMouse mice capable of producing antibodies of IgG2 or IgG4 isotype with various preparations containing all or part of the alpha 1 domain of VLA-1. This resulted in various hybridomas producing various antibodies to various parts of the alpha 1 domain of VLA-1.

Immunogens included “I domain-GST”, a recombinant GST fusion protein containing amino acids 127-340 of the human VLA-1 α1 subunit (shown in FIG. 1), expressed in E. coli. Alternatively or in combination with I domain-GST, CHO cells transfected with recombinant full-length human VLA-1 α1 subunit (“CHO-α1”) were used.

While Example 1 generally describes how the particular antibodies were created, the particulars of how antibodies can be created, in general (e.g., immunization and creation of the hybridomas) are known in the art and can be found, for example, in U.S. patent application Ser. No. 11/335907, filed Jan. 19, 2006, incorporated in its entirety by reference (especially relevant are examples 1 and 2). For example, initial immunization can be achieved by using an antigen to immunize XenoMouse® mice (U.S. Pat. No. 6,833,268, Issued Dec. 24, 2004 to Green et al., hereby incorporated by reference in its entirety), Abgenix, Inc. Fremont, Calif.). XenoMouse animals can be immunized via footpad route for all injections. The total volume of each injection can be 50 μl per mouse, 25 μl per footpad. VLA-1 antibody titers in the serum from immunized XenoMouse mice can be determined by ELISA.

Immunized mice can be sacrificed and the lymph nodes can be harvested. The lymphoid cells can be dissociated by grinding in DMEM to release the cells from the tissues, and the cells can be suspended in DMEM and washed. The fusion can be performed by mixing washed enriched B cells from above and nonsecretory myeloma (for example, P3X63Ag8.653 cells purchased from ATCC, cat. # CRL 1580 (Kearney et al., J. Immunol. 123, 1979, 1548-1550)) at a ratio of 1:1. The cell mixture can be gently pelleted by centrifugation at 800 g. After complete removal of the supernatant, the cells can be treated with 2-4 mL of Pronase solution (CalBiochem, cat. #53702; 0.5 mg/mL in PBS) for no more than 2 minutes. Then 3-5 ml of FBS can be added to stop the enzyme activity and the suspension can be adjusted to 40 mL total volume using electro cell fusion solution, ECFS (0.3 M Sucrose, Sigma, Cat # S7903, 0.1 mM Magnesium Acetate, Sigma, Cat # M2545, 0.1 mM Calcium Acetate, Sigma, Cat # C4705). The supernatant can be removed after centrifugation and the cells can be resuspended in 40 mL ECFS. This wash step can be repeated and the cells again can be resuspended in ECFS to a concentration of 2×10⁶ cells/mL.

Electro-cell fusion can be performed using a fusion generator, model ECM2001, Genetronic, Inc., San Diego, Calif. The following instrument settings can be used: alignment condition: voltage: 50 V, time: 50 s; membrane breaking at: voltage: 3000 V, time: 30 μsec; post-fusion holding time: 3 sec. The cells can be incubated for 15-30 minutes at 37° C., and then can be centrifuged at 400 g for five minutes. The cells can be gently resuspended in a small volume of Hybridoma Selection Medium (Hybridoma Culture Medium supplemented with 0.5×HA (Sigma, cat. # A9666)), and the volume can be adjusted appropriately with more Hybridoma Selection Medium, based on a final plating of 5×10⁶ B cells total per 96-well plate and 200 μL per well. The cells can be mixed gently and pipetted into 96-well plates and allowed to grow. On day 7 or 10, one-half the medium can be removed, and the cells can be re-fed with Hybridoma Selection Medium. Of course, the above is simply a general outline of how antibodies could be generated.

### Example 2

### Initial Analysis of Function of the Fully Human Antibodies

Antibodies were then examined for various functionalities. First, antibody binding to VLA-1 was examined via FACS, and antibody binding to CD49a I domain was examined by ELISA. The tests include a FACS analysis with various cells expressing various amounts or types of VLA, including IL-2 stimulated human or cynomolgus peripheral blood mononuclear cells (“human PBMC” and “cynomolgus PBMC”). A summary of some of these assays and results is shown in Table 2. “NT” indicates that it was Not Tested.

TABLE 2

| | FACS | | | | | |
|---|---|---|---|---|---|---|
| | | | | | CEM or | |
| | | | | | HT1080 human | |
| | | Human | Cynomolgus | Mouse | cell lines | ELISA |
| mAb | CHO-α1 | PBMC | PBMC | splenocytes | (VLA1−VLA2+) | I domain-GST |
| 1.7 | NT | ++ | + | + | − | + |
| 2.7 | NT | ++ | + | − | − | + |
| 2.19. | NT | ++ | + | − | − | + |
| 3.2 | NT | ++ | NT | NT | − | + |
| 3.9 | NT | ++ | + | − | − | + |
| 3.24 | NT | + | + | − | +/− | + |
| 3.40. | NT | ++ | + | − | +/− | + |
| 6.17. | Y | ++ | + | − | − | + |
| 6.18. | Y | ++ | + | − | − | + |
| 6.44. | Y | ++ | + | − | − | + |
| 6.48 | Y | ++ | + | − | − | + |
| 7.7 | Y | ++ | + | − | − | + |
| 7.11 | Y | NT | NT | − | NT | + |
| 7.34 | Y | ++ | + | − | − | − |
| 7.38 | Y | NT | NT | NT | NT | − |
| 7.57 | Y | + | + | − | NT | − |
| 7.73 | Y | + | + | − | NT | − |
| 10.1 | Y | + | + | − | − | − |

As can be seen in Table 2, several of the antibodies demonstrated binding to CHO-α1, Human PBMC and Cynomolgus PBMC. Few, if any, antibodies displayed binding ability towards mouse splenocytes. Additionally, several antibodies displayed no binding to VLA-2 expressing cell lines CEM or HT1080, e.g., 1.7, 2.7, 2.19, 3.2, and 3.9. This demonstrated that selective antibodies to VLA-1 can be generated that will not bind to other members of the VLA family. Additionally, many of the antibodies indicated an interaction with the I domain of VLA-1 via the ELISA test.

In addition to the binding assays, other functional assays were performed as well, such as various potency tests.

An “adhesion assay,” using a fixed concentration of mAb was performed with the various antibodies. The data represent an average of three assays and are presented as neutralizing ability relative to the control neutralizing α1 mAb, FB2 (=, similar; >, better than control; <, worse than control; 0, little or no inhibition). Thus, a “>” indicates that the antibody is more effective than the control in neutralizing the ability of CD49a to bind to T cells. The results are summarized in Table 3 below.

In other assays, the ability of XenoMouse antibodies to inhibit binding of I domain-GST was tested, the results of which are shown in Table 3. These were competitive binding assays in which the mAbs were tested for their ability to inhibit the interaction between immobilized collagen IV and I domain-GST. This was achieved by determining the amount of I domain-GST bound to collagen IV with or without a 1 hour preincubation with the XenoMouse antibodies. Detection was achieved through the use of either streptavidin-HRP (in the case of biotinylated I domain-GST, i.e., “I domain-btn”) or anti-GST-HRP (when using non-biotinylated I domain-GST). Because Mn²⁺ or Mn²⁺ cations stabilize the ‘active’ conformation of the VLA-1 I domain, all incubations after the initial addition of the I domain were in buffer containing 1 mM MnCl₂. Results were compared to those obtained with a control anti-al antibody 5E8D9. The results are summarized in Table 3.

The potency of the antibodies was examined in a T cell adhesion assay, the results of which are also presented in the last column of Table 3. In this assay, IL-2 stimulated T cells from human blood were preincubated with titrated amounts of selected XenoMouse antibodies and then tested for their ability to bind to immobilized collagen IV. Results were compared to those obtained with a control anti-α1 antibody FB2. Because of day-to-day and donor-to-donor variability in the assay, the results are summarized here as relative estimated IC₅₀ averaged from 4 assays, with “+” signifying >5 nM estimated IC₅₀, and “+++” representing the best inhibition. The results of this test are displayed in Table 3.

TABLE 3

| | |
|---|---|
| Inhibition | Potency |

| | T cells | I domain-btn | I domain | |
|---|---|---|---|---|
| mAb | (33 nM Ab) | IC₅₀ (nM) | IC₅₀ (nM) | T cells |
| 1.7. | = | 2.6 | 3.7 | +++ |
| 2.7. | > | 2.4 | 6.4 | +++ |
| 2.19. | = | 3.0 | 7.4 | ++ |
| 3.2. | < | 1.6 | 3.2 | NT |
| 3.9. | =/< | 4.6 | 12 | + |
| 3.24 | 0 | NT | NT | NT |
| 3.40. | = | 23 | 14 | + |
| 6.17. | = | 7.6 | 12 | +++ |
| 6.18. | = | 3.3 | 7.6 | ++ |
| 6.44. | = | 2.7 | 7.2 | +++ |
| 6.48 | = | 6.1 | 9.8 | ++ |
| 7.7. | =/< | 7 | 9.9 | NT |
| 7.11. | =/< | 2.4 | 4.2 | + |
| 7.34. | = | 4.5 | 7.5 | ++ |
| 7.38. | 0/< | 5.5 | 23 | NT |
| 7.57. | 0/< | NT | NT | NT |
| 7.73 | 0/< | 5.0 | 8.2 | NT |
| 10.1 | = | 3.1 | 5.5 | + |

### Example 3

### VLA-1 Mediated Adhesion Assay

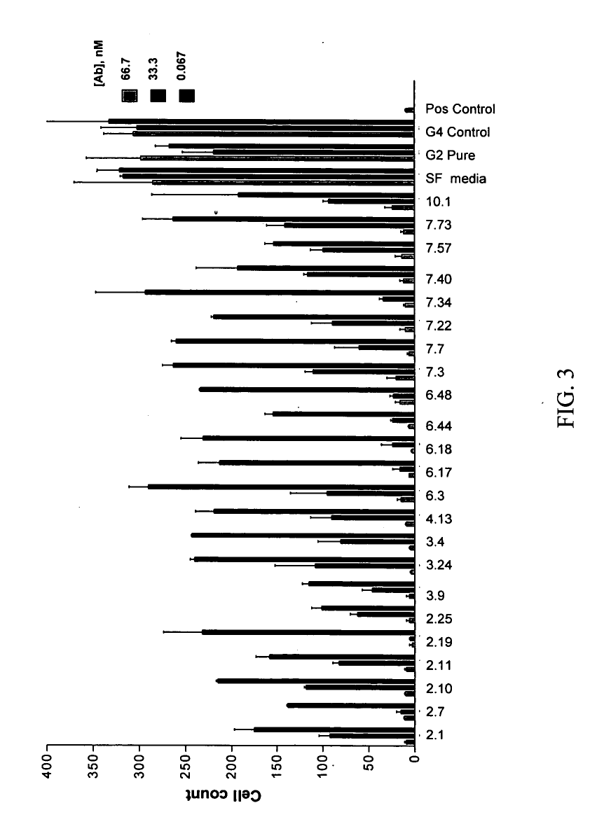

A VLA-1 mediated adhesion assay was performed. This example demonstrates one method of performing a VLA-1-Collagen IV adhesion assay. Chinese hamster ovary cells (“CHO-KI”) and CHO-KI cells transfected with the human VLA-1 α chain (“CHO-VLA-1”) were detached using tissue culture grade trypsin at 37° C. After 10 minutes, an equal volume of tissue culture media containing 10% fetal calf serum (FCS; CellGro) was added. This detachment protocol was previously determined not to significantly affect binding of anti-VLA-1 control antibodies to the transfectant CHO-VLA-1 cells. Cells were suspended in DMEM F12 containing 0.7 mM MgSO₄, pH 6.5±0.5, with or without 10% FCS. An equal volume of cells and purified anti-VLA-1 antibodies were mixed in 96-well V-bottom plates. A murine IgG2a anti-human VLA-1 antibody, 5E8D9 (Upstate Biotechnologies, Lake Placid, N.Y.), served as a positive control and is designated “PosCtrl” in FIG. 3.

Human IgG isotype-matched irrelevant antibodies were used as negative controls and did not exhibit any blocking activity in any of the assays. The cells were incubated with the antibodies at 37° C. for approximately five hours, after which they were transferred to BD BioCoat™ Collagen IV 96-well Microtest™ Plates (BD Biosciences, San Diego, Calif.), precoated with mouse collagen type IV Collagen. Antibodies were tested in duplicate. The cells were allowed to adhere at room temperature for 30 minutes. At this time, nonadherent cells were removed with several washes with serum-free medium. Adherent cells were detached using trypsin, as described above, and CentriRed nuclear dye was added to the cell suspensions. The cells were transferred to 384-well plates and cell numbers determined using Fluoremetric Microvolume Assay Technology (FMAT).

Purified antibodies were tested in three separate assays to determine their relative potencies. Antibodies were tested in the following concentration ranges: Assay 1: 3 concentrations ranging from 67 nM to 62 pM (results are displayed in FIG. 3); Assay 2: 6 concentrations ranging from 0.62 pM to 62 nM; and Assay 3: 6 concentrations ranging from 75 pM to 187 nM.

The percent inhibition was calculated for each experiment and antibody concentration. Zero inhibition was defined by the isotype control. The resulting 8-dilution dose curve, consolidated from all 3 experiments, was plotted on GraphPad Prism. Antibodies were tested from 62 pM to 187 nM. The IC₅₀ values were determined for each antibody by the Prism Graphpad software. The results are shown in Table 4 below. Hill slope and R² values are shown with their respective IC₅₀s. Values with acceptable Hill slope values ranged from 0.5 to 1.5) and values with acceptable R² values were above 0.8. Samples are grouped by decreasing potency in the adhesion assay.

TABLE 4

| | | | | 95% Confidence Intervals |
|---|---|---|---|---|
| Clone# | HILL SLOPE | R² | IC50 (nM) | IC₅₀, M |
| 4.13 | 0.20 | 0.96 | <0.01 | — |
| 2.11 | 0.35 | 0.86 | 0.07 | — |
| 2.7 | 0.84 | 0.91 | 0.08 | 2.3e−012 to 2.6e−009 |
| 6.44 | 0.83 | 0.95 | 0.08 | 7.9e−012 to 8.8e−010 |
| 6.48 | 1.43 | 0.94 | 0.12 | 3.3e−011 to 4.7e−010 |
| 7.34 | 0.78 | 0.99 | 0.19 | 8.5e−011 to 4.1e−010 |
| 6.18 | 0.91 | 0.99 | 0.19 | 8.1e−011 to 4.6e−010 |
| 6.17 | 0.81 | 0.97 | 0.27 | 6.2e−011 to 1.2e−009 |
| 3.9 | 1.55 | 0.99 | 0.38 | 1.8e−010 to 7.9e−010 |
| 7.22 | 0.69 | 0.95 | 0.41 | 3.7e−011 to 4.4e−009 |
| 7.73 | 0.54 | 0.99 | 0.43 | 1.1e−010 to 1.7e−009 |
| 2.19 | 1.75 | 1.00 | 0.47 | 3.2e−010 to 6.9e−010 |
| 3.24 | 0.76 | 0.99 | 0.58 | 2.8e−010 to 1.2e−009 |
| 7.7 | 1.31 | 1.00 | 0.59 | 4.1e−010 to 8.6e−010 |
| 6.3 | 0.59 | 0.81 | 0.68 | 3.1e−012 to 1.5e−007 |
| 7.3 | 0.74 | 0.97 | 0.94 | 2.3e−010 to 3.9e−009 |
| 10.1 | 0.84 | 0.98 | 1.00 | 3.9e−010 to 2.6e−009 |
| 3.40 | 2.11 | 0.97 | 1.30 | 5.4e−010 to 3.1e−009 |
| 2.10 | 0.58 | 0.89 | 2.37 | 1.8e−011 to 3.1e−007 |
| 7.57 | 1.34 | 0.91 | 3.25 | 7.8e−010 to 1.4e−008 |
| 7.40 | 0.10 | 0.70 | 4.72 | — |
| 2.1 | 0.40 | 0.65 | 21.35 | — |
| 2.25 | 0.25 | 0.68 | 74.86 | — |

All antibodies tested inhibited VLA-1 mediated adhesion to Collagen IV with IC₅₀ values ranging from low pM to low nM. The most potent antibodies had an IC₅₀ value that was less than 500 pM and a relative inhibition, determined at an optimal concentration within each of the experiments, generally greater than 80% (4.13, 2.11, 2.7, 6.44, 6.48, 7.34, 6.18, 6.17, 3.9, 7.22, 7.73, 2.19).

### Example 4

### Affinity Determination

The kₐ, k_{d}, and K_{D} were determined for the above antibodies. Anti-human IgG was immobilized on all four surfaces of CM5 sensor chip using standard amine coupling chemistry. Based on the supplied antibody concentrations all mAb samples were diluted to a concentration of 5 μg/ml and injected for 4 minutes to capture onto the sensor surface. GST-linked VLA-1 I domain was diluted 1:5000 given a concentration of 19.45 nM, and injected over the mAb surfaces for 6 minutes association phase, followed by a 20 minute dissociation phase. Bound complexes were regenerated with a 20 s injection of 1:100 dilution of phosphoric acid. The running buffer consisted of 10 mM HEPES, 150 mM NaCl, 0.005% p20 and 0.1 mg/ml BSA, pH 7.4 at 23 degrees C. In a duplicate screen, the buffer contained 1 mM MgCl₂. The antigen response data were fit to a 1:1 interaction model. Rate constants are listed in Table 5. In a subsequent screen of selected monoclonal antibodies, each mAb was captured onto an anti-IgG surface and GST-linked VLA-1 I domain was diluted 1:15000 to a starting concentration of 6.5 nM, followed by a three-fold dilution series. The rate constants from this screen are noted in Table 5 with an asterisk next to the K_{D} value.

TABLE 5

| | |
|---|---|
| Buffer contains no Mg²⁺ | Buffer contains 1 mM Mg²⁺ |

| mAb | kₐ (M⁻¹s⁻¹) | k_{d} (s⁻¹) | K_{D} (pM) | kₐ (M⁻¹s⁻¹) | k_{d} (s⁻¹) | K_{D} (pM) |
|---|---|---|---|---|---|---|
| 1.7 | | | | 5.15E+05 | 7.62E−05 | 148* |
| 2.1 | 4.56E+05 | 2.11E−03 | 4625 | | | |
| 2.7 | 1.34E+05 | 8.22E−05 | 61* | 3.21E+05 | 3.77E−05 | 117* |
| 2.10 | 1.44E+05 | 1.47E−04 | 1021 | | | |
| 2.11 | 2.34E+05 | 2.08E−04 | 889 | 3.18E+05 | 8.00E−06 | 25* |
| 2.19 | 1.24E+05 | 5.93E−05 | 478 | 5.38E+05 | 1.31E−04 | 243 |
| 2.25 | 7.58E+04 | 1.11E−04 | 1464 | 8.30E+03 | 1.30E−05 | 1566 |
| 3.2 | | | | 3.54E+05 | 1.43E−05 | 40 |
| 3.9 | 1.15E+05 | 3.42E−05 | 296* | 3.32E+05 | 1.31E−05 | 39* |
| 3.24 | 2.29E+04 | 3.75E−04 | 16376* | 2.14E+05 | 9.96E−05 | 465 |
| 3.40 | 5.60E+03 | 1.30E−04 | 23214* | 2.51E+05 | 1.20E−05 | 48 |
| 4.13 | 9.80E+04 | 1.44E−04 | 1500* | | | |
| 5.3 | | | | 3.51E+05 | 4.87E−04 | 1387 |
| 6.3 | 1.44E+05 | 2.43E−04 | 1688 | | | |
| 6.17 | 9.46E+04 | 1.54E−04 | 1627 | 5.33E+05 | 1.36E−04 | 255* |
| 6.18 | 1.23E+05 | 7.08E−05 | 574* | 5.23E+05 | 7.20E−05 | 138* |
| 6.44 | 2.47E+05 | 8.64E−05 | 349* | 5.53E+05 | 5.94E−05 | 107* |
| 6.46 | | | | 2.53E+04 | 3.38E−04 | 13381 |
| 6.48 | | | | 5.61E+05 | 1.21E−04 | 216* |
| 6.54 | | | | 4.97E+03 | 3.98E−04 | 80129 |
| 7.3 | 9.88E+04 | 1.50E−04 | 1519 | | | |
| 7.4 | | | | 4.97E+03 | 3.98E−04 | 80129 |
| 7.7 | 1.57E+05 | 4.69E−05 | 298* | 3.16E+05 | 3.67E−05 | 116 |
| 7.22 | 2.53E+05 | 1.37E−04 | 541 | | | |
| 7.34 | 2.03E+04 | 1.12E−04 | 5523* | 1.08E+05 | 4.20E−04 | 3889 |
| 7.40 | | | | 2.53E+04 | 3.38E−04 | 13381 |
| 7.46 | | | | 3.90E+04 | 9.95E−05 | 2551 |
| 7.57 | | | | 3.64E+05 | 3.36E−05 | 92* |
| 7.73 | 4.95E+04 | 1.48E−04 | 2992 | 4.11E+04 | 1.79E−04 | 4355 |
| 10.1 | 1.66E+05 | 1.73E−04 | 1042 | 4.53E+05 | 3.10E−05 | 68* |

As can be seen from the data in Table 5, the antibodies displayed K_{D} from 1566 pM to 25 pM, in part depending upon which protocol was used. As will be appreciated by one of skill in the art, the amount of Mg2+ can be varied and can include, for example, an amount between 10 nM and 100 mM and/or 100 nM and 10 mM.

### Example 5

### Multiplexed Competitive Antibody Binning

The following method describes how to characterize mAb through Multiplexed Competitive Antibody Binning (MCAB). The general method is outlined in Jia et al., (J. Immunological Methods 288:91-98 (2004), hereby incorporated in its entirety by reference). Multiplexing Luminex beads were coupled with a mouse anti-human IgG mAb to capture a reference mAb. Each bead had a unique spectral coding such that each mAb was associated with a unique spectral address. All of the mAb bead complexes were pooled into a master mix and aliquotted into individual wells of 96-well microtiter plates. The master mix of reference antibody-bead complexes in each well was incubated first with antigen, then with a probe mAb, one different probe mnAb per well. The antigen in the competitive antibody binning assay was the recombinant I domain. The probe antibody is also associated with a detectable marker, in this example, via a detection antibody that is attached to the detectable marker (e.g., a fluorescent label). The probe mAbs only bound to antigen that had been captured by a reference mAb that recognized a different epitope. The bound probe mAb was detected by the interaction of streptavidin-PE and the biotinylated version of the same mouse mAb used to capture the reference mAb. The signal was read as RFU on a Luminex 100. See Table 6 for results.

TABLE 6

| Bin#1 | Bin#2 | Bin#3 | Bin#4 | Bin#5 | Bin#6 | No Binding |
|---|---|---|---|---|---|---|
| 2.25 | 2.1 | 2.7 | 2.14 | 2.5 | 2.12 | 3.13 |
| 3.16 | 2.3 | 2.18 | 3.5 | 2.10 | 3.10 | 3.41 |
| 3.17 | 2.17 | 1.4 | 3.7 | 3.24 | | 4.1 |
| 3.18 | 2.19 | 1.5 | 3.9 | 3.38 | | 5.4 |
| 3.19 | 2.21 | 1.7 | 3.21 | | | 6.6 |
| 3.4 | 2.23 | 1.8 | 3.40 | | | 6.43 |
| 4.2 | 2.26 | 2.8 | 4.3 | | | 7.27 |
| 2.4 | 2.27 | 2.11 | 4.8 | | | |
| 2.6 | 3.1 | 2.13 | 6.30 | | | |
| 2.9 | 3.25 | 2.24 | 6.31 | | | |
| 2.20 | 3.30 | 3.2 | 6.58 | | | |
| 2.22 | 3.36 | 3.6 | 7.3 | | | |
| 2.28 | 4.5 | 3.8 | 7.7 | | | |
| 2.29 | 4.15 | 3.11 | 7.9 | | | |
| 3.3 | 6.2 | 3.12 | 7.32 | | | |
| 3.14 | 6.10 | 3.20 | 7.48 | | | |
| 3.15 | 6.44 | 3.23 | 7.52 | | | |
| 3.22 | 7.4 | 3.26 | 7.57 | | | |
| 3.28 | 7.11 | 3.27 | 7.60 | | | |
| 3.31 | 7.14 | 3.29 | 7.64 | | | |
| 3.33 | 7.22 | 3.32 | 7.65 | | | |
| 3.34 | 7.24 | 3.39 | 7.69 | | | |
| 3.35 | 7.38 | 4.4 | 7.72 | | | |
| 3.37 | 7.40 | 5.2 | | | | |
| 4.9 | 7.46 | 5.3 | | | | |
| 4.10 | 7.66 | 6.26 | | | | |
| 4.11 | 7.73 | 7.16 | | | | |
| 4.12 | | 7.19 | | | | |
| 4.13 | | 7.25 | | | | |
| 4.14 | | 7.34 | | | | |
| 4.16 | | 7.37 | | | | |
| 5.1 | | 7.71 | | | | |
| 6.1 | | 7.50 | | | | |
| 6.3 | | | | | | |
| 6.7 | | | | | | |
| 6.8 | | | | | | |
| 6.9 | | | | | | |
| 6.12 | | | | | | |
| 6.13 | | | | | | |
| 6.15 | | | | | | |
| 6.17 | | | | | | |
| 6.18 | | | | | | |
| 6.19 | | | | | | |
| 6.21 | | | | | | |
| 6.22 | | | | | | |
| 6.23 | | | | | | |
| 6.28 | | | | | | |
| 6.32 | | | | | | |
| 6.33 | | | | | | |
| 6.37 | | | | | | |
| 6.42 | | | | | | |
| 6.46 | | | | | | |
| 6.47 | | | | | | |
| 6.53 | | | | | | |
| 6.54 | | | | | | |
| 6.55 | | | | | | |
| 6.57 | | | | | | |
| 7.1 | | | | | | |
| 7.2 | | | | | | |
| 7.6 | | | | | | |
| 7.8 | | | | | | |
| 7.10 | | | | | | |
| 7.13 | | | | | | |
| 7.15 | | | | | | |
| 7.17 | | | | | | |
| 7.18 | | | | | | |
| 7.20 | | | | | | |
| 7.21 | | | | | | |
| 7.23 | | | | | | |
| 7.26 | | | | | | |
| 7.35 | | | | | | |
| 7.36 | | | | | | |
| 7.49 | | | | | | |
| 7.56 | | | | | | |
| 7.63 | | | | | | |
| 7.67 | | | | | | |
| 7.70 | | | | | | |

As can be seen in Table 6 above, the antibodies separated into six different bins and a no binding grouping.

As will be appreciated by one of skill in the art, in some embodiments, antibodies that belong to a particular bin are particularly of interest. Put another way, antibodies that cross-compete in their binding to the antigen can also be of interest. This information can allow the characterization and grouping of antibodies as similar, or allowing the mixing of antibodies together so that multiple antibodies can bind to the antigen without competition from one another. In one aspect, antibodies that cross-compete with antibodies in bin 3 are the invention. Examples of such antibodies would be any antibody in bin 3, in Table 6, with any other antibody in bin 3. Additionally, other antibodies that also cross-compete for the same binding area on the antigen would also be included. In other embodiments, the antibodies of interest cross-compete with the antibodies selected from the group consisting of bin 1, bin 2, bin 3, bin 4, bin 5, and bin 6. All that is meant by cross-compete is that both antibodies can specifically bind to the antigen and that either one can block the binding of the other.

### Example 6

### Sequencing and Characterization

The antibodies were sequenced, the framework regions of VH and VL determined and epitope mapping then performed. Both the amino acid and nucleic acid sequences for the various light and heavy chains are presented in Table 1 above. Table 7 below summarizes the various sections of the antibodies, as well as FIGS. 4A-4H and FIGS. 4I-4P.

TABLE 7

| | Sequences | | | | | |
|---|---|---|---|---|---|---|
| | | | | | | FRM |
| mAb | VH | DH | JH | VL | JL | (HC/LC) |
| 1.7 | 4-31 | — | 6B | A27 | JK4 | 0/1 |
| 2.7 | 3-15 | — | 6B | O12 | JK1 | 1/0 |
| 3.9 | 3-33 | 3-10 | 6B | V2-1 | JL2 | 0/3 |
| 6.17 | 5-51 | 2-15 | 4B | A27 | JK1 | 3/2 |
| 6.18 | 5-51 | 2-15 | 4B | A27 | JK1 | 1/2 |
| 6.44 | 4-31 | 3-3 | 4B | B3 | JK1 | 1/2 |
| 6.48 | 5-51 | 2-15 | 4B | A1 | JK4 | 3/3 |
| 7.7 | 1-2 | 3-9 | 5B | B3 | JK2 | 0/2 |
| 7.34 | 3-15 | — | 6B | O12 | JK3 | 0/2 |
| 10.1 | 4-31 | 6-19 | 4B | A27 | JK4 | 3/1 |

The last column in Table 7 displays the number of mutations in the framework heavy chain (HC) and light chain (LC) regions.

### Example 7

### Epitope Mapping

A relevant epitope on CD49a was determined. Antibodies from twelve distinct hybridoma lineages were analyzed and included antibodies 1.7, 2.7, 2.11, 2.19, 3.9, 3.40, 5.3, 6.17, 6.18, 6.44, 6.48, and 7.7.

Initially, it was determined that all antibodies bound recombinant human CD49a I domain and recognized conformational, possibly sequence-discontinuous, epitopes.

Crossreactivity of the XenoMouse antibodies to mouse and rat CD49a was assessed using recombinantly expressed proteins representing the CD49a I domain sequence from these organisms. Amplification of mouse and rat CD49a I domain RNA sequence was from mouse thymus and rat liver, respectively. The CD49a I domains were cloned into pSep4 vector as a V5, His-fusion protein using the immunoglobulin kappa (Igκ) signal peptide sequence. Human VLA-1 I domain was amplified and cloned into a pSecTAg vector as a myc-, His-fusion protein, using the Igic signal peptide sequence. All recombinant I domains were expressed in 293T cells, and XenoMouse antibody binding to the secreted I domains was assessed using an enzyme-linked immunosorbent assay (ELISA). Variants of I domains, in which selected residues in the mouse sequence were substituted for the corresponding human residues, were also expressed, and tested for XenoMouse antibody binding by ELISA. An alignment of human, mouse, and rat I domain sequences is shown in FIG. 2.

Because of the striking cluster of nonhomologous residues in the area boxed in FIG. 2, the effect of substituting residues in region 91-96 of the mouse I domain (GRRGGL, SEQ ID NO: 418) for the corresponding human sequence VQRGGR (SEQ ID NO: 419) was tested. This substitution did not confer binding by any of the XenoMouse antibodies tested. Conversely, when residues 91-96 of the human CD49a I domain (VQRGGR) were replaced with the corresponding mouse sequence, GRRGGL, it reduced the binding of antibodies 2.7, 2.19, 6.17, 6.18, 6.44, and 6.48. Thus, this region of the I domain is necessary, but not sufficient for I domain recognition by these antibodies.

Additional mutagenesis of the human I domain was carried out in order to further identify the critical residues for antibody binding. Four nonhomologous mouse I domain residues in the amino terminus (I1 IF, E48K, E66A and K88N) and three in the carboxy terminus (KI42Q, S162H and T204A) were substituted into the human I domain sequence, and binding of the selected XenoMouse antibodies to each of the mutants was determined by ELISA.

The results indicated that positions 11 and 88 are not directly involved in binding of any of the twelve XenoMouse antibodies to the CD49a I domain. In addition, the T204A mutation did not affect the binding of 11 out of 12 antibodies (clone 6.48 was not analyzed). However, the E48K mutation abolished the binding of antibodies 3.9, 3.40, and 7.7. Binding of antibodies 6.17 and 6.18 was affected by the S162H mutation, and antibodies 1.7, 2.11, and 5.3 were all sensitive to the I domain mutation K142Q. The results are summarized in Table 8.

TABLE 8

| | Mutation | | | | | | | |
|---|---|---|---|---|---|---|---|---|
| mAb | I11F | E48K | E66A | K88N | V91G, Q92R, R96L | K142Q | S162H | T204A |
| 3.9 | + | NB | + | + | + | + | + | + |
| 3.40 | + | NB | + | + | + | + | + | + |
| 7.7 | + | NB | + | + | + | + | + | + |
| 6.44 | + | + | + | + | NB | + | + | + |
| 2.7 | + | + | NB | + | NB | + | + | + |
| 2.19 | + | + | NB | + | NB | + | + | + |
| 6.17 | + | + | + | + | NB | + | NB | + |
| 6.18 | + | + | + | + | NB | + | NB | + |
| 6.48 | + | + | + | + | NB | + | + | n.d. |
| 1.7 | + | + | + | + | + | NB | +/− | + |
| 2.11 | + | + | + | + | + | NB | +/− | + |
| 5.3 | + | + | + | + | + | NB | +/− | + |

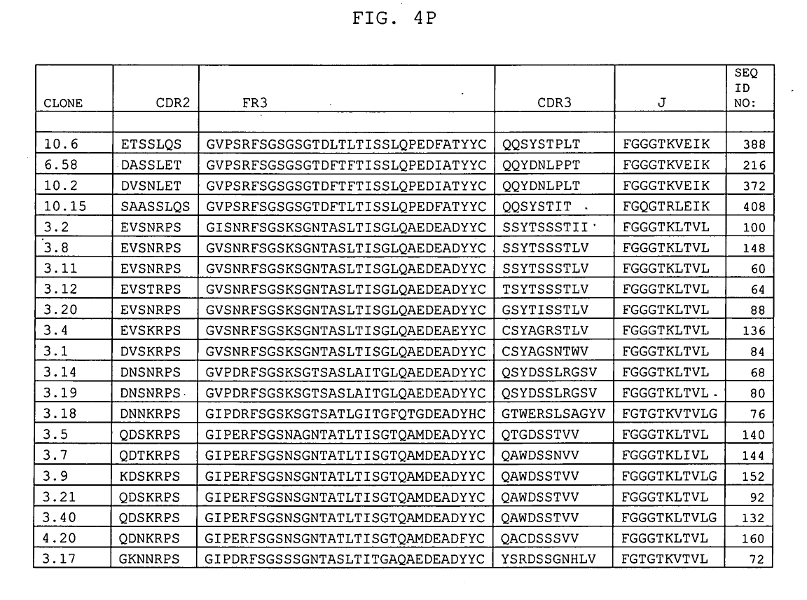

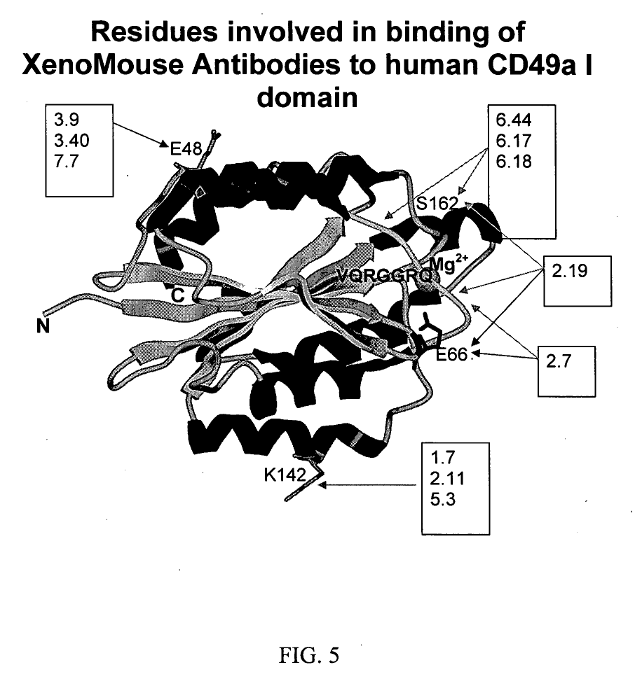

The residues critical for XenoMouse antibody recognition are highlighted in the structural model of the human VLA-1 I domain, shown in FIG. 5.

### Example 8

### Assay of VLA-1 MABS in PMA-Stimulated Adhesion of IL-2 Activated T Cells to Collagen

The ability of the antibodies to prevent adhesion mediated by VLA-1 was then tested by another approach. The method involved T cells that had been pre-activated by culture in interleukin-2 and transiently activated with phorbol myristate acetate, a known enhancer of integrin function (see Roberts et al., Immunology 97:679-685 (1999), herein incorporated by reference in its entirety). This allows one to study the ability of the antibodies to block adhesion to collagen in the presence of a T cell stimulus. As will be appreciated by one of skill in the art, such a stimulus, under physiological conditions, could be mediated by an interaction with another cell, binding of a cytokine, etc. Thus, this method allows one to simulate other in vivo like conditions and to determine the strength of the antibodies under those conditions.

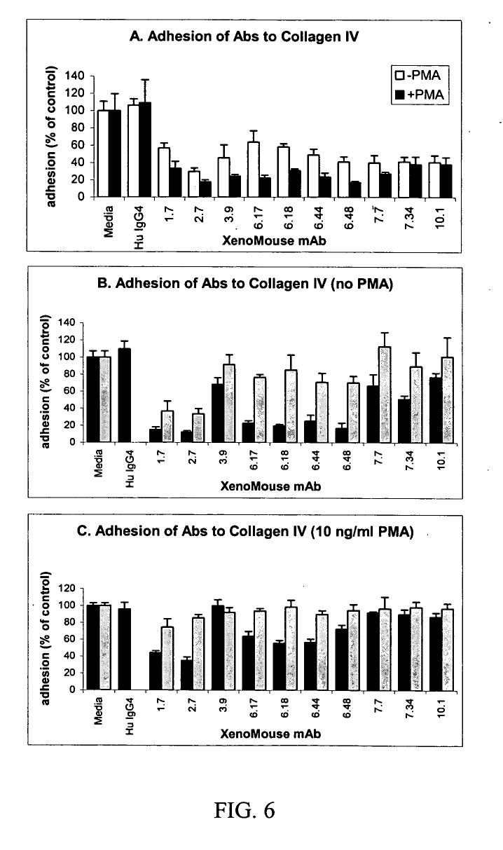

Human Collagen IV (Sigma, St Louis, Mo.) was dissolved in 0.1M acetic acid at 1 mg/ml for 2-3 hours at 4° C. Thereafter, it was diluted to 50 μg/ml and used to coat 96 well flat bottom plates overnight. The following day, excess collagen was removed and the plates were blocked with 3% BSA for lhr at room temperature; Anti-VLA-1 and control antibodies were diluted in serum free RPMI media containing 1% BSA. Human peripheral blood mononuclear cells that had been activated in vitro for several days with interleukin-2 were plated at 300,000 cells per well in V-bottom plates, pelleted, and resuspended in 100 ul of ice-cold diluted antibody or BSA-containing media alone. Each condition was prepared in duplicate, and one set of conditions contained 10 ng/ml phorbol myristate acetate (PMA). After washing the blocked, collagen coated, plates twice with PBS, the cells were transferred to these plates and allowed to settle, on ice, for 20 minutes. The plates were then transferred to a 37° C. and 5% CO₂ environment for 30 minutes. Nonadherent cells were washed away, and plates were frozen at −80° C. for 30-60 minutes. The plates were then thawed and CyQuant GR dye in cell lysis buffer was added to each well. The plates were read on a fluorometer at 485 nm excitation and 530 nm emission with 10 flashes, 40 microsecond integration time, optimal gain and 0 lag time. The results are displayed in FIGS. 6A-6C. In FIG. 6A, antibodies (10 micrograms/ml final concentration) were tested for their ability to inhibit adhesion of IL-2 activated human peripheral blood T cells to Collagen IV. The white bars represent no PMA and the black bars represent 10 ng/ml PMA. As can be seen in the figure, most of the present antibodies demonstrated inhibition characteristics even when PMA stimulation converted presumably most of the VLA-1 molecules on the T cells to an active state, suggesting that these mAbs are able to inhibit T cell adhesion in vivo in the face of potent VLA-1 stimuli such as T cell receptor ligation or chemokine receptor ligation.

In FIG. 6B, antibodies were tested at 5 nM (black bars) or 500 pM (grey bars) concentrations in an adhesion assay, in the absence of 10 ng/ml PMA. In FIG. 6C, the antibodies were tested at 5 nM (black bars) or 500 pM (grey bars) concentrations in an adhesion assay in the presence of 10 ng/ml PMA. Results are presented as the percent of adhesion in the absence of antibody, and are averages ± SEM for at least four replicates. The actual percents for the results displayed in FIGS. 6A-6C are also displayed in Tables 9-11 respectively.

TABLE 9

| | −PMA | Std dev | +PMA | Std dev |
|---|---|---|---|---|
| Media | 100.00 | 10.92 | 100.00 | 19.64 |
| Hu | 106.49 | 7.31 | 109.26 | 26.75 |
| IgG4 | | | | |
| 1.7 | 57.05 | 5.72 | 33.77 | 7.87 |
| 2.7 | 29.97 | 4.16 | 18.04 | 2.35 |
| 3.9 | 45.82 | 14.95 | 24.61 | 2.01 |
| 6.17 | 64.08 | 13.01 | 22.54 | 3.21 |
| 6.18 | 58.46 | 3.85 | 31.25 | 1.97 |
| 6.44 | 49.24 | 6.73 | 23.71 | 4.39 |
| 6.48 | 41.27 | 5.79 | 17.48 | 1.51 |
| 7.7 | 40.08 | 8.57 | 27.38 | 2.07 |
| 7.34 | 41.34 | 5.48 | 38.33 | 8.80 |
| 10.1 | 40.90 | 7.70 | 38.40 | 8.23 |

TABLE 10

no PMA

| | 5 nM | | 0.5 nM | Std |
|---|---|---|---|---|
| | mAb | Std dev | mAb | dev |
| Media | 100.00 | 7.05 | 100.00 | 7.05 |
| Hu IgG4 | 09.50 | 8.99 | | |
| 1.7 | 14.77 | 3.42 | 36.88 | 11.59 |
| 2.7 | 12.05 | 1.67 | 33.46 | 6.05 |
| 3.9 | 68.00 | 8.07 | 91.45 | 11.29 |
| 6.17 | 22.33 | 3.23 | 76.26 | 3.42 |
| 6.18 | 19.70 | 1.51 | 85.03 | 17.64 |
| 6.44 | 24.97 | 7.14 | 70.69 | 10.61 |
| 6.48 | 16.70 | 6.13 | 70.03 | 7.78 |
| 7.7 | 66.49 | 13.46 | 112.62 | 16.51 |
| 7.34 | 50.56 | 4.29 | 89.20 | 16.49 |
| 10.1 | 76.56 | 4.91 | 100.63 | 22.69 |

TABLE 11

with 10 ng/ml PMA

| | 5 nM | Std | 0.5 nM | Std |
|---|---|---|---|---|
| | mAb | dev | mAb | dev |
| Media | 100.00 | 2.99 | 100.00 | 2.99 |
| Hu | 95.60 | 7.96 | | |
| IgG4 | | | | |
| 1.7 | 44.14 | 2.48 | 74.49 | 9.79 |
| 2.7 | 34.82 | 4.43 | 85.37 | 4.04 |
| 3.9 | 99.62 | 7.16 | 91.94 | 5.73 |
| 6.17 | 63.73 | 5.74 | 93.73 | 2.87 |
| 6.18 | 55.53 | 3.34 | 98.43 | 7.97 |
| 6.44 | 56.64 | 3.78 | 89.79 | 4.34 |
| 6.48 | 72.47 | 4.78 | 94.58 | 6.96 |
| 7.7 | 91.91 | 1.12 | 96.59 | 13.81 |
| 7.34 | 89.66 | 5.74 | 98.18 | 6.52 |
| 10.1 | 86.81 | 4.77 | 96.72 | 6.04 |

### Example 9

### Treatment of a Disorder by Administering an Antibody

A patient suffering from a VLA-1 mediated disorder involving over-expression of VLA-1 is identified. A dosage of 5 mg/kg of a VLA-1 antibody described herein that inhibits the binding of VLA-1 to collagen is administered by intravenous injection to the patient. A booster administration is given three weeks later, and every three weeks thereafter. The VLA-1 antibody causes a partial or complete inhibition of adhesion of VLA-1 to collagen, thereby reducing the VLA-1 mediated disorders.

### Example 10

### Method of Identifying and Antibody that Can Prevent or Reduce the Symptoms of Asthma

The allergenic sheep model described in Abraham et al., (Am J. Respir. Crit. Care Med., 169:97-104 (2004)), hereby incorporated in its entirety by reference, can be used as test subjects, and means for testing antibodies to determine which are functional in preventing the symptoms of asthma. Thus, antibodies which reduce the symptoms of disease in the sheep in comparison to control healthy sheep will be those that can prevent or reduce the symptoms of asthma in individuals.

In alternative embodiments, rodent models, such as that described in De Fougerolles et al. (J. Clin. Invest., 105:721-729 (2000)) hereby incorporated in its entirety by reference, can be used to test the ability of antibodies to inhibit either the above diseases themselves or the symptoms of the diseases.

Additionally, nonhuman primate models, as taught by Mikara et al., (Clin. Immunol., 98:319-326 (2001)), hereby incorporated in its entirety by reference, can be used to determine if the particular antibody is effective for its particular purpose. Similarly, a human xenograft model, as taught by Conrad et al., (34^{th} Annual European Society for Dermatological Research Meeting, Abstract No: 152, 9-11 September 2004, Vienna Austria), hereby incorporated in its entirety by reference, can be used to determine if a particular antibody will be useful in preventing psoriasis. In each of these examples, a set amount of the antibody is administered to the model system. The reduction in symptoms in the model system will indicate an effective antibody. Optimizing the dose for the model system is within the knowledge of one of ordinary skill in the art. Thus, the above models can be used to provide additional guidance as to the desired doses.

### Example 11

### A Method of Reducing the Symptoms of Delayed-Type Hypersensitivity, Contact Hypersensitivity, and Arthritis in a Patient

A patient suffering from delayed-type hypersensitivity, contact hypersensitivity, and/or arthritis, is identified. A dosage of, for example, 5 mg/kg of a VLA-1 antibody is administered by intravenous injection to the patient. The dosage can be adjusted so that it is enough to be effective in preventing delayed-type hypersensitivity, contact hypersensitivity, and/or arthritis (the amount can be identified by the methods described in Example 10) A booster administration is given three weeks later, and every three weeks thereafter. The VLA-1 antibody causes a partial or complete suppression of the symptoms of delayed-type hypersensitivity, contact hypersensitivity, and/or arthritis in the patient.

### Example 12

### General Epitope Mapping

The epitope for each of the antibodies can also be determined. One can do this in a variety of ways. For example, one can create variants of the alpha-1 subunit of VLA-1 and apply the antibody to the variant to see if the antibody can still bind to VLA-1 or the alpha-1 subunit of VLA-1 with the same or similar affinity. Any mutation that alters the affinity of the antibody for the alpha 1 subunit can be part of the epitope. By altering multiple positions via multiple variants, one can determine all of the amino acids that are involved in the formation of the epitope. By mapping the positions onto the structure of the alpha-i subunit, one can see if the residues cluster together in three dimensional space, suggesting that the residues play a direct role in forming the epitope, or if those residues are distributed throughout the surface of the protein, suggesting that those residues may be having additional or alternative effects on the alpha-1 subunit. Of course, functionality tests of the variant alpha-1 subunits can also reveal this. One way of creating the variants is to make chimeric alpha-1 subunits, for example between human and mouse proteins, thus, altering large portions or segments of the protein without necessarily impacting the proteins overall shape or function.

### Example 13

### Diagnosis of a VLA-1 Inflammation Disorder

A patient to be diagnosed for a possible VLA-1 related disorder is identified. A sample from the patient is obtained from the tissue that may be involved in the inflammation disorder. A sufficient amount of a VLA-1 antibody is contacted to the sample. Excess VLA-1 antibody is rinsed away and the amount of VLA-1 antibody remaining in the sample is determined. This amount is compared to an amount of the VLA-1 antibody that remains in a sample that is experiencing an inflammation disorder. The similarities in the amount of VLA-1 antibody remaining and the sample in which it occurs can be used to further determine which inflammation disorder is involved. Additionally, the amount of VLA-1 antibody from the patient can also be compared to the amount VLA-1 antibody remaining in a healthy sample.

### Example 14

### A Method of Reducing the Symptoms of Asthma in a Patient

A patient suffering from asthma is identified. A dosage of 5 mg/kg of a VLA-1 antibody (e.g., 1.7 and/or 2.7 or fragments thereof) that is effective in preventing asthma (which can be identified by the methods described in Example 10) is administered by intravenous injection to the patient. A booster administration is given three weeks later, and every three weeks thereafter. The VLA-1 antibody causes a partial or complete suppression of the symptoms of asthma in the patient.

### Example 15

### A Method of Reducing the Symptoms of Inflammatory Bowel Disease in a Patient

A patient suffering from inflammatory bowel disease is identified. A dosage of, for example, 5 mg/kg of a VLA-1 antibody (e.g., 1.7 and/or 2.7) is administered by intravenous injection to the patient. The dosage can be adjusted so that it is enough to be effective in preventing inflammatory bowel disease (the amount can be identified by the methods described in Example 10). A booster administration is given three weeks later, and every three weeks thereafter. The VLA-1 antibody causes a partial or complete suppression of the symptoms of inflammatory bowel disease in the patient.

### Example 16

### A Method of Reducing the Symptoms of a VLA-1 Mediated Disorder in a Patient

A patient suffering from a VLA-1 mediated disorder is identified. This can be achieved in a variety of ways, including identifying a patient that has one of the disorders disclosed herein, identifying a patient with similar symptoms, or identifying a patient or test subject that is responsive to the presently disclosed antibodies. A dosage of, for example, 5 mg/kg of a VLA-1 antibody is administered by intravenous injection to the patient so that it is effective in preventing inflammatory bowel disease (the amount can be identified by the methods described in Example 10). A booster administration is given three weeks later, and every three weeks thereafter. The VLA-1 antibody causes a partial or complete suppression of the symptoms of inflammatory bowel disease in the patient.

### Incorporation by Reference

All references cited herein, including patents, patent applications, papers, text books, and the like, and the references cited therein, to the extent that they are not already, are hereby incorporated herein by reference in their entirety.

### Equivalents

The foregoing written specification is considered to be sufficient to enable one skilled in the art to practice the invention. The foregoing description and Examples detail certain preferred embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

What is claimed is:

1. A human antibody or antigen-binding portion thereof that binds to very late antigen-1 (“VLA-1”), wherein the isolated fully human antibody binds to the alpha subunit of very late antigen-1 with a K_{D} of no more than about 200 pM, and wherein said antibody inhibits the interaction between very late antigen-1 and a molecule selected from the group consisting of collagen IV, collagen I, and laminin.

2. The human antibody, or antigen-binding portion thereof of claim 1, wherein the human antibody binds to very late antigen-1 with a K_{D} of no more than about 100 pM.

3. The human antibody, or antigen-binding portion thereof of claim 1, wherein the human antibody, or antigen-binding portion thereof, binds to very late antigen-1 with a K_{D} of no more than about 50 pM.

4. The human antibody or antigen-binding portion thereof of claim 1, wherein the human antibody, or antigen-binding portion thereof, binds to very late antigen-1 with a K_{D} of no more than about 25 pM.

5. The human antibody or antigen-binding portion thereof of claim 1, wherein the K_{D} is defined in the presence of at least 1 mM of a diavalent cation.

6. The human antibody or antigen-binding portion thereof of claim 1, wherein said antibody, or antigen-binding portion thereof, does not bind to mouse very late antigen-1.

7. The human antibody or antigen-binding portion thereof of claim 1, wherein said antibody, or antigen-binding portion thereof, does not bind to very late antigen-2.

8. The human antibody or antigen-binding portion thereof of claim 1, wherein said antibody, or antigen-binding portion thereof, inhibits the binding of recombinant CD49a I domain of very late antigen-1 (“VLA-1”) to collagen.

9. The human antibody or antigen-binding portion thereof of claim 1, wherein said antibody, or antigen-binding portion thereof, inhibits the collagen IV-mediated transmigration of activated T cells.

10. The human antibody or antigen-binding portion thereof of claim 1, wherein said antibody, or antigen-binding portion thereof, inhibits the effector phase of delayed type of hypersensitivity in vivo.

11. The human antibody or antigen-binding portion thereof of claim 1, wherein the antibody, or antigen-binding portion thereof, binds to amino acid position E66 on CD49a.

12. The human antibody or antigen-binding portion thereof of claim 1, wherein the antibody, or antigen-binding portion thereof, binds to amino acid position K142, S162, or both on CD49a.

13. The antigen-binding portion of claim 1, wherein the antigen-binding portion is a Fab.

14. A human antibody or antigen-binding portion thereof that binds to an alpha subunit of very late antigen-1 (“VLA-1”), wherein the human antibody, or antigen-binding portion thereof, has an IC₅₀ of no more than about 5 nM for preventing VLA-1 mediated adhesion to collagen IV.

15. The human antibody or antigen-binding portion thereof of claim 14, wherein the antibody, or antigen-binding portion thereof, has an IC₅₀ of no more than about 500 pM.

16. The human antibody or antigen-binding portion thereof of claim 14, wherein the antibody, or antigen-binding portion thereof, has an IC₅₀ of no more than about 10 pM.

17. A human antibody or antigen-binding portion thereof that binds to an alpha subunit of very late antigen-1 (“VLA-1”), wherein the human antibody, or antigen-binding portion thereof, has an IC₅₀ of no more than 5 nM for inhibiting an interaction between immobilized collagen IV and an I domain of very late antigen-1.

18. A method of treating a subject suffering from a very late antigen-l (“VLA-1”) mediated disorder, the method comprising:
selecting a subject in need of treatment for a very late antigen-1 mediated disorder; and
administering to said subject a therapeutically effective dose of a human monoclonal antibody or antigen-binding portion thereof that specifically binds to very late antigen-1, wherein said antibody has a K_{D} of no more than 200 pM, and wherein said antibody inhibits the interaction between very late antigen-1 and collagen IV.

19. The method of claim 18, wherein the very late antigen-1 mediated disorder is an inflammatory disorder.

20. The method of claim 19, wherein the inflammatory disorder is inflammatory bowel disease.

21. A kit for treating very late antigen-1 related disorders, said kit comprising a human very late antigen-1 antibody, or antigen-binding portion thereof, of claim 1 in a unit dose; and instructions for administering the unit dose of the very late antigen-1 antibody to a subject.

22. A kit for detecting very late antigen-1 in a cell or to screen for very late antigen-1 related disorders, the kit comprising:
a human antibody, or antigen-binding portion thereof, that binds to very late antigen-1 with a K_{D} of no more than about 200 pM; and
a means for indicating a binding of the antibody with very late antigen-1.

23. An isolated nucleic acid sequence encoding the antibody, or antigen-binding portion thereof, of claim 1.

24. A host cell transformed with the nucleic acid sequence of claim 23.

25. A method of diagnosing a very late antigen-1 (“VLA-1”) mediated disorder comprising using the human very late antigen-1 antibody, or antigen-binding portion thereof, of claim 1 to detect the level of very late antigen-1 in a patient sample, wherein an amount of very late antigen-1 in the sample that is higher than a predetermined amount indicates that the patient is suffering from a very late antigen-1 mediated disorder.

26. A human antibody or antigen-binding portion thereof that binds to very late antigen-1 (“VLA-1”), wherein the human antibody or antigen-binding portion thereof binds to an epitope comprising amino acid position E66 on CD49a.

27. A human antibody or antigen-binding portion thereof that binds to very late antigen-1 (“VLA-1”), wherein the human antibody or antigen-binding portion thereof binds to an epitope comprising amino acid position K142 on CD49a.

28. The human antibody or antigen-binding portion thereof of claim 27 wherein the epitope further comprises amino acid position S162 on CD49a.

29. A human antibody or antigen-binding portion thereof that binds to very late antigen-1 (“VLA-1”), wherein the human antibody or antigen-binding portion thereof binds to an epitope comprising amino acid position S 162 on CD49a.

30. A composition comprising the human antibody or antigen-binding portion thereof of claim 1 and a pharmaceutically acceptable carrier.
